(19) United States
(12) Patent Application Publication
(10) Pub. No.: US 2026/0241075 A1
(43) Pub. Date: Aug. 20, 2026

(54) RADIATION SPACER HYDROGELS, METHODS OF FORMING, AND METHODS OF USE

(71) Applicant: CLEARSTREAM TECHNOLOGIES LIMITED, Enniscorthy, County Wexford (IE)

(72) Inventors: Jordan ADDISON, Gilbert, AZ (US); Dylan BEYHL, Phoenix, AZ (US); Nicholas MOWREY, Scottsdale, AZ (US); Heather A. STORM, Phoenix, AZ (US); Luke WINTERSTEIN, Tempe, AZ (US)

(73) Assignee: CLEARSTREAM TECHNOLOGIES LIMITED, Enniscorthy, County Wexford (IE)

(21) Appl. No.: 19/162,115

(22) PCT Filed: Mar. 10, 2023
(86) PCT No.: PCT/US2023/064171

§ 371 Date: Sep. 4, 2025

(51) Int. Cl. A61L 27/52; A61L 27/02; A61L 27/18; A61L 27/22; A61L 27/54

(52) U.S. Cl. CPC A61L 27/52; A61L 27/025; A61L 27/18; A61L 27/227; A61L 27/54; A61L 2300/416; A61L 2300/44

(57) ABSTRACT

The present disclosure concerns hydrogel compositions designed to occupy space within a cavity or space within a subject, as well as various additives or formulations thereof that allow the hydrogels to provide a therapeutic or physiological benefit to the subject in addition to the structural support provided.

### TECHNICAL FIELD

The present specification relates generally to hydrogels and methods of making and using said hydrogels, and more specifically to hydrogels used in the separation and treatment of tissues.

### BACKGROUND

Patients receiving radiation therapy are susceptible to complications arising from scatter radiation that delivers harmful doses of radiation to nearby organs. Radiation toxicity to nearby adjacent organs can cause health problems. Hydrogel spacers that are used for implantation to create space between a targeted tissue and a non-targeted tissue to minimize collateral radiation may be difficult to deliver. For example, as the hydrogel is delivered it can take too long to polymerize, allowing migration of the gel from the intended delivery location. Such a circumstance leads to a non-targeted tissue receiving higher doses of collateral radiation.

Accordingly, there exists a need to reduce the occurrence and severity of these devastating side effects for patients undergoing radiation therapy by reducing the dose of radiation delivered to adjacent organs at risk.

### SUMMARY

The present disclosure concerns hydrogel compositions. In some aspects, the hydrogels are described with respect to application to filling or occupying a space or cavity within a subject, such as a human subject. It will be appreciated that the hydrogels as described herein are not so limited in use. The hydrogels as described herein can be varied, such that they can be formulated to degrade slowly or relatively rapidly. The hydrogels similarly may be varied in their rheology or elastic modulus.

In aspects, the present disclosure concerns a hydrogel composition for in situ formation within a cavity that includes a polymerized complex of at least one monomeric unit, at least one complexing molecule and an aqueous solution. The monomeric unit may include a terminal structure, a linker and a core and wherein the complexing molecule is chosen from recombinant albumin, polyethyleneimine (PEI), and poly-lysine. The linker may be of polyethylene glycol (PEG). The linker may be of sufficient length to provide the hydrogel with a molecular weight (MW) of between 1 kDa and 100 kDa.

In aspects, the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In aspects, the core is chosen from pentaerythritol, hexaglycerol, tripentaerythritol, or glycerol. The monomeric unit may accordingly have 2, 4, or 8 linker arms extending from the core.

In aspects, the hydrogel composition is formed, at least in part, through an ester of the monomeric unit complexing with an amine or imine of the complexing molecule.

In aspects, the hydrogel composition may possess a molar ratio of ester of the monomeric unit to amine or imine of the complexing molecule is of from 0.05 to 3. In some aspects, the molar ratio is of 1.5 to 2.

In aspects, the complexing molecule comprises from 2 to 60% weight/volume (w/v) of the hydrogel composition. In aspects, the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In aspects, the hydrogel composition has a pH of between 7.5 and 11.0.

In aspects, the monomeric unit is NHS-PEG-SG-8, NHS-PEG-SG-4, or NHS-PEG-SG-2. In aspects, the monomeric unit is NHS-PEG-SS-8, NHS-PEG-SS-4, or NHS-PEG-SS-2. In aspects, the monomeric unit is NHS-PEG-SCM-8, NHS-PEG-SCM-4, or NHS-PEG-SCM-2.

In aspects, the complexing molecule is rHA. In aspects, the complexing molecule is PEI.

In aspects, the hydrogel may include an embedded component, such as a therapeutic agent, a radiosensitizing agent, a radioprotective agent, gas nanobubbles, a peroxide, a compound that allows for the production of reactive oxygen species from radiotherapy, or a microparticle.

In some aspects, the microparticle is of a biodegradable polymer. In some aspects, the microparticle includes a therapeutic agent and/or a tissue marker and/or a radioactive isotope or a compound including at least one radioactive element. In some aspects, the microparticle includes a radiopaque marker, such as methylated TIBA (TIBA-Me).

In some aspects, the hydrogel composition includes at least one osmotic component. In some aspects, the hydrogel includes two or more layers. In some aspects, the layers have different osmotic concentrations. In some aspects, the hydrogel has a different osmotic concentration with respect to a subject's tissue or the cavity the hydrogel will occupy.

In aspects, the present disclosure concerns a method to prepare a hydrogel composition by preparing a first solution of a monomeric unit reconstituted in a first aqueous solution; preparing a second solution of a complexing molecule reconstituted in a second aqueous solution; and contacting the first solution with the second solution as both are poured into a cavity space in a subject to form a polymerized complex therein. In some aspects, the cavity space is between the subject's rectum and prostate. In some aspects, the cavity space is located in the subject's breast tissue.

In some aspects, the present disclosure concerns the use of the hydrogel composition as described herein to separate rectal tissue from prostate tissue in a subject.

In aspects, the present disclosure also includes kits to prepare the hydrogel compositions as described herein.

### DETAILED DESCRIPTION

Embodiments described herein are generally directed to hydrogels, methods of forming, and methods of using the same. The hydrogels generally include a polymerized complex of at least one monomeric unit, at least one complexing molecule and an aqueous solution, wherein the monomeric unit comprises a terminal structure, a linker and a core and wherein the complexing molecule is chosen from recombinant albumin, polyethyleneimine (PEI), and poly-lysine. These and other features and embodiments of the bioreactors are disclosed in greater detail herein.

The present disclosure, in one form, is related to radiation spacer hydrogels that incorporate an implantable hydrogel spacer for the separation of tissues to protect against collateral radiation as well as systems and methods that incorporate the same.

Further, the radiation spacer hydrogels described herein may be implanted for a length of a subject's treatment, but additional procedures increase the risk of complications. As such, the hydrogel, systems, and methods described herein include biodegradable components that can be adapted to a subject's specific needs. As used herein “adapted” means that the radiation spacer hydrogels is particularly formulated, shaped, and sized to meet the subject's specific anatomy and treatment goals. “Adapted” also means that the materials forming the hydrogel are selected for or tuned to a specific degradation profile that aligns with the subject's anticipated treatment length and/or needs.

The radiation spacer hydrogel disclosed herein is designed such that the hydrogel is capable of creating separation between a targeted tissue and a non-targeted tissue. As used herein, the term “separation” or “displacement” refers to filling a void between the targeted tissue and the non-targeted tissue or moving the targeted tissue and the non-targeted tissue such that the radiation spacer hydrogels creates and fills a void between the tissues. This space created by the radiation spacer hydrogel protects the non-targeted tissue from exposure or unintended side effects during treatment. The radiation spacer hydrogels can reduce harmful effects of radiation therapy of the non-targeted tissue, allow for improved targeting of the targeted tissue, allow for higher doses of radiation, provide additional and/or alternative treatment and/or allow for shorter treatment times.

Radiation therapy is an excellent treatment option for treatment of various cancers. However, radiation exposure can cause unintended side effects in adjacent organs. A radiation spacer hydrogel can be implanted to avoid collateral radiation and minimize injury to nearby organs by providing a space between the target organ or tissue and nearby organs or tissues at risk.

As used herein, the term “targeted tissue” refers to a tissue or organ in need of radiation therapy or other treatment. As used herein, the term “non-targeted tissue” refers to a tissue or organ adjacent to the targeted tissue, where the non-targeted tissue is at risk of side effects from the treatment of the targeted tissue. In some aspects, the non-targeted tissue is at risk of collateral radiation.

It is also an aspect of the present disclosure that the hydrogel spacers disclosed herein may also be used in other medical procedures and treatment, including, but not limited to, vessel occlusion, punctal occlusion, ductal occlusion, and other procedures and treatments that require obstructing a lumen in a subject. Additionally, the hydrogel spacer may be used in medical procedures that require creating space in a subject, including but not limited to, orbital volume augmentation, dental procedures, tissue expansion for reconstructive surgery, vocal fold procedures, and the like.

An advantage of the present disclosure is that the biodegradable components reduce the need for follow up procedures. Additionally, the quick gelling time of the hydrogels prevents movement of the hydrogel spacer into unintended areas. Additionally, the present disclosure provides hydrogel spacers that can be tailored to a subject's unique anatomy and treatment needs.

### Hydrogel

The present disclosure concerns, at least in part, the presence of a hydrogel, either alone or as a body encapsulating other materials and/or agents. A hydrogel refers to a polymerized network of water-insoluble monomer units cross-linked with a complexing molecule that retain water therein. A hydrogel may be of one single type of cross-linked monomer units or may be a combination of two or more types of monomer units. A hydrogel may be a cross-linked network of at least one type of monomeric units, including two, three, four, five, six, seven, eight, and more different monomer units. It will be appreciated that a unit may refer to an assembled molecule of at least two parts, each part providing different functionalities to the monomeric unit.

The hydrogels include cross-linking between monomeric units and a complexing molecule within the hydrogel. Cross-linking refers to a bond or attraction point between different moieties and/or different monomers. In aspects, the bond is a chemical bond, such as a covalent bond, an ionic bond, or a metallic bond. In aspects, the bond or attraction may include van der Waal forces, hydrogen bonding, Keesome forces, Coulombic interactions, Pauli repulsions, halogen bonds, and combinations thereof. It will be appreciated that a hydrogel need not contain just one type of bond between moieties, but can instead feature two or more types of bond or attraction throughout the formed hydrogel. In some aspects, the hydrogel may be a chemical hydrogel wherein covalent bonds link strands together. In some aspects, the hydrogels may be a physical hydrogel wherein hydrogen bonds, entanglements, hydrophobic interactions, and similar physical interactions form the gel. It will be appreciated that a hydrogel may also contain a combination of chemical and physical interactions.

In some aspects, the hydrogels include monomeric units cross-linked with the complexing molecule by bonds or attractions between reactive atoms, sub-molecules, or moieties within each monomeric unit and complexing molecule. In some aspects, a monomeric unit may cross-link with another atom or moiety in the same monomeric unit or “self” cross-link. In some aspects, a moiety or atom within one monomeric unit will bond or attract with another moiety or atom in a different monomeric unit, either of the same type of molecule or of a different molecule.

In some aspects, the bond or attraction that leads to cross-linking may be between an oxygen, a primary/secondary/tertiary amine, a peroxide, a superoxide, a nitrogen, a sulfur, a phosphorous, a boron, a lithium, a heme, an iron, an aluminum, a silicon, a metal ion, a carboxyl, a thiol, a hydroxyl, a carbon, a carbonyl, a carbonate, a carboxylate, a carboalkoxy, a ketone, a imide, a halogen, an acyl halide, a hydroperoxy, an ether, a hemmiacetyl, an acetal, an orthoester, a methylenedioxy, a carboxamide, an amidine, a primary/secondary ketimine, a primary/secondary aldimine, an azide, an azo, a cyanate/isocyanate, a nitrate, a nitrite, a nitrile, a nitrsooxy, a nitro, a nitroso, an oxime, a pyridyl, a carbamate, a sulfhydryl, a sulfide, a disulfide, a sulfinyl, a sulfonyl, a sulfino, a sulfo, a thiocyanate, a carbonothioyl, a carbothioic acid, a thiolester, a thionoester, a carbodithioic acid, a carbodithio, a phosphino, a phosphono, a phosphate, a phosphodiester, a borono, a boronate, a borino, a borinate, an alkykllithium, an alkylmagnesium halide, an alkylaluminum, a silyl ether, or similar.

In some aspects, the monomeric units of the hydrogel include a structure of a linker or a spacer with available moieties along the length thereof for binding other monomers or water, such as polyethylene glycol or repeats thereof, attached to a core or central moiety at one end and a terminal structure at the opposing end. In some aspects, the terminal structure is an active ester. In some aspects, the central moiety is a polyol or hydroxyl core. In aspects, one or more linkers may bind the central moiety or core and extend outward therefrom. In aspects, 2, 4, or 8 linkers may be attached to the core or central moiety. Each linker connected to the core or central moiety may accordingly be considered as a figurative appendage or arm extending therefrom.

In aspects, the terminal structure includes a cyclical or ringed organic compound conjugated with a primary amine through a stable amide bond to the carboxyl group of a carboxylic acid, an alkanedioic acid, an alkenedioic acid, or a branched dioic acid. In some aspects, the primary amine is linked to an alkanedioic acid, such as a linear dicarboxylic acid. In some aspects, the terminal structure is a succinimidyl ester. In aspects, the succinimide is N-hydroxysuccinimide (NHS). In other aspects, the succinimide is a further substituted NHS. In aspects, the terminal structure includes a cyclical or ringed organic compound conjugated to the linker through an appended carboxyl group of a carboxylic acid, such as benzoic acid (BA) or a methylated form thereof. In aspects, the terminal structure may include BA, NHS, acrylamide, biotin, COOH, an alkyne, a halogen (such as chloride), an epoxide, hydrazide, norborene, hydroxyl, azide, amine, acrylate, dibenzocyclooctyne (DBCO), glutamic acid, glutaramide acid, succinimidyl glutaramide ester (GAS), maleimide, para-nitrophenyl carbonate (NPC), orthopyridyl disulfide (OPSS), acetic acid, carboxyl methyl, glutaric acid, succinic acid, glutaramide acid, succinamide acid, succinimidyl succinamide ester (SAS), thiol (or SH), tosylate, vinylsulfone, or combinations thereof.

In aspects, the linear dicarboxylic acid is saturated. In some aspects, the dicarboxylic acid is unsaturated. In aspects, the alkanedioic/alkenedioic/branched acid is selected from oxalic acid, malonic acid, succinic acid, itaconic acid, glutaric acid, adipic acid, pimelic acid, suberic acid, azelaic acid, tartaric acid, malic acid, and/or citraconic acid. In some aspects, the primary amine is linked to glycolic acid. In some aspects, the terminal structure is an ester of NHS and one of glycolic acid, succinic acid, and glutaric acid.

In some aspects, the terminal structure is a succinimidyl carboxymethyl ester (SCM). In some aspects, the terminal structure is a succinimidyl succinate ester (SS). In some aspects, the terminal structure is a succinimidyl glutarate (SG). In some aspects, the terminal structure includes at least one of NHS-SG, NHS-SS, and NHS-SCM.

In aspects, the terminal structure may include an acrylate linked to the linker as a methyl ether.

In some aspects, the central moiety includes a hydroxyl or polyol core. In some aspects, the polyol core includes pentaerythritol, hexaglycerol, tripentaerythritol, or glycerol. In aspects, the central moiety is a glycerol-based core that allows for the presence of “arms” to extend therefrom. A linker may provide a connection between the terminal structure and the core. For example, a glycerol provides for two arms, pentaerythritol can provide up to four arms, hexaglycerol can provide up to eight arms, and tripentathritol can provide up to eight arms. It can be a further aspect of the linker and/or terminal structure that provides reactive points for cross-linking, such as exposed oxygens, sulfurs, hydroxyl groups, amines, and the like. In some aspects, the linker may include one or more ethylene glycol molecules.

In aspects, the linker is an ethylene glycol or a polyethylene glycol (PEG) or a linear or branched chain thereof. In aspects, a PEG-chain can link the terminal structure (TS) to the core. Formula I sets forth the basic structure:

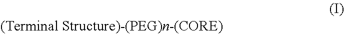

It will be appreciated that the number for n can vary depending on the desired final molecular mass. In aspects, n is an integer from 1 to 10, 1 to 100, 1 to 500, or so on, including 2, 3, 4, 5, 6, 7, 8, 9, 10, 11, 12, 13, 14, 15, 16, 17, 18, 19, and 20. In aspects, n is greater than or equal to 20, greater than or equal to 25, greater than or equal to 30, greater than or equal to 35, greater than or equal to 40, greater than or equal to 45, greater than or equal to 50, greater than or equal to 55, greater than or equal to 60, greater than or equal to 65, greater than or equal to 70, greater than or equal to 75, greater than or equal to 80, greater than or equal to 85, greater than or equal to 90, greater than or equal to 95, or greater than or equal to 100.

The hydrogels of the present disclosure include a monomeric unit of the terminal structure-linker-core. In some aspects, the core is linked to two or more linkers, providing a multiple armed monomer. In some aspects, the core may have 2 arms or otherwise be a linear monomer of:

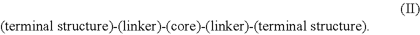

It will be appreciated that for such a two armed structure, a single ethylene glycol molecule may function as the core. In some aspects, the core may have 4 arms and have a structure of:

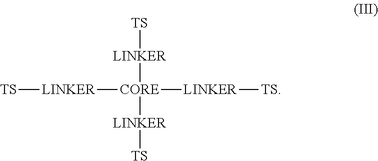

In aspects, the core may have eight arms with a structure of:

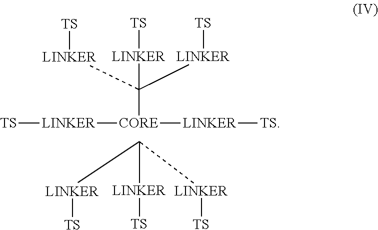

In aspects, the core allows for three, four, five, six, seven, or eight arms. In some aspects, the number of arms can be further increased through additional modifications to the core. For example, PEG molecules can be pre-reacted to provide branched structures that each provide an available oxygen for each arm to attach.

In aspects, the hydrogel may include a monomeric unit of a terminal structure of a succinimide, a PEG linker, and a glycerol or glycerol derived core. The succinimide can be an NHS. The linker can be a PEG sufficient to provide a molecular weight (MW) of from about 1,000 Daltons (Da) to about 100,000 Da. The core can be pentaerythritol, hexaglycerol, tripentaerythritol, or glycerol. The monomer can be of 2, 4, or 8 arms.

In aspects, the hydrogel may include a monomeric unit of a terminal structure of a succinimidyl glutarate, a PEG linker, and a glycerol or glycerol derived core. The succinimidyl glutarate can be an NHS-SG. The linker can be a PEG sufficient to provide a molecular weight (MW) of from about 1,000 Daltons (Da) to about 100,000 Da. The core can be pentaerythritol, hexaglycerol, tripentaerythritol, or glycerol. The monomer can be of 2, 4, or 8 arms or NHS-PEG-SG-2, NHS-PEG-SG-4, and NHS-PEG-SG-8, respectively.

In aspects, the hydrogel may include a monomeric unit of a terminal structure of a succinimidyl carboxymethyl, a PEG linker, and a glycerol or glycerol derived core. The succinimidyl carboxymethyl can be an NHS-SCM. The linker can be a PEG sufficient to provide a molecular weight (MW) of from about 1,000 Daltons (Da) to about 100,000 Da. The core can be pentaerythritol, hexaglycerol, tripentaerythritol, or glycerol. The monomer can be of 2, 4, or 8 arms or NHS-PEG-SCM-2, NHS-PEG-SCM-4, and NHS-PEG-SCM-8, respectively.

In aspects, the hydrogel may include a monomeric unit of a terminal structure of a succinimidyl succinate, a PEG linker, and a glycerol or glycerol derived core. The succinimidyl succinate can be an NHS-SS. The linker can be a PEG sufficient to provide a molecular weight (MW) of from about 1,000 Daltons (Da) to about 100,000 Da. The core can be pentaerythritol, hexaglycerol, tripentaerythritol, or glycerol. The monomer can be of 2, 4, or 8 arms or NHS-PEG-SS-2, NHS-PEG-SS-4, and NHS-PEG-SS-8, respectively.

In aspects, the terminal structure may be substituted with one or more halogens, such as iodine, to render the monomeric unit radiopaque. In some aspects, the terminal structure may be a benzoic acid (BA) or a substituted variant thereof. In some aspects, the benzene ring of BA may be substituted with one or more halogens. In some aspects, the halogen may be iodine. In some aspects, the terminal structure may be a mono-, di-, or tri-iodo BA. In some aspects, the terminal structure is tri iodobenzoic acid (TIBA). In some aspects, the monomer is a TIBA-SG, TIBA-SS, or TIBA-SCM. In some aspects, the linker can be a PEG sufficient to provide a molecular weight (MW) of from about 1,000 Daltons (Da) to about 100,000 Da. The core can be pentaerythritol, hexaglycerol, tripentaerythritol, or glycerol. The monomer can be of 2, 4, or 8 arms, such as TIBA-PEG-2, TIBA-PEG-4, TIBA-PEG-8, TIBA-PEG-SS-2, TIBA-PEG SG-2, TIBA-PEG-SCM-2, TIBA-PEG-SS-4, TIBA-PEG-SG-4, TIBA-PEG-SCM-4, TIBA-PEG-SCM-8, TIBA-PEG-SG-8, and TIBA-PEG-SCM-8.

In aspects, the hydrogels further include complexing molecules or macromolecules that polymerize with the monomeric units. In some aspects, a hydrogel may include complexing molecules such as peptides, polypeptides, or proteins. In aspects, the hydrogel is a polymerized network of cross-linked monomer(s) and peptides/polypeptides/proteins. It will be appreciated that peptide refers to two or more amino acids linked via a peptide bond, with a polypeptide referring to a series of chain of multiple linked amino acids and a protein referring to a full-length expressed gene or chimera thereof.

In aspects, the complexing molecules or macromolecules include a central supporting backbone with one or more reactive side chains appended thereto. For example, amino acids include a central peptide bond between the amino and carboxylic acid groups, but also possess side chains. Depending on the amino acid, the side chain can be reactive, such as with arginine, histidine, lysine, aspartate, glutamate, serine, threonine, asparagine, glutamine, cysteine, methionine, and tyrosine provide reactive moieties. Similarly, molecules such as polyethylenimine (PEI) provide reactive amine groups within the complexing molecules that are available for reacting with the monomeric units. Similarly, polylysine, sulfone, polyphthalamide (PPA), polyphenylene (PPS), polyether ether ketone (PEEK), or combinations thereof can be provided as the complexing molecule.

In aspects, the hydrogel may form through interactions between the primary amide-ester bond between the terminal group and the linker and a primary amine in the complexing molecule, such as an amine within PEI or a primary amine of an amino acid side chain in a protein.

In aspects, the complexing molecule is a recombinant protein and/or synthetic protein. In some aspects, the protein can be a commonly found protein in most animal species. Such may provide for minimal reaction by the immune system. In aspects, the protein is albumin. In some aspects, the protein is human albumin, including recombinant human serum albumin (rHSA or rHA) and/or synthetic human serum albumin, such as that formed by solid phase peptide synthesis and solution phase synthesis. In some aspects, the complexing molecule may be poly-lysine or a polypeptide with multiple lysine residues.

In some aspects, the complexing molecule has a molecular weight of from about 1,000 Da to about 100,000 Da. For example, rHSA has a MW of about 66 kDa. PEI has a variable MW depending on the length of the monomer. Similarly, a poly-lysine polypeptide MW can be constructed based on a desired size.

In some aspects, the complexing molecule is provided within the hydrogel at about 2 to about 60% weight/volume (w/v), including about 3, 4, 5, 6, 7, 8, 9, 10, 15, 20, 25, 30, 35, 40, 45, 50, and 55% w/v. In some aspects, the complexing molecule is of from about 5 to about 50% w/v, from about 5 to about 40% w/v, from about 5 to about 30% w/v, from about 5 to about 20% w/v, from about 5 to about 10% w/v, from about 10 to about 50% w/v, from about 10 to about 40% w/v, from about 10 to about 30% w/v, from about 10 to about 20% w/v, from about 20 to about 50% w/v, from about 20 to about 40% w/v, from about 20 to about 30% w/v, from about 30 to about 50% w/v, from about 30 to about 40% w/v, or from about 40 to about 50% w/v. In some aspects, the complexing molecule is albumin (including human albumin) and/or PEI and/or poly-lysine.

In some aspects, the monomeric unit:complexing molecule mass ratio is provided at about 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7. In some aspects, the monomeric unit is of 2, 4, or 8 arms with PEG as the linker. In some aspects, the monomeric unit is of from about 2 kDa to about 40 kDa in MW. In some aspects, the complexing molecule is PEI and/or rHA.

In aspects of the present disclosure, the hydrogel forms, at least in part, through reactions between the oxygen linking the linker to the terminal group and primary amines in the complexing molecule. For example, NHS-PEG-SG-8 will react with the imines in PEI or primary amines available in the side chains of the amino acids of rHA, such as lysine, arginine, asparagine, and glutamine. In some aspects, the ester (also referred to as NHS-ester or NHS) to amine molar ratio determines certain qualities of the hydrogel. In some aspects, the ester:amine (or NHS:amine) ratio is between 0.05 and 3. In some aspects the ester/NHS:amine molar ratio is over 1.00. In some aspects the ester/NHS:amine molar ratio is of about 1.5 to about 2 or about 2.75.

In some aspects, the hydrogel includes a combination of PEG-SG-8 (or NHS-PEG-SG-8 or TIBA-SG-8) and rHA or PEI, of PEG-SG-4 (or NHS-PEG-SG-4 or TIBA-SG-4) and rHA or PEI, of PEG-SG-2 (or NHS-PEG-SG-2 or TIBA-SG-2) and rHA or PEI, of PEG-SS-8 (or NHS-PEG-SS-8 or TIBA-SS-8) and rHA or PEI, PEG-SS-4 (or NHS-PEG-SS-4 or TIBA-SS-4) and rHA or PEI, of PEG-SS-2 (or NHS-PEG-SS-2 or TIBA-SS-2) and rHA or PEI, PEG-SCM-8 (or NHS-PEG-SCM-8 or TIBA-SCM-8) and rHA or PEI, of PEG-SCM-4 (or NHS-PEG-SCM-4 or TIBA-SCM-4) and rHA or PEI, or of PEG-SCM-2 (or NHS-PEG-SCM-2 or TIBA-SCM-2) and rHA or PEI.

In some aspects, the hydrogel is of a monomeric unit of PEG-SG, -SS, or SCM at a MW of from about 10 kDa to about 20 kDa. In some aspects, the monomeric unit is added to a solution of from about 10 to about 20 wt. %/volume complexing unit, such as rHA or PEI or polylysine. In some aspects, increasing the MW of PEG in the linker can allow for the hydrogel to retain water.

In aspects of the present disclosure, the hydrogels are prepared with one or more aqueous solutions, such that water is retained or trapped within the formed hydrogel. In aspects, the aqueous solution include or is entirely of water. Such water maybe be of sufficient quality or purity so as to minimize introducing unwanted contaminants when placed within a space or cavity within a living organism so as to reduce risk of generating or exaggerating an immune response. Such may include filtered water, deionized water, distilled water, reverse osmosis water, or similar and combinations thereof. In aspects, the aqueous solution may include ions, such as cations, anions, or combinations thereof. In aspects, the aqueous solution may include a salt, or the ions thereof, such as sodium, magnesium, manganese, potassium, chloride, boride, iodide, sulfate, sulfite, nitrate, nitrite, carbonate, bicarbonate, ammonia, phosphate, calcium, or combinations thereof. In aspects, the aqueous solution may contain one or more buffers. In aspects, the aqueous solution may include one or more biochemical molecules such as NADPH (nicotinamide adenine dinucleotide phosphate), NAD (nicotinamide adenine dinucleotide), ATP (adenine triphosphate), CTP (cytosine triphosphate), GTP (guanosine triphosphate), TTP (thymidine triphosphate), Acetyl COA, amino acids, sugars, saccharides, polysaccharides, purines, pyrimidines, cytokines, growth factors, or similar. It will be apparent that the aqueous solution may include any soluble material therein, such as a hydrophilic therapeutic agent, contrast agent, surfactant, or similar. Such may include iohexol, sodium bicarbonate, sodium stearate, sodium docusate, an alkyl ether phosphate, benzalkonium chloride, perfluorooctanesulfonate, carboxy methyl cellulose (CMC), EO/PO (ethylene oxide and propylene oxide) block copolymer (e.g. PLURONIC), PEG fatty esters, PEG omega-3 fatty esters and alcohols, glycerol fatty esters, sorbitan fatty esters, PEG glyceryl fatty esters, PEG sorbitan fatty esters, sugar fatty esters, PEG sugar esters, polysorbate 20, polysorbate 40, polysorbate 60, p-isononylphenoxypolyglycidol, PEG laurate, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG lauryl ether, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside, sodium docusate, sorbitol, urea, BHT, BHA, PEG-sorbitan monolaureate, petrolatum, methyl stearate or a combination thereof.

In aspects, the aqueous solutions may be prepared or adjusted to a desired pH. In some aspects, an aqueous solution may have an alkaline pH of between about 7.5 and 11.0, including 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, and 10.9. In some aspects, an aqueous solution may have an acidic pH of between about 3.5 and 6.5, including 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, and 6.4. In some aspects, the aqueous solution with the complexing molecule has an alkaline pH. For example, as set forth in some of the working examples, solutions with rHA may have an adjusted pH to about 9.8. In some aspects, the pH of the rHA solution is greater than 10.2, or greater than 10.6.

### Methods for Preparing

In aspects, the present disclosure concerns methods for preparing the hydrogels as set forth herein. It is a particular aspect of the present disclosure that the hydrogels as set forth herein can form in situ within a subject. In other words, a user, such as a medical professional, can prepare the hydrogel solution and have the gel form from the point of application directly within a desired cavity or space in a subject. Such malleability in application allows for the hydrogel to properly occupy a desired space or cavity within a subject and the gel form therein to occupy the space.

In some aspects, the hydrogel forms in a matter of seconds to minutes. As set forth in the working examples, the hydrogels of the present disclosure offer some quick forming times. As such, it is to be considered how the hydrogel solution is prepared and applied to ensure that the hydrogel forms where desired. It is also to be considered that the hydrogel forms rapidly to provide a minimal amount of invasive time within a subject's body.

The methods of preparation therefore involve the formation of two separate solutions or components. A first solution is formed by combining a first aqueous solution with the monomeric unit therein. For example, the first solution can be formed by adding water to PEG, PEG-SS, PEG-SG, PEG-SCM, PEG-TIBA, NHS-PEG-SS-2, NHS-PEG-SS-4, NHS-PEG-SS-8, NHS-PEG-SG-2, NHS-PEG-SG-4, NHS-PEG-SG-8, NHS-PEG-SCM-2, NHS-PEG-SCM-4, NHS-PEG-SCM-8, TIBA-PEG-SS-2, TIBA-PEG SG-2, TIBA-PEG-SCM-2, TIBA-PEG-SS-4, TIBA-PEG-SG-4, TIBA-PEG-SCM-4, TIBA-PEG-SS-8, TIBA-PEG-SG-8, TIBA-PEG-SCM-8, and combinations thereof.

A second solution is formed by mixing a second aqueous solution with the complexing molecule. The second aqueous solution may be different or the same as the first aqueous solution. For example, the second solution may be formed by mixing water with PEI, rHA, poly-lysine, or similar as discussed herein or any combination thereof.

In some aspects, the first and/or second solution contain additional soluble materials therein, such as a hydrophilic therapeutic agent, contrast agent, surfactant, or similar as described herein. In some aspects, the soluble materials are pre-dissolved in the first and/or second aqueous solutions. In other aspects, the additional soluble material(s) dissolve when the first aqueous solution is added to the monomer to form a hydrated monomeric solution and/or the second aqueous solution is added to the complexing molecule to form a hydrated complexing molecule solution.

In aspects, the hydrogel will start to form once the two solutions, a hydrated monomeric solution and a hydrated complexing molecule solution, come into contact with each other. As such, a facet of preparing the hydrogels is to allow the gel to form as the solutions fill the space. An aspect of the methods of preparing the hydrogels therefore is to contact the two solutions during application. One such means by which such can be achieved is through a “Y” or “T” connection of two inputs and one output. For example, a syringe or a pump can flow the two solutions to a contact point with the output channel ending in or above the space to be filled with the hydrogel.

The two-solution hydrogel chemistry requires these two components to remain isolated from each other before use. Such may require or necessitate separate packaging. The materials can also be sensitive to moisture and may require isolation or separate packaging for long-term storage. Prior to preparing the gel, reconstitution of the dry components such as PEG, rHSA, PEI, and the PLGA particles is necessary before proceeding.

Hydrogel preparation in a clinical/surgical setting starts with hydration of the two main components, i.e. the monomeric unit and the complexing molecule. Each component is separately hydrated. For example, each can be hydrated individually by connecting a first syringe with an aqueous solution and a second syringe with dry materials therein and pushing the aqueous solution back and forth through the connector. A parallel design with dual chamber syringes can be used to hydrate the two separate parts (i.e. the monomeric unit and the complexing molecule) at the same time through a connector using similar manual process. In aspects where microparticles are to be included in the hydrogels, such mixing effectively re-suspends them therein. Alternatively, other hydration techniques can be used as well. For example, a magnetic disc or bar can be preloaded in a syringe and once the aqueous solution is introduced and capped, it can be hydrated by positioning the syringes on the magnetic stirring base and allow the magnetic disc or bar to stir and mix or hydrate. In aspects where microparticles are to be included in the hydrogels, such stirring effectively re-suspends them therein. Alternatively, hydration process can be achieved by placing syringes in an ultrasonic and/or mechanical vibrating system such as a vortex mixer. A fixture can hold the syringes in place during vortex mixing/hydration. In aspects where microparticles are to be included in the hydrogels, such applied vibrations effectively re-suspends them therein. In some aspects, the solution(s) may include two or more inert sphere therein that do not dissolve or react with any component therein, such as stainless steel or a polymer. The presence of such within the solution can assist with the reconstitution or dissolving of the components therein. In aspects where the solutions are prepare in a syringe barrel, such spheres can assist to ensure the solution is sufficiently prepared, such as through shaking, prior to mixing with the other solution to provide the hydrogel. For example, the two solutions are prepared by reconstituting dried massed amounts of each in the barrel of a syringe. In some aspects, a double-barreled syringe may be used. In some aspects, the two barrels use a needle that is configured to allow the two solutions to mix as they reach or near the needle end, such that the two solutions mix at or near the point of application. The syringe barrel can be manually shaken and the spheres therein will assist to ensure the solution is ready, while also providing an anti-foaming aspect.

After the two components are fully hydrated, the mixing and delivery of the hydrogel follows. The mixing process is critical due to the system's fast crosslinking speed (short gel time). Premature mixing before delivery could result in clogged needle while not enough mixing could lead to failed integrity of the gel structure and shape during its service life.

The mixing of the two components can be achieved during the fast flow through a long needle and further mixing by turbulence created after exiting the delivery needle tip. The mixing can be also assisted with BD Progel delivery system where the mixing is achieved by spray at the delivery tip. Alternatively, the mixing can be further assisted with a torturous path mixing section which can be positioned immediately at the end of the Y-joint or a T-joint. The delivery needle can be selected to a desired length suited for targeted application.

Once the mixing and delivery process starts, the process should proceed continuously until completion. The delivery process cannot be stopped or paused because the quick reaction gel will clog the system. In aspects where hydrogels are to be layered, the establishment of a subsequent layer may commence upon completion or near completion of the prior layer completing the gelling process. Application of a subsequent layer prior to completion of the gelling of a prior layer will allow the interface between the two layers to diffuse together, as well as allow the two layers to appear as a single hydrogel while providing the functionality of two or more layers.

### Tunability

In aspects, the hydrogels of the present disclosure can be set to achieve a desired rate of formation and/or elastic modulus and/or rheology and/or viscoelasticity. Factors such as the monomeric unit(s) selected, the concentration of the selected monomeric unit(s), the number of arms and/or the length of the linker, the complexing molecule(s) or macromolecule(s) selected, the concentration of the complexing molecule(s) or macromolecule(s) selected, the degree of crosslinking, the available side chains for cross-linking, the amount of water included or made available, and so forth. As set forth in the working examples, varying the concentrations and the MW can affect the gelling time and/or the degradation time.

As set forth in the working examples, providing a monomeric unit:complexing molecule mass ratio at about 33:1.2, 33:1.1, 33:1.4, 33:1.5, 33:1.3, 33:1.6, 33:1.7, 33:1.8, 33:1.9, or 33:2 provides a hydrogel with a degradation time of over 18 months. In some aspects, the monomeric unit is of 2, 4, or 8 arms with PEG as the linker, with a terminal structure of NHS-SG, SS, or SCM. In some aspects, the monomeric unit is of from about 2 kDa to about 40 kDa in MW. In some aspects, the complexing molecule is PEI and/or rHA.

In aspects of the present disclosure, the hydrogel forms, at least in part, through reactions between the oxygen linking the linker to the terminal group and primary amines in the complexing molecule. For example, NHS-PEG-SG-8 will react with the imines in PEI or primary amines available in the side chains of the amino acids of rHA, such as lysine, arginine, asparagine, and glutamine. In some aspects, the ester (also referred to as NHS-ester or NHS) to amine molar ratio determines certain qualities of the hydrogel. In some aspects, the ester:amine (or NHS:amine) molar ratio is between 0.05 and 3, including 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, and 3.00.

In some aspects the ester/NHS:amine molar ratio is over 1.00. In some aspects the ester/NHS:amine molar ratio is of about 1.5 to about 2.75, of about 1.5 to about 2.65, of about 1.5 to about 2.55, of about 1.5 to about 2.5, of about 1.5 to about 2.4, of about 1.5 to about 2.3, of about 1.5 to about 2.2, of about 1.5 to about 2.1, of about 1.5 to about 2.0, of about 1.5 to about 1.9, of about 1.5 to about 1.8, of about 1.5 to about 1.7, of about 1.5 to about 1.6, of about 1.6 to about 2.75, of about 1.6 to about 2.65, of about 1.6 to about 2.55, of about 1.6 to about 2.5, of about 1.6 to about 2.4, of about 1.6 to about 2.3, of about 1.6 to about 2.2, of about 1.6 to about 2.1, of about 1.6 to about 2.0, of about 1.6 to about 1.9, of about 1.6 to about 1.8, of about 1.6 to about 1.7, of about 1.7 to about 2.75, of about 1.7 to about 2.65, of about 1.7 to about 2.55, of about 1.7 to about 2.5, of about 1.7 to about 2.4, of about 1.7 to about 2.3, of about 1.7 to about 2.2, of about 1.7 to about 2.1, of about 1.7 to about 2.0, of about 1.7 to about 1.9, of about 1.7 to about 1.8, of about 1.8 to about 2.75, of about 1.8 to about 2.65, of about 1.8 to about 2.55, of about 1.8 to about 2.5, of about 1.8 to about 2.4, of about 1.8 to about 2.3, of about 1.8 to about 2.2, of about 1.8 to about 2.1, of about 1.8 to about 2.0, of about 1.8 to about 1.9, of about 1.9 to about 2.75, of about 1.9 to about 2.65, of about 1.9 to about 2.55, of about 1.9 to about 2.5, of about 1.9 to about 2.4, of about 1.9 to about 2.3, of about 1.9 to about 2.2, of about 1.9 to about 2.1, of about 1.9 to about 2.0, of about 2.0 to about 2.75, of about 2.0 to about 2.65, of about 2.0 to about 2.55, of about 2.0 to about 2.5, of about 2.0 to about 2.4, of about 2.0 to about 2.3, of about 2.0 to about 2.2, of about 2.0 to about 2.1, of about 2.1 to about 2.75, of about 2.1 to about 2.65, of about 2.1 to about 2.55, of about 2.1 to about 2.5, of about 2.1 to about 2.4, of about 2.1 to about 2.3, of about 2.1 to about 2.2, of about 2.2 to about 2.75, of about 2.2 to about 2.65, of about 2.2 to about 2.55, of about 2.2 to about 2.5, of about 2.2 to about 2.4, of about 2.2 to about 2.3, of about 2.3 to about 2.75, of about 2.3 to about 2.65, of about 2.3 to about 2.55, of about 2.3 to about 2.5, of about 2.3 to about 2.4, of about 2.4 to about 2.75, of about 2.4 to about 2.65, of about 2.4 to about 2.55, of about 2.4 to about 2.5, of about 2.5 to about 2.75, of about 2.5 to about 2.65, of about 2.5 to about 2.55, of about 2.6 to about 2.75, and of about 2.6 to about 2.65.

In some aspects, the hydrogel is of a monomeric unit of PEG-SG, -SS, or SCM at a MW of from about 10 kDa to about 20 kDa. In some aspects, the monomeric unit is added to a solution of from about 10 to about 20 wt. %/volume complexing unit, such as rHA or PEI. In some aspects, the molar ratio of ester/NHS:amine is of about 1.3 to about 2.75.

In some aspects, the hydrogel may further tuned by changing the selection of the monomeric unit and/or the complexing molecule. For example, changing from SCM to SS will allow for a hydrogel with a faster degradation time. Similarly, a change from PEI to rHA may allow for a hydrogel with a faster degradation time. Accordingly, in addition to varying the amount/concentration of each component, varying the type of component allows for additional levels of tunability.

### Layered Hydrogels

As set forth in the methods for preparing the hydrogels, two separate solutions are needed, with each solution being prepared with a step of hydrating a dried monomeric unit and a dried complexing molecule. It is a further aspect of the present disclosure that the hydrogel can include a plurality of layers such that each layer can be independently formulated, tuned, and/or loaded, as discussed in further detail herein. In aspects, the present disclosure concerns methods for preparing layered hydrogels as set forth herein. In aspects, layered hydrogels increase therapeutic effects of the targeted tissue while maintaining and protecting the non-targeted tissue

In some aspects, the method of making layered hydrogel includes the steps of making a first hydrogel layer in accordance with the preparation, mixing, and delivery steps as already described herein. It is a particular aspect of the present disclosure that the layered hydrogels as set forth herein can form in situ within a subject. It will be apparent that each layer of the plurality of layers in the layered hydrogel can form in situ within a subject. In aspects, the method further includes making one or more subsequent layers in accordance with the preparation, mixing, and delivery steps as already described herein such that the one or more subsequent layers are delivered on top of the first hydrogel layer. In some aspects, the layered hydrogel includes 2, 3, 4, 5, 6, 7, 8, 9, 10, or more layers.

In aspects, each layer of the plurality of layers can have a different formulation. For example, each layer may have an independently selected monomeric unit and/or complexing molecule and/or hydrating aqueous solution. In aspects, each layer of the plurality of layers has the same monomeric unit, complexing molecule and hydrating aqueous solution. In aspects, each layer of the plurality of layers has the same monomeric unit, complexing molecule and hydrating aqueous solution, but have one or more additional components that are independently selected from the other layers in the plurality of layers. In aspects, the plurality of layers are applied in alternating formulations.

In aspects, one or more layers of the plurality of layers are formulated such that they are protective layers and provide a protective effect to the adjacent tissue. For example, the protective layer(s) may be adjacent to the non-targeted tissue and provide radiation shielding, osmotic balance, and the like as described in greater detail herein.

In aspects, one or more layers of the plurality of layers are formulated such that they are therapeutic layers and demonstrate a therapeutic impact on the adjacent tissue. For example, the therapeutic layer(s) may be adjacent to the targeted tissue and provide radiation enhancement, osmotic imbalance, pharmaceuticals, and the like.

An aspect of the present disclosure includes a layered hydrogel having two or more distinct layers, of which a first layer protects the non-targeted tissue and a second layer treats and/or enhances a treatment of the targeted tissue. In aspects, the two layers are joined through chemical bond or an attraction, such as a covalent bond, an ionic bond, a metallic bond, van der Waal forces, hydrogen bonding, Keesome forces, Coulombic interactions, Pauli repulsions, halogen bonds, and combinations thereof.

In aspects, the layered hydrogel may be combined with other aspects of the hydrogels described herein.

### Osmotic Hydrogels

As set forth in the methods for preparing the hydrogels, two separate solutions are needed, with each solution being prepared with a step of hydrating a dried monomeric unit and a dried complexing molecule. It is a further aspect of the present disclosure that the hydrogel may include one or more osmotic components therein. It will be apparent that as each of the two solutions needed to form the hydrogel requires hydration of the monomer and the complexing molecule, osmotic components can be provided either in a dry form and added with the monomeric unit and/or complexing molecule and/or provided in an aqueous form in the hydrating aqueous solution used to hydrate the monomeric unit and/or complexing molecule. It will also be appreciated that the one or more osmotic components need to be soluble in the hydrating aqueous solution, whether the one or more osmotic component are provided in a dry form or provided in an aqueous form. In some aspects, no osmotic components are added to the monomeric unit, complexing molecules or the hydrating aqueous solution.

In aspects, the hydrogel is formulated to induce osmotic stress in a targeted tissue. In aspects, the hydrogel may contain one or more osmotic components to adjust the osmotic pressure to create hypotonic, hypertonic, and/or isotonic hydrogels. In some aspects, an osmotically-imbalanced hydrogel may be implanted to create separation between a targeted tissue and a non-targeted tissue. As used herein, an “osmotically-imbalanced hydrogel” refers to a hydrogel having an osmotic pressure that is different from the targeted tissue, thereby creating an osmotic gradient between the hydrogel and the targeted tissue. In some aspects, the osmotically-imbalanced hydrogel is a hypertonic hydrogel. In other aspects, the osmotically-imbalanced hydrogel is a hypotonic hydrogel.

In some aspects, the osmotically-imbalanced hydrogel may be a layered hydrogel, described in greater detail herein, having one or more osmotically-imbalanced layers. For example, the layered hydrogel may have 2, 3, 4, 5, 6, 7, 8, 9, 10, or more layers. Each layer of the plurality of layers can be independently formulated to be hypertonic, hypotonic, or isotonic.

### Hypertonic Hydrogels

In some aspects, the present disclosure concerns a hypertonic hydrogel. In aspects, the hydrogel is formulated such that it is hypertonic with respect to the targeted tissue, allowing fluid to be drawn out of the targeted tissue. In aspects, the hypertonic hydrogel may be placed adjacent to a cancerous tumor, causing cytotoxic dehydration and triggering death of the cells in the tumor. In aspects, osmotic pressure of the targeted tissue may have an osmotic pressure greater than 300 mOsm/L. In aspects, the hypertonic hydrogel may contain one or more osmotic components in an amount sufficient to raise the osmotic pressure of the hydrogel above the targeted tissues.

It will be appreciated that the one or more osmotic components may be provided at any concentration that is sufficient to raise the osmotic pressure above 300 mOsm/L. In aspects, the hydrogel may have an osmotic pressure at about 300 mOsm/L to about 10000 mOsm/L, including about 400, 500, 600, 700, 800, 900, 1000, 1250, 1500, 1750, 2000, 2250, 2500, 2750, 3000, 3250, 3500, 3750, 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, and 10000 mOsm/L. In some aspects, the hydrogel may have an osmotic pressure in a range having endpoints defined by any two of the aforementioned values. In some aspects, the osmotic pressure is of from about 300 to about 9000 mOsm/L, from about 300 to about 8000 mOsm/L, from about 300 to about 7000 mOsm/L, from about 300 to about 6000 mOsm/L, from about 300 to about 5000 mOsm/L, from about 300 to about 4000 mOsm/L, from about 300 to about 3000 mOsm/L, from about 300 to about 2000 mOsm/L, from about 300 to about 1000 mOsm/L, from about 300 to about 500 mOsm/L, from about 300 to about 400 mOsm/L, from about 400 to about 500 mOsm/L, from about 400 to about 600 mOsm/L, from about 400 to about 700 mOsm/L, from about 400 to about 800 mOsm/L, from about 400 to about 900 mOsm/L, from about 400 to about 1000 mOsm/L, from about 1000 to about 2000 mOsm/L, from about 2000 to about 3000 mOsm/L, from about 3000 to about 4000 mOsm/L, from about 4000 to about 5000 mOsm/L, from about 5000 to about 6000 mOsm/L, from about 6000 to about 7000 mOsm/L, from about 7000 to about 8000 mOsm/L from about 8000 to about 9000 mOsm/L, or from about 9000 to about 10000 mOsm/L.

In aspects, the aqueous solution and/or the dried components may include any soluble material in a sufficient concentration to generate a hypertonic hydrogel. In some aspects, the aqueous solution and/or the dried components may include one or more salts, one or more organic acids and/or acidic salts thereof, one or more carbohydrates, one or more monohydric and/or polyhydric alcohols, one or more amino acids and/or peptides and/or proteins, one or more other biocompatible components, combinations thereof and the like.

In aspects, the hypertonic hydrogel may include one or more salts. For example, the aqueous solution and/or the dried components may include sodium chloride, calcium chloride, potassium chloride, barium sulfate, magnesium chloride, sodium bicarbonate, sodium phosphate, sodium sulfate, potassium phosphate, potassium sulfate, calcium phosphate, ammonium sulfate, barium chloride, copper sulfate, ferric chloride, ferrous sulfate, lithium chloride, magnesium sulfate, manganese sulfate, nickel sulfate, potassium carbonate, potassium bromide, potassium chloride, potassium iodide, silver nitrate, sodium bromide, sodium carbonate, sodium chlorate, sodium nitrate, sodium pyrophosphate, zinc sulfate, combinations thereof, and the like. In some aspects, the aqueous solution and/or the dried components may include one or more salts wherein the cation is an alkali metal and/or an alkali earth metal, or combinations thereof. In some aspects, the aqueous solution and/or the dried components may include one or more salts wherein the anion is a halide, a non-metal, an oxoanion, and/or an amide, or combinations thereof. In some aspects, the salt may be an acidic salt, discussed in further detail below.

In some aspects, the salt is provided in the hypertonic hydrogel at about 1% to about 60% weight/volume (w/v), including about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 38, 40, 41, 42, 43, 44, 45, 46, 47, 48, 49, 50, 51, 52, 53, 54, 55, 56, 57, 58, and 59% w/v. In some aspects, the salt is of from about 1 to about 55% w/v, from about 1 to about 50% w/v, from about 1 to about 45% w/v, from about 1 to about 40% w/v, from about 1 to about 35% w/v, from about 1 to about 30% w/v, from about 1 to about 25% w/v, from about 1 to about 23% w/v, from about 1 to about 22% w/v, from about 1 to about 21% w/v, from about 1 to about 20% w/v, from about 1 to about 19% w/v, from about 1 to about 18% w/v, from about 1 to about 17% w/v, from about 1 to about 16% w/v, from about 1 to about 15% w/v, from about 1 to about 14% w/v, from about 1 to about 13% w/v, from about 1 to about 12% w/v, from about 1 to about 11% w/v, from about 1 to about 10% w/v, from about 1 to about 9% w/v, from about 1 to about 8% w/v, from about 1 to about 7% w/v, from about 1 to about 6% w/v, from about 1 to about 5% w/v, from about 1 to about 4% w/v, from about 1 to about 3% w/v, from about 1 to about 2% w/v, from about 5 to about 60% w/v, from about 5 to about 55% w/v, from about 5 to about 50% w/v, from about 5 to about 45% w/v, from about 5 to about 35% w/v, from about 5 to about 30% w/v, from about 5 to about 25% w/v, from about 5 to about 15% w/v, from about 5 to about 10% w/v, from about 10 to about 60% w/v, from about 10 to about 55% w/v, from about 10 to about 50% w/v, from about 10 to about 45% w/v, from about 10 to about 35% w/v, from about 10 to about 30% w/v, from about 10 to about 25% w/v, from about 10 to about 20% w/v, from about 10 to about 15% w/v, from about 15 to about 60% w/v, from about 15 to about 55% w/v, from about 15 to about 50% w/v, from about 15 to about 45% w/v, from about 15 to about 40% w/v, from about 15 to about 35% w/v, from about 15 to about 30% w/v, from about 15 to about 25% w/v, from about 15 to about 20% w/v, from about 20 to about 60% w/v, from about 20 to about 55% w/v, from about 20 to about 50% w/v, from about 20 to about 45% w/v, from about 20 to about 40% w/v, from about 20 to about 35% w/v, from about 20 to about 30% w/v, from about 20 to about 25% w/v, from about 25 to about 60% w/v, from about 25 to about 55% w/v, from about 25 to about 50% w/v, from about 25 to about 45% w/v, from about 25 to about 40% w/v, from about 25 to about 35% w/v, from about 25 to about 30% w/v, from about 30 to about 60% w/v, from about 30 to about 55% w/v, from about 30 to about 50% w/v, from about 30 to about 45% w/v, from about 30 to about 40% w/v, from about 30 to about 35% w/v, from about 35 to about 60% w/v, from about 35 to about 55% w/v, from about 35 to about 50% w/v, from about 35 to about 45% w/v, from about 35 to about 40% w/v, from about 40 to about 60% w/v, from about 40 to about 55% w/v, from about 40 to about 50% w/v, from about 40 to about 45% w/v, from about 45 to about 60% w/v, from about 45 to about 55% w/v, from about 45 to about 50% w/v, from about 50 to about 60% w/v, from about 50 to about 55% w/v, or from about 55 to about 60% w/v. In some aspects, the salt is potassium chloride. In some aspects, the salt is barium sulfate.

In aspects, the hypertonic hydrogel may include one or more organic acids or acidic salts thereof. For example, the aqueous solution and/or the dried components may include acetic acid, adipic acid, ascorbic acid, citric acid, phosphoric acid, tartaric acid, malic acid, succinic acid, fumaric acid, formic acid, gluconic acid, lactic acid, glycolic acid, pyruvic acid, oxalic acid, benzoic acid, cinnamic acid, ferulic acid, butyric acid, propionic acid, gallic acid, itaconic acid, maleic acid, mandelic acid, nicotinic acid, phthalic acid, salicylic acid, shikimic acid, uric acid, and/or valeric acid, combinations thereof, and the like.

In aspects, the hypertonic hydrogel may include one or more acidic salts of the aforementioned organic acids. In some aspects, the aqueous solution and/or the dried components may include one or more acidic salts wherein the cation is an alkali metal and/or an alkali earth metal, or combinations thereof. In some aspects, the hypertonic hydrogel may include one or more acidic salts wherein the anion is a lactate, an acetate, a citrate, a malate, a pyruvate, a citrate, a phosphate, an ascorbate, a succinate, an oxalate, a gluconate, a tartrate, a carbonate, and/or combinations thereof.

In some aspects, the organic acid and/or acidic salt thereof is provided in the hypertonic hydrogel at about 0.1%-5% weight/volume (w/v), including about 0.2, 0.3, 0.4, 0.5, 0.6, 0.7, 0.8, 0.9, 1, 1.5, 2, 2.5, 3, 3.5, 4, and 4.5% w/v. In some aspects, the organic acid and/or organic salt thereof is of from about 0.1 to about 4.5% w/v, from about 0.1 to about 4% w/v, from about 0.1 to about 3.5% w/v, from about 0.1 to about 3% w/v, from about 0.1 to about 2.5% w/v, from about 0.1 to about 2% w/v, from about 0.1 to about 1.5% w/v, from about 0.1 to about 1% w/v, from about 0.5 to about 5% w/v, from about 0.5 to about 4.5% w/v, from about 0.5 to about 4% w/v, from about 0.5 to about 3.5% w/v, from about 0.5 to about 3% w/v, from about 0.5 to about 2.5% w/v, from about 0.5 to about 2% w/v, from about 0.5 to about 1.5% w/v, from about 0.5 to about 1% w/v, from about 1 to about 5% w/v, from about 1 to about 4.5% w/v, from about 1 to about 4% w/v, from about 1 to about 3.5% w/v, from about 1 to about 3% w/v, from about 1 to about 2.5% w/v, from about 1 to about 2% w/v, from about 1 to about 1.5% w/v, from about 1.5 to about 2% w/v, from about 1.5 to about 2.5% w/v, or from about 1.5 to about 5% w/v.

In aspects, the hypertonic hydrogel may include one or more carbohydrates. For example, the aqueous solution and/or the dried components may include dextrose, fructose, sucrose, lactose, maltose, trehalose, galactose, dextran, xylose, mannose, ribose, isomaltase, inulin, cyclodextrin, hydroxyethyl starch (HES), pullulan, pectin, xanthan gum, agarose, cellulose, hyaluronic acid, maltodextrin, methylcellulose, alginate, chitosan, heparin, and/or heparan sulfate, combinations thereof, and the like.

In some aspects, the carbohydrate is provided in the hypertonic hydrogel at about 1-20% weight/volume (w/v), including about 2, 3, 4, 5, 6, 7, 8, 9, 10, 11, 12, 13, 14, 15, 16, 17, 18, and 19% w/v. In some aspects, the carbohydrate is of from about 1 to about 19% w/v, from about 2 to about 18% w/v, from about 3 to about 17% w/v, from about 4 to about 16% w/v, from about 5 to about 19% w/v, from about 5 to about 18% w/v, from about 5 to about 17% w/v, from about 5 to about 16% w/v, from about 5 to about 15% w/v, from about 5 to about 14% w/v, from about 5 to about 13% w/v, from about 5 to about 12% w/v, from about 5 to about 11% w/v, from about 5 to about 10% w/v, from about 6 to about 10% w/v, from about 7 to about 10% w/v, from about 8 to about 10% w/v, from about 9 to about 10% w/v, from about 10 to about 20% w/v, from about 10 to about 19% w/v, from about 10 to about 18% w/v, from about 10 to about 17% w/v, from about 10 to about 16% w/v, from about 10 to about 15% w/v, from about 10 to about 14% w/v, from about 10 to about 13% w/v, or from about 10 to about 11% w/v.

In aspects, the hypertonic hydrogel may include one or more monohydric and/or polyhydric alcohols. For example, the aqueous solution and/or the dried components may include ethanol, 1-propanol, propylene glycol, ethylene glycol, xylitol, sorbitol, erythritol, mannitol, isomalt, maltitol, lactitol, arabitol, ribitol, glycerol, threitol, dulcitol, inositol, iditiol, adonitol, and/or galactitol, combinations thereof, and the like.

In some aspects, the monohydric and/or polyhydric alcohol is provided in the hypertonic hydrogel at about 1-40% weight/volume (w/v), including about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39% w/v. In some aspects, the carbohydrate is of from about 1 to about 35% w/v, from about 2 to about 30% w/v, from about 3 to about 25% w/v, from about 4 to about 20% w/v, from about 5 to about 10% w/v, from about 5 to about 15% w/v, from about 5 to about 20% w/v, from about 5 to about 25% w/v, from about 5 to about 30% w/v, from about 5 to about 35% w/v, from about 5 to about 40% w/v, from about 10 to about 15% w/v, from about 10 to about 20% w/v, from about 10 to about 25% w/v, from about 10 to about 30% w/v, from about 10 to about 35% w/v, from about 10 to about 40% w/v, from about 15 to about 20% w/v, from about 15 to about 25% w/v, from about 15 to about 30% w/v, from about 15 to about 35% w/v, from about 15 to about 40% w/v, from about 20 to about 25% w/v, from about 20 to about 30% w/v, from about 20 to about 35% w/v, from about 20 to about 40% w/v, from about 25 to about 30% w/v, from about 25 to about 35% w/v, from about 25 to about 40% w/v, from about 30 to about 35% w/v, from about 30 to about 40% w/v, or from about 35% to about 40% w/v.

In aspects, the hypertonic hydrogel may include one or more amino acids. For example, the aqueous solution and/or the dried components may include proline, proline analogs (e.g., hydroxyproline, pipecolic acid, azetidine-2-carboxylic acid), glycine, aspartic acid, taurine, glutamate, alanine, histidine, serine, arginine, and/or lysine, combinations thereof and the like. In some aspects, the aqueous solution and/or the dried components may include polyamino acids of the aforementioned amino acids, such as polyproline, polylysine, polyarginine, polyglutamate, polyhistidine, combinations thereof, and the like. In some aspects, the aqueous solution and/or the dried components may include peptides and/or polypeptides rich in the aforementioned amino acids. For example, the aqueous solution and/or the dried components may include proline-rich peptides, such as dehydrins, collagen, proline-rich salivary glycoproteins, combinations thereof and the like. In some aspects, the aqueous solution and/or the dried components may include one or more peptides such as glutathione, carnosine, betaine, anserine, leucine-enkephalin, oxytocin, vasopressin, melanin-concentrating hormone, combinations thereof, and the like.

In aspects, the amino acid and/or peptide and/or protein is provided in the hypertonic hydrogel at about 1-40% weight/volume (w/v), including about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39% w/v. In some aspects, the carbohydrate is of from about 1 to about 35% w/v, from about 2 to about 30% w/v, from about 3 to about 25% w/v, from about 4 to about 20% w/v, from about 5 to about 10% w/v, from about 5 to about 15% w/v, from about 5 to about 20% w/v, from about 5 to about 25% w/v, from about 5 to about 30% w/v, from about 5 to about 35% w/v, from about 5 to about 40% w/v, from about 10 to about 15% w/v, from about 10 to about 20% w/v, from about 10 to about 25% w/v, from about 10 to about 30% w/v, from about 10 to about 35% w/v, from about 10 to about 40% w/v, from about 15 to about 20% w/v, from about 15 to about 25% w/v, from about 15 to about 30% w/v, from about 15 to about 35% w/v, from about 15 to about 40% w/v, from about 20 to about 25% w/v, from about 20 to about 30% w/v, from about 20 to about 35% w/v, from about 20 to about 40% w/v, from about 25 to about 30% w/v, from about 25 to about 35% w/v, from about 25 to about 40% w/v, from about 30 to about 35% w/v, from about 30 to about 40% w/v, or from about 35% to about 40% w/v.

In aspects, the hypertonic hydrogel may include one or more other biocompatible compounds. For example, the aqueous solution and/or the dried components may include one or more buffers, such as tris(hydroxymethyl)aminomethane (TRIS), bis-tris, tris-hydrochloride, N-2-hydroxyethylpiperazine-N-2-ethane sulfonic acid (HEPES), 3-(N-morpholino) propanesulfonic acid (MOPS), 2-(N-morpholino) ethanesulfonic acid (MES), bicine, N-(2-hydroxyethyl) piperazine (HEPPSO), tris(hydroxymethyl)methylamino propanesulfonic acid (TAPS), and/or piperazine-N,N′-bis(2-ethanesulfonic acid) (PIPES), combinations thereof, and the like; one or more water-soluble polymers, such as polyethylene glycol, polyvinylpyrrolidone, polyethyleneimine, polyacrylic acid, polyvinyl alcohol, sodium hyaluronate, and the like; one or more quaternary ammonium compounds, such as choline chloride, trimethylamine N-oxide, proline betaine, dimethylsulfoniopropionate, and/or sarcosine betaine, combinations thereof, and the like; and/or one or more surfactants, such as polysorbate 80, polysorbate 20, cetyltrimethylammonium bromide, sodium dodecyl sulfate, poloxamers, triton X-100, sodium deoxycholate, and/or 3-[(3-cholamidopropyl)dimethylammonio]-1-propanesulfonate (CHAPS), combinations thereof, and the like. It will be appreciated that the one or more biocompatible compounds may be provided at any concentration that is sufficient to generate the desired osmotic pressure.

In some aspects, the hypertonic hydrogel may include one or more salts in combination with one or more organic acids and/or acidic salts thereof. In some aspects, the hypertonic hydrogel may include one or more salts in combination with one or more carbohydrates. In some aspects, the hypertonic hydrogel may include one or more salts in combination with one or more organic acids and/or acidic salts thereof and/or one or more carbohydrates. In some aspects, the hypertonic hydrogel may include one or more salts in combination with one or more monohydric and/or polyhydric alcohols. In some aspects, the hypertonic hydrogel may include one or more salts in combination with one or more amino acids and/or peptides and/or proteins. In some aspects, the hypertonic hydrogel may include one or more salts in combination with one or more other biocompatible compounds. In some aspects, the hypertonic hydrogel may include one or more salts in combination with one or more organic acids and/or acidic salts thereof, one or more carbohydrates, and/or one or more monohydric and/or polyhydric alcohols. In some aspects, the hypertonic hydrogel may include one or more salts in combination with one or more organic acids and/or acidic salts thereof, one or more carbohydrates, one or more monohydric and/or polyhydric alcohols, and/or one or more amino acids and/or peptides and/or proteins. In some aspects, the hypertonic hydrogel may include one or more salts in combination with one or more organic acids and/or acidic salts thereof, one or more carbohydrates, one or more monohydric and/or polyhydric alcohols, one or more amino acids and/or one or more other biocompatible compounds.

It will be appreciated that the concentration of the one or more additional materials may vary based on the formulation of the hypertonic hydrogel. For example, if a hypertonic hydrogel is formulated with one or more salts and one or more carbohydrates, the hypertonic solution may contain a lower concentration of the one or more salts compared with a hypertonic solution formulated with only one or more salts but having similar osmotic pressures.

### Hypotonic Hydrogels

In some aspects, the present disclosure concerns a hypotonic hydrogel. In aspects, the hydrogel is formulated such that it is hypotonic with respect to the targeted tissue, allowing fluid to be drawn into the targeted tissue, leading to lysis of the cells. In aspects, the hypotonic hydrogel may be placed adjacent to a cancerous tumor. In aspects, the hydrogel may be formulated such that the osmotic pressure of the hydrogel is below 280 mOsm/L.

In aspects, the hypotonic hydrogel may have an osmotic pressure at about 0 mOsm/L to about 280 mOsm/L, including about 20, 40, 50, 60, 80, 100, 120, 140, 150, 160, 180, 200, 220, 240, 250, and 260 mOsm/L. In some aspects, the hydrogel may have an osmotic pressure in a range having endpoints defined by any two of the aforementioned values. In some aspects, the osmotic pressure is of from about 0 to about 260 mOsm/L, from about 0 to about 250 mOsm/L, from about 0 to about 240 mOsm/L, from about 0 to about 220 mOsm/L, from about 0 to about 200 mOsm/L, from about 0 to about 180 mOsm/L, from about 0 to about 160 mOsm/L, from about 0 to about 150 mOsm/L, from about 0 to about 140 mOsm/L, from about 0 to about 120 mOsm/L, from about 0 to about 100 mOsm/L, from about 0 to about 80 mOsm/L, from about 0 to about 60 mOsm/L, from about 0 to about 50 mOsm/L, from about 0 to about 40 mOsm/L, from about 0 to about 20 mOsm/L, from about 20 to about 280 mOsm/L, from about 20 to about 260 mOsm/L, from about 20 to about 250 mOsm/L, from about 20 to about 240 mOsm/L, from about 20 to about 220 mOsm/L, from about 20 to about 200 mOsm/L, from about 20 to about 180 mOsm/L, from about 20 to about 160 mOsm/L, from about 20 to about 150 mOsm/L, from about 20 to about 140 mOsm/L, from about 20 to about 120 mOsm/L, from about 20 to about 100 mOsm/L, from about 20 to about 80 mOsm/L, from about 20 to about 60 mOsm/L, from about 20 to about 50 mOsm/L, from about 20 to about 40 mOsm/L, from about 40 to about 280 mOsm/L, from about 40 to about 260 mOsm/L, from about 40 to about 250 mOsm/L, from about 40 to about 240 mOsm/L, from about 40 to about 220 mOsm/L, from about 40 to about 200 mOsm/L, from about 40 to about 180 mOsm/L, from about 40 to about 160 mOsm/L, from about 40 to about 150 mOsm/L, from about 40 to about 140 mOsm/L, from about 40 to about 120 mOsm/L, from about 40 to about 100 mOsm/L, from about 40 to about 80 mOsm/L, from about 40 to about 60 mOsm/L, from about 40 to about 50 mOsm/L, from about 50 to about 280 mOsm/L, from about 50 to about 250 mOsm/L, from about 50 to about 200 mOsm/L, from about 50 to about 150 mOsm/L, from about 50 to about 100 mOsm/L, from about 60 to about 280 mOsm/L, from about 60 to about 260 mOsm/L, from about 60 to about 250 mOsm/L, from about 60 to about 240 mOsm/L, from about 60 to about 220 mOsm/L, from about 60 to about 200 mOsm/L, from about 60 to about 180 mOsm/L, from about 60 to about 160 mOsm/L, from about 60 to about 150 mOsm/L, from about 60 to about 140 mOsm/L, from about 60 to about 120 mOsm/L, from about 60 to about 100 mOsm/L, from about 60 to about 80 mOsm/L, from about 80 to about 280 mOsm/L, from about 80 to about 260 mOsm/L, from about 80 to about 250 mOsm/L, from about 80 to about 240 mOsm/L, from about 80 to about 220 mOsm/L, from about 80 to about 200 mOsm/L, from about 80 to about 180 mOsm/L, from about 80 to about 160 mOsm/L, from about 80 to about 150 mOsm/L, from about 80 to about 140 mOsm/L, from about 80 to about 120 mOsm/L, from about 80 to about 100 mOsm/L, from about 100 to about 280 mOsm/L, from about 100 to about 260 mOsm/L, from about 100 to about 250 mOsm/L, from about 100 to about 240 mOsm/L, from about 100 to about 220 mOsm/L, from about 100 to about 200 mOsm/L, from about 100 to about 180 mOsm/L, from about 100 to about 160 mOsm/L, from about 100 to about 150 mOsm/L, from about 100 to about 140 mOsm/L, from about 100 to about 120 mOsm/L, from about 120 to about 280 mOsm/L, from about 120 to about 260 mOsm/L, from about 120 to about 250 mOsm/L, from about 120 to about 240 mOsm/L, from about 120 to about 220 mOsm/L, from about 120 to about 200 mOsm/L, from about 120 to about 180 mOsm/L, from about 120 to about 160 mOsm/L, from about 120 to about 150 mOsm/L, from about 120 to about 140 mOsm/L, from about 140 to about 280 mOsm/L, from about 140 to about 260 mOsm/L, from about 140 to about 250 mOsm/L, from about 140 to about 240 mOsm/L, from about 140 to about 220 mOsm/L, from about 140 to about 200 mOsm/L, from about 140 to about 180 mOsm/L, from about 140 to about 160 mOsm/L, from about 140 to about 150 mOsm/L, from about 150 mOsm/L to about 280 mOsm/L, from about 150 mOsm/L to about 250 mOsm/L, from about 150 mOsm/L to about 200 mOsm/L, from about 150 mOsm/L to about 180 mOsm/L, from about 160 to about 280 mOsm/L, from about 160 to about 260 mOsm/L, from about 160 to about 250 mOsm/L, from about 160 to about 240 mOsm/L, from about 160 to about 220 mOsm/L, from about 160 to about 200 mOsm/L, from about 160 to about 180 mOsm/L, from about 180 to about 280 mOsm/L, from about 180 to about 260 mOsm/L, from about 180 to about 250 mOsm/L, from about 180 to about 240 mOsm/L, from about 180 to about 220 mOsm/L, from about 180 to about 200 mOsm/L, from about 200 to about 280 mOsm/L, from about 200 to about 260 mOsm/L, from about 200 to about 250 mOsm/L, from about 200 to about 240 mOsm/L, from about 200 to about 220 mOsm/L, from about 220 to about 280 mOsm/L, from about 220 to about 260 mOsm/L, from about 220 to about 250 mOsm/L, from about 220 to about 240 mOsm/L, from about 240 to about 280 mOsm/L, from about 240 to about 260 mOsm/L, from about 240 to about 250 mOsm/L, from about 250 to about 280 mOsm/L, from about 250 to about 260 mOsm/L or from about 260 mOsm/L to about 280 mOsm/L.

In aspects, no osmotic components are added to the monomeric unit, complexing molecules or the hydrating aqueous solution. In some aspects, the hypotonic hydrogel is formulated using one or more hypotonic hydrating aqueous solutions, such as distilled water, reverse osmosis water, ethanol, hypotonic saline, dextrose, combinations thereof and the like. In some aspects, the hypotonic hydrogel contains only the monomeric unit, the complexing molecule and the hydrating aqueous solution.

In aspects, the aqueous solution and/or the dried components may include any soluble material in a sufficient concentration to generate a hypotonic hydrogel. In some aspects, the aqueous solution and/or the dried components may include one or more salts, one or more organic acids and/or acidic salts thereof, one or more carbohydrates, one or more monohydric and/or polyhydric alcohols, one or more amino acids and/or peptides and/or proteins, one or more other biocompatible components, combinations thereof, and the like. It will be appreciated that the one or more osmotic components can be provided in any concentration or combination that results in a hypotonic hydrogel having an osmotic pressure less than 280 mOsm/L.

In aspects, the hypotonic hydrogel may include one or more salts. For example, the aqueous solution and/or the dried components may include sodium chloride, calcium chloride, potassium chloride, barium sulfate, magnesium chloride, sodium bicarbonate, sodium phosphate, sodium sulfate, potassium phosphate, potassium sulfate, calcium phosphate, ammonium sulfate, barium chloride, copper sulfate, ferric chloride, ferrous sulfate, lithium chloride, magnesium sulfate, manganese sulfate, nickel sulfate, potassium carbonate, potassium bromide, potassium chloride, potassium iodide, silver nitrate, sodium bromide, sodium carbonate, sodium chlorate, sodium nitrate, sodium pyrophosphate, zinc sulfate, combinations thereof, and the like. In some aspects, the aqueous solution and/or the dried components may include one or more salts wherein the cation is an alkali metal and/or an alkali earth metal, or combinations thereof. In some aspects, the aqueous solution and/or the dried components may include one or more salts wherein the anion is a halide, a non-metal, an oxoanion, and/or an amide, or combinations thereof. In some aspects, the salt may be an acidic salt, discussed in further detail below.

In aspects, the hypotonic hydrogel may include one or more organic acids or acidic salts thereof. For example, the aqueous solution and/or the dried components may include acetic acid, adipic acid, ascorbic acid, citric acid, phosphoric acid, tartaric acid, malic acid, succinic acid, fumaric acid, formic acid, gluconic acid, lactic acid, glycolic acid, pyruvic acid, oxalic acid, benzoic acid, cinnamic acid, ferulic acid, butyric acid, propionic acid, gallic acid, itaconic acid, maleic acid, mandelic acid, nicotinic acid, phthalic acid, salicylic acid, shikimic acid, uric acid, and/or valeric acid, combinations thereof, and the like.

In aspects, the hypotonic hydrogel may include one or more acidic salts of the aforementioned organic acids. In some aspects, the aqueous solution and/or the dried components may include one or more acidic salts wherein the cation is an alkali metal and/or an alkali earth metal, or combinations thereof. In some aspects, the hypertonic hydrogel may include one or more acidic salts wherein the anion is a lactate, an acetate, a citrate, a malate, a pyruvate, a citrate, a phosphate, an ascorbate, a succinate, an oxalate, a gluconate, a tartrate, a carbonate, and/or combinations thereof.

In aspects, the hypotonic hydrogel may include one or more carbohydrates. For example, the aqueous solution and/or the dried components may include dextrose, fructose, sucrose, lactose, maltose, trehalose, galactose, dextran, xylose, mannose, ribose, isomaltase, inulin, cyclodextrin, hydroxyethyl starch (HES), pullulan, pectin, xanthan gum, agarose, cellulose, hyaluronic acid, maltodextrin, methylcellulose, alginate, chitosan, heparin, and/or heparan sulfate, combinations thereof, and the like.

In aspects, the hypotonic hydrogel may include one or more monohydric and/or polyhydric alcohols. For example, the aqueous solution and/or the dried components may include ethanol, 1-propanol, propylene glycol, ethylene glycol, xylitol, sorbitol, erythritol, mannitol, isomalt, maltitol, lactitol, arabitol, ribitol, glycerol, threitol, dulcitol, inositol, iditiol, adonitol, and/or galactitol, combinations thereof, and the like.

In aspects, the hypotonic hydrogel may include one or more amino acids. For example, the aqueous solution and/or the dried components may include glutamic acid, aspartic acid, cysteine, tyrosine, taurine, histidine, arginine, and/or lysine, combinations thereof and the like. In some aspects, the aqueous solution and/or the dried components may include polyamino acids of the aforementioned amino acids, such as, but not limited to, polylysine, polyarginine, polyglutamate, polyhistidine, combinations thereof, and the like. In some aspects, the aqueous solution and/or the dried components may include peptides and/or polypeptides rich in the aforementioned amino acids.

In aspects, the hypotonic hydrogel may include one or more other biocompatible compounds. For example, the aqueous solution and/or the dried components may include one or more buffers, such as tris(hydroxymethyl)aminomethane (TRIS), bis-tris, tris-hydrochloride, N-2-hydroxyethylpiperazine-N-2-ethane sulfonic acid (HEPES), 3-(N-morpholino) propanesulfonic acid (MOPS), 2-(N-morpholino) ethanesulfonic acid (MES), bicine, N-(2-hydroxyethyl) piperazine (HEPPSO), tris(hydroxymethyl)methylamino]propanesulfonic acid (TAPS), and/or piperazine-N,N′-bis(2-ethanesulfonic acid) (PIPES), combinations thereof, and the like; one or more water-soluble polymers, such as polyethylene glycol, polyvinylpyrrolidone, polyethyleneimine, polyacrylic acid, polyvinyl alcohol, sodium hyaluronate, and the like; one or more quaternary ammonium compounds, such as choline chloride, trimethylamine N-oxide, proline betaine, dimethylsulfoniopropionate, and/or sarcosine betaine, combinations thereof, and the like; and/or one or more surfactants, such as polysorbate 80, polysorbate 20, cetyltrimethylammonium bromide, sodium dodecyl sulfate, poloxamers, triton X-100, sodium deoxycholate, and/or 3-[(3-cholamidopropyl)dimethylammonio]-1-propanesulfonate (CHAPS), combinations thereof, and the like. It will be appreciated that the one or more biocompatible compounds may be provided at any concentration that is sufficient to generate the desired osmotic pressure.

In some aspects, the hypotonic hydrogel may include one or more salts in combination with one or more organic acids and/or acidic salts thereof. In some aspects, the hypotonic hydrogel may include one or more salts in combination with one or more carbohydrates. In some aspects, the hypotonic hydrogel may include one or more salts in combination with one or more organic acids and/or acidic salts thereof and/or one or more carbohydrates. In some aspects, the hypotonic hydrogel may include one or more salts in combination with one or more monohydric and/or polyhydric alcohols. In some aspects, the hypotonic hydrogel may include one or more salts in combination with one or more amino acids and/or peptides and/or proteins. In some aspects, the hypotonic hydrogel may include one or more salts in combination with one or more other biocompatible compounds. In some aspects, the hypotonic hydrogel may include one or more salts in combination with one or more organic acids and/or acidic salts thereof, one or more carbohydrates, and/or one or more monohydric and/or polyhydric alcohols. In some aspects, the hypotonic hydrogel may include one or more salts in combination with one or more organic acids and/or acidic salts thereof, one or more carbohydrates, one or more monohydric and/or polyhydric alcohols, and/or one or more amino acids and/or peptides and/or proteins. In some aspects, the hypotonic hydrogel may include one or more salts in combination with one or more organic acids and/or acidic salts thereof, one or more carbohydrates, one or more monohydric and/or polyhydric alcohols, one or more amino acids and/or one or more other biocompatible compounds.

### Isotonic Hydrogels

In some aspects, the present disclosure concerns an isotonic hydrogel. In aspects, the hydrogel is formulated such that it is isotonic with respect to the targeted tissue and/or the non-targeted tissue, allowing for osmotic balance between the tissue and the hydrogels spacer. In aspects, the isotonic hydrogel may be placed adjacent to a non-target tissue to provide a layer of protection. In aspects, the hydrogel may be formulated such that the osmotic pressure of the hydrogel is about 290 mOsm/L.

In aspects, the isotonic hydrogel may have an osmotic pressure at about 280 mOsm/L to about 300 mOsm/L, including about 281, 282, 283, 284, 285, 286, 287, 288, 289, 290, 291, 292, 293, 294, 295, 296, 297, 298, and 299 mOsm/L. In some aspects, the hydrogel may have an osmotic pressure in a range having endpoints defined by any two of the aforementioned values. In some aspects, the osmotic pressure is of from about 280 to about 299 mOsm/L, from about 280 to about 298 mOsm/L, from about 280 to about 297 mOsm/L, from about 280 to about 296 mOsm/L, from about 280 to about 295 mOsm/L, from about 280 to about 294 mOsm/L, from about 280 to about 293 mOsm/L, from about 280 to about 292 mOsm/L, from about 280 to about 291 mOsm/L, from about 280 to about 290 mOsm/L, from about 280 to about 289 mOsm/L, from about 280 to about 288 mOsm/L, from about 280 to about 287 mOsm/L, from about 280 to about 286 mOsm/L, from about 280 to about 285 mOsm/L, from about 280 to about 284 mOsm/L, from about 280 to about 283 mOsm/L, from about 280 to about 282 mOsm/L, from about 280 to about 281 mOsm/L, from about 281 to about 299 mOsm/L, from about 281 to about 298 mOsm/L, from about 281 to about 297 mOsm/L, from about 281 to about 296 mOsm/L, from about 281 to about 295 mOsm/L, from about 281 to about 294 mOsm/L, from about 281 to about 293 mOsm/L, from about 281 to about 292 mOsm/L, from about 281 to about 291 mOsm/L, from about 281 to about 290 mOsm/L, from about 281 to about 289 mOsm/L, from about 281 to about 288 mOsm/L, from about 281 to about 287 mOsm/L, from about 281 to about 286 mOsm/L, from about 281 to about 285 mOsm/L, from about 281 to about 284 mOsm/L, from about 281 to about 283 mOsm/L, from about 281 to about 282 mOsm/L, from about 282 to about 299 mOsm/L, from about 282 to about 298 mOsm/L, from about 282 to about 297 mOsm/L, from about 282 to about 296 mOsm/L, from about 282 to about 295 mOsm/L, from about 282 to about 294 mOsm/L, from about 282 to about 293 mOsm/L, from about 282 to about 292 mOsm/L, from about 282 to about 291 mOsm/L, from about 282 to about 290 mOsm/L, from about 282 to about 289 mOsm/L, from about 282 to about 288 mOsm/L, from about 282 to about 287 mOsm/L, from about 282 to about 286 mOsm/L, from about 282 to about 285 mOsm/L, from about 282 to about 284 mOsm/L, from about 282 to about 283 mOsm/L, from about 283 to about 299 mOsm/L, from about 283 to about 298 mOsm/L, from about 283 to about 297 mOsm/L, from about 283 to about 296 mOsm/L, from about 283 to about 295 mOsm/L, from about 283 to about 294 mOsm/L, from about 283 to about 293 mOsm/L, from about 283 to about 292 mOsm/L, from about 283 to about 291 mOsm/L, from about 283 to about 290 mOsm/L, from about 283 to about 289 mOsm/L, from about 283 to about 288 mOsm/L, from about 283 to about 287 mOsm/L, from about 283 to about 286 mOsm/L, from about 283 to about 285 mOsm/L, from about 283 to about 284 mOsm/L, from about 284 to about 299 mOsm/L, from about 284 to about 298 mOsm/L, from about 284 to about 297 mOsm/L, from about 284 to about 296 mOsm/L, from about 284 to about 295 mOsm/L, from about 284 to about 294 mOsm/L, from about 284 to about 293 mOsm/L, from about 284 to about 292 mOsm/L, from about 284 to about 291 mOsm/L, from about 284 to about 290 mOsm/L, from about 284 to about 289 mOsm/L, from about 284 to about 288 mOsm/L, from about 284 to about 287 mOsm/L, from about 284 to about 286 mOsm/L, from about 284 to about 285 mOsm/L, from about 285 to about 299 mOsm/L, from about 285 to about 298 mOsm/L, from about 285 to about 297 mOsm/L, from about 285 to about 296 mOsm/L, from about 285 to about 295 mOsm/L, from about 285 to about 294 mOsm/L, from about 285 to about 293 mOsm/L, from about 285 to about 292 mOsm/L, from about 285 to about 291 mOsm/L, from about 285 to about 290 mOsm/L, from about 285 to about 289 mOsm/L, from about 285 to about 288 mOsm/L, from about 285 to about 287 mOsm/L, from about 285 to about 286 mOsm/L, from about 286 to about 299 mOsm/L, from about 286 to about 298 mOsm/L, from about 286 to about 297 mOsm/L, from about 286 to about 296 mOsm/L, from about 286 to about 295 mOsm/L, from about 286 to about 294 mOsm/L, from about 286 to about 293 mOsm/L, from about 286 to about 292 mOsm/L, from about 286 to about 291 mOsm/L, from about 286 to about 290 mOsm/L, from about 286 to about 289 mOsm/L, from about 286 to about 288 mOsm/L, from about 286 to about 287 mOsm/L, from about 287 to about 299 mOsm/L, from about 287 to about 298 mOsm/L, from about 287 to about 297 mOsm/L, from about 287 to about 296 mOsm/L, from about 287 to about 295 mOsm/L, from about 287 to about 294 mOsm/L, from about 287 to about 293 mOsm/L, from about 287 to about 292 mOsm/L, from about 287 to about 291 mOsm/L, from about 287 to about 290 mOsm/L, from about 287 to about 289 mOsm/L, from about 287 to about 288 mOsm/L, from about 288 to about 299 mOsm/L, from about 288 to about 298 mOsm/L, from about 288 to about 297 mOsm/L, from about 288 to about 296 mOsm/L, from about 288 to about 295 mOsm/L, from about 288 to about 294 mOsm/L, from about 288 to about 293 mOsm/L, from about 288 to about 292 mOsm/L, from about 288 to about 291 mOsm/L, from about 288 to about 290 mOsm/L, from about 288 to about 289 mOsm/L, from about 289 to about 299 mOsm/L, from about 289 to about 298 mOsm/L, from about 289 to about 297 mOsm/L, from about 289 to about 296 mOsm/L, from about 289 to about 295 mOsm/L, from about 289 to about 294 mOsm/L, from about 289 to about 293 mOsm/L, from about 289 to about 292 mOsm/L, from about 289 to about 291 mOsm/L, from about 289 to about 290 mOsm/L, from about 290 to about 299 mOsm/L, from about 290 to about 298 mOsm/L, from about 290 to about 297 mOsm/L, from about 290 to about 296 mOsm/L, from about 290 to about 295 mOsm/L, from about 290 to about 294 mOsm/L, from about 290 to about 293 mOsm/L, from about 290 to about 292 mOsm/L, from about 290 to about 291 mOsm/L, from about 291 to about 299 mOsm/L, from about 291 to about 298 mOsm/L, from about 291 to about 297 mOsm/L, from about 291 to about 296 mOsm/L, from about 291 to about 295 mOsm/L, from about 291 to about 294 mOsm/L, from about 291 to about 293 mOsm/L, from about 291 to about 292 mOsm/L, from about 292 to about 299 mOsm/L, from about 292 to about 298 mOsm/L, from about 292 to about 297 mOsm/L, from about 292 to about 296 mOsm/L, from about 292 to about 295 mOsm/L, from about 292 to about 294 mOsm/L, from about 292 to about 293 mOsm/L, from about 293 to about 299 mOsm/L, from about 293 to about 298 mOsm/L, from about 293 to about 297 mOsm/L, from about 293 to about 296 mOsm/L, from about 293 to about 295 mOsm/L, from about 293 to about 294 mOsm/L, from about 294 to about 299 mOsm/L, from about 294 to about 298 mOsm/L, from about 294 to about 297 mOsm/L, from about 294 to about 296 mOsm/L, from about 294 to about 295 mOsm/L, from about 295 to about 299 mOsm/L, from about 295 to about 298 mOsm/L, from about 295 to about 297 mOsm/L, from about 295 to about 296 mOsm/L, from about 296 to about 299 mOsm/L, from about 296 to about 298 mOsm/L, from about 296 to about 297 mOsm/L, from about 297 to about 299 mOsm/L, from about 297 to about 298 mOsm/L, from about 298 to about 299 mOsm/L, or from about 299 to about 300 mOsm/L.

In aspects, the aqueous solution and/or the dried components may include any soluble material in a sufficient concentration to generate an isotonic hydrogel. In some aspects, the aqueous solution and/or the dried components may include one or more salts, one or more organic acids and/or acidic salts thereof, one or more carbohydrates, one or more monohydric and/or polyhydric alcohols, one or more amino acids and/or peptides and/or proteins, one or more other biocompatible components, combinations thereof, and the like. It will be appreciated that the one or more osmotic components can be provided in any concentration and/or combinations that results in an isotonic hydrogel having an osmotic pressure between about 280 mOsm/L and 300 mOsm/L.

In aspects, the isotonic hydrogel may include one or more salts. For example, the aqueous solution and/or the dried components may include sodium chloride, calcium chloride, potassium chloride, barium sulfate, magnesium chloride, sodium bicarbonate, sodium phosphate, sodium sulfate, potassium phosphate, potassium sulfate, calcium phosphate, ammonium sulfate, barium chloride, copper sulfate, ferric chloride, ferrous sulfate, lithium chloride, magnesium sulfate, manganese sulfate, nickel sulfate, potassium carbonate, potassium bromide, potassium chloride, potassium iodide, silver nitrate, sodium bromide, sodium carbonate, sodium chlorate, sodium nitrate, sodium pyrophosphate, zinc sulfate, combinations thereof, and the like. In some aspects, the aqueous solution and/or the dried components may include one or more salts wherein the cation is an alkali metal and/or an alkali earth metal, or combinations thereof. In some aspects, the aqueous solution and/or the dried components may include one or more salts wherein the anion is a halide, a non-metal, an oxoanion, and/or an amide, or combinations thereof. In some aspects, the salt may be an acidic salt, discussed in further detail below.

In aspects, the isotonic hydrogel may include one or more organic acids or acidic salts thereof. For example, the aqueous solution and/or the dried components may include acetic acid, adipic acid, ascorbic acid, citric acid, phosphoric acid, tartaric acid, malic acid, succinic acid, fumaric acid, formic acid, gluconic acid, lactic acid, glycolic acid, pyruvic acid, oxalic acid, benzoic acid, cinnamic acid, ferulic acid, butyric acid, propionic acid, gallic acid, itaconic acid, maleic acid, mandelic acid, nicotinic acid, phthalic acid, salicylic acid, shikimic acid, uric acid, and/or valeric acid, combinations thereof, and the like.

In aspects, the isotonic hydrogel may include one or more acidic salts of the aforementioned organic acids. In some aspects, the aqueous solution and/or the dried components may include one or more acidic salts wherein the cation is an alkali metal and/or an alkali earth metal, or combinations thereof. In some aspects, the isotonic hydrogel may include one or more acidic salts wherein the anion is a lactate, an acetate, a citrate, a malate, a pyruvate, a citrate, a phosphate, an ascorbate, a succinate, an oxalate, a gluconate, a tartrate, a carbonate, and/or combinations thereof.

In aspects, the isotonic hydrogel may include one or more carbohydrates. For example, the aqueous solution and/or the dried components may include dextrose, fructose, sucrose, lactose, maltose, trehalose, galactose, dextran, xylose, mannose, ribose, isomaltase, inulin, cyclodextrin, hydroxyethyl starch (HES), pullulan, pectin, xanthan gum, agarose, cellulose, hyaluronic acid, maltodextrin, methylcellulose, alginate, chitosan, heparin, and/or heparan sulfate, combinations thereof, and the like.

In aspects, the isotonic hydrogel may include one or more monohydric and/or polyhydric alcohols. For example, the aqueous solution and/or the dried components may include ethanol, 1-propanol, propylene glycol, ethylene glycol, xylitol, sorbitol, erythritol, mannitol, isomalt, maltitol, lactitol, arabitol, ribitol, glycerol, threitol, dulcitol, inositol, iditiol, adonitol, and/or galactitol, combinations thereof, and the like.

In aspects, the isotonic hydrogel may include one or more amino acids. For example, the aqueous solution and/or the dried components may include glutamic acid, aspartic acid, cysteine, tyrosine, taurine, histidine, arginine, and/or lysine, combinations thereof and the like. In some aspects, the aqueous solution and/or the dried components may include polyamino acids of the aforementioned amino acids, such as, but not limited to, polylysine, polyarginine, polyglutamate, polyhistidine, combinations thereof, and the like. In some aspects, the aqueous solution and/or the dried components may include peptides and/or polypeptides rich in the aforementioned amino acids.

In aspects, the isotonic hydrogel may include one or more other biocompatible compounds. For example, the aqueous solution and/or the dried components may include one or more buffers, such as tris(hydroxymethyl)aminomethane (TRIS), bis-tris, tris-hydrochloride, N-2-hydroxyethylpiperazine-N-2-ethane sulfonic acid (HEPES), 3-(N-morpholino) propanesulfonic acid (MOPS), 2-(N-morpholino) ethanesulfonic acid (MES), bicine, N-(2-hydroxyethyl) piperazine (HEPPSO), tris(hydroxymethyl)methylamino]propanesulfonic acid (TAPS), and/or piperazine-N,N′-bis(2-ethanesulfonic acid) (PIPES), combinations thereof, and the like; one or more water-soluble polymers, such as polyethylene glycol, polyvinylpyrrolidone, polyethyleneimine, polyacrylic acid, polyvinyl alcohol, sodium hyaluronate, and the like; one or more quaternary ammonium compounds, such as choline chloride, trimethylamine N-oxide, proline betaine, dimethylsulfoniopropionate, and/or sarcosine betaine, combinations thereof, and the like; and/or one or more surfactants, such as polysorbate 80, polysorbate 20, cetyltrimethylammonium bromide, sodium dodecyl sulfate, poloxamers, triton X-100, sodium deoxycholate, and/or 3-[(3-cholamidopropyl)dimethylammonio]-1-propanesulfonate (CHAPS), combinations thereof, and the like. It will be appreciated that the one or more biocompatible compounds may be provided at any concentration that is sufficient to generate the desired osmotic pressure.

In some aspects, the isotonic hydrogel may include one or more salts in combination with one or more organic acids and/or acidic salts thereof. In some aspects, the isotonic hydrogel may include one or more salts in combination with one or more carbohydrates. In some aspects, the isotonic hydrogel may include one or more salts in combination with one or more organic acids and/or acidic salts thereof and/or one or more carbohydrates. In some aspects, the isotonic hydrogel may include one or more salts in combination with one or more monohydric and/or polyhydric alcohols. In some aspects, the isotonic hydrogel may include one or more salts in combination with one or more amino acids and/or peptides and/or proteins. In some aspects, the isotonic hydrogel may include one or more salts in combination with one or more other biocompatible compounds. In some aspects, the isotonic hydrogel may include one or more salts in combination with one or more organic acids and/or acidic salts thereof, one or more carbohydrates, and/or one or more monohydric and/or polyhydric alcohols. In some aspects, the isotonic hydrogel may include one or more salts in combination with one or more organic acids and/or acidic salts thereof, one or more carbohydrates, one or more monohydric and/or polyhydric alcohols, and/or one or more amino acids and/or peptides and/or proteins. In some aspects, the isotonic hydrogel may include one or more salts in combination with one or more organic acids and/or acidic salts thereof, one or more carbohydrates, one or more monohydric and/or polyhydric alcohols, one or more amino acids and/or one or more other biocompatible compounds.

### Osmotically-Imbalanced Layered Hydrogels

As set forth in the methods for preparing the hydrogels and the osmotic hydrogels, osmotically-imbalanced layered hydrogels can be provided having one or more osmotically-imbalanced layers. It is a further aspect of the present disclosure that the hydrogel can include a plurality of layers such that the layered hydrogel includes one or more protective layers and one or more therapeutic layers.

In some aspects, the osmotically-imbalanced hydrogel may be a layered hydrogel, described in greater detail herein, having one or more osmotically-imbalanced layers. For example, the layered hydrogel may have 2, 3, 4, 5, 6, 7, 8, 9, 10, or more layers. Each layer of the plurality of layers can be independently formulated to be hypertonic, hypotonic, or isotonic.

In aspects, the protective layer(s) of the osmotically-imbalanced hydrogel include isotonic hydrogels such that the hydrogel is osmotically balanced with respect to the adjacent tissue. In aspects, the protective layer of the layered hydrogel is delivered adjacent to the non-targeted tissue.

In aspects, the therapeutic layer(s) of the osmotically-imbalanced hydrogel include osmotically-imbalanced hydrogels such that the hydrogel is hypertonic and/or hypotonic with respect to the adjacent tissue. In aspects, the therapeutic layer of the layered hydrogel is delivered adjacent to the targeted tissue.

In aspects, the therapeutic layer is hypertonic with respect to the targeted tissue. In aspects, the therapeutic layer creates an osmotic gradient, causing hyperosmotic stress of the targeted tissue, leading to cell death. In aspects, the targeted tissue is a cancerous tumor. In aspects, the targeted tissue is a prostate tumor.

In aspects, the therapeutic layer is hypotonic with respect to the targeted tissue. In aspects, the therapeutic layer creates an osmotic gradient, causing hypoosmotic stress of the targeted tissue, leading to cell lysis. In aspects, the targeted tissue is a cancerous tumor. In aspects, the targeted tissue is a prostate tumor.

### Loaded Hydrogels

In aspects, the hydrogel may contain one or more additional materials, such as additional materials to be released as the hydrogel degrades. Such may include embedded materials within the hydrogels as described herein. In some aspects, the hydrogels may be embedded with materials to quickly release material after implantation of the hydrogel. In some aspects, the hydrogels may be embedded with materials to deliver a payload of released material. In some aspects, the hydrogels may be embedded with permanent materials, such as markers, to allow for the rapid identification of the site treated, both during the lifespan of the gel and after the hydrogel has degraded completely.

As set forth in the methods for preparing the hydrogels, two separate solutions are needed, with each solution being prepared with a step of hydrating a dried monomeric unit and a dried complexing molecule. It is a further aspect of the present disclosure that the hydrogel may include one or more additional components therein. It will be apparent that as each of the two solutions needed to form the hydrogel requires hydration of the monomer and the complexing molecule, additional components can be provided either in a dry form and added with the monomeric unit and/or complexing molecule and/or provided in an aqueous form in the hydrating aqueous solution used to hydrate the monomeric unit and/or complexing molecule.

### Embedded Components

In some aspects, the hydrogels of the present disclosure may include one or more embedded components therein. As described herein, the hydrogels of the present disclosure are formed in situ through the application of two separate aqueous solutions that mix together at a desired site. As described herein, one solution includes the monomeric unit and the other solution includes the complexing molecule. It is an aspect of the present disclosure that either of these two solutions may include one or more additional materials therein that become embedded in the hydrogel as it forms. For example, an embedded component may include a tissue marker, a therapeutic agent, a micro and/or nanoparticle, a radiopaque marker, a crystalline therapeutic, a radioactive particle, a radio-protective agent, a radio-sensitizing agent, a reactive oxygen species, or combinations thereof. In some aspects, both solutions may include the same component(s) to be embedded.

In certain aspects of the present disclosure, the embedded component is provided within the hydrogel to deliver a payload of an embedded component, such that a significant portion of the embedded component will be delivered in situ to the patient in a burst release. As identified herein, the burst release can be adjusted or tuned to a particular need, such as by triggering the release with chemical, electrical or mechanical stimulation. In some aspects, the burst release can be adjusted or tuned by varying the concentration of the embedded component that is adsorbed or bound to the surface of the hydrogel.

In certain aspects of the present disclosure, the embedded component is provided within the hydrogel to deliver a payload of an embedded component, such that the entire concentration of the embedded component will be delivered in situ to the patient as it is released from the hydrogel through the degradation thereof over time. As identified herein, the time of degradation of the hydrogel can be adjust or tuned to a particular need. As such, the concentration of the embedded component can similarly be adjust to reflect the degradation of the hydrogel or the corresponding release rate from the hydrogel.

Using PEI and PEG-SCM-2 (or NHS-PEG-SCM-2) as examples of the monomeric unit and the complexing molecule, two solutions are formed that keep these components separate until the time of implantation. For example, the two solutions are prepared by reconstituting dried massed amounts of each in the barrel of a syringe. In some aspects, a double-barreled syringe may be used. In some aspects, the two barrels use a needle that is configured to allow the two solutions to mix as they reach or near the needle end, such that the two solutions mix at or near the point of application. In some aspects, an embedded component is also massed and included with the PEG-SCM-2 and/or PEI prior to reconstitution thereof. Such may allow the embedded component to dissolve or become suspended within the solution.

As set forth herein, the hydrogel may have a set degradation time. It will be appreciated that the degradation time may vary for certain aspects of the degradation may be down to the particular physiology of the patient receiving the hydrogel. The hydrogel will, however, completely degrade eventually. Embedding one or more additional components therein accordingly provides an opportunity to deliver over the lifetime of the hydrogel a payload of embedded content therein. Whether dissolved or suspended, embedded content can be released into the subject as the hydrogel degrades.

In some aspects, the embedded component includes one or more therapeutic agents. The therapeutic can be dissolved within one or both solutions, such as with hydrophilic therapeutic agents. The therapeutic may similarly be prepared as an amorphous or crystalline particle suspension within one or both solutions, such as with hydrophobic compounds. The amount of therapeutic agent(s) embedded within the hydrogel can depend on the desired release rate, taking into consideration the hydrogel degradation rate. For example, loading for a final concentration of 5 μg/mL of hydrogel into a hydrogel that degrades at a rate of 1 mL/day will allow for 5 μg of the therapeutic to be released each day in situ. If the hydrogel occupies 25 mL of space, the hydrogel will provide 125 μg to the patient over the lifetime of the hydrogel. It will also be apparent that this assumes a constant rate of degradation, yet degradation rate need not be constant. In some aspect, the rate of degradation may be affected by the surface are of the hydrogel exposed to the subject's physiology.

In some aspects, the total dose of therapeutic needed may be less as the delivery can be at or near a desired site for treatment. In some aspects, the site for treatment is adjacent or touching the hydrogel. Site-specific delivery may allow for higher efficacy and accordingly lower doses. In some aspects, site-specific delivery can avoid or delay events, such as drug metabolism. In certain aspects, a therapeutic may require metabolism for efficacy. In such aspects, it may be advantageous to use a modified therapeutic to present certain metabolic processes such as hydroxylation, glutathione conjugation, methylation, acetylation, glucuronidation, glycine conjugation, or similar.

In some aspects, the embedded therapeutic is a chemotherapeutic agent. The choice of chemotherapeutic agent(s) may be determined by the type of tumor, malignancy, or cancer. For example, as set forth in the examples, the hydrogel can be utilized to fill between the prostate and the bowel in male subjects undergoing treatment for prostate cancer. Including one or more prostate cancer therapeutic agents therein can provide for site-specific drug delivery of chemotherapeutic(s). For example, the hydrogel may be embedded with docetaxel, degarelix, abiraterone, apalutamide, bicalutamide, cabazitaxel, darolutamide, leuprolide, enzalutamide, flutamide, goserelin, lutetium Lu¹⁷⁷ vipivotide, tetraxetan, olaparib, mitoxantrone, nilutamide, relugolix, sipuleucel-T, radium 223, rucaparib camsylate, or combinations thereof.

Similarly, hydrogels may be utilized to fill resected tissue space, such as in breast tissue. In such aspects, the hydrogel may be embedded with raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

Other choices of therapeutic agent may be embedded. For example, the therapeutic may be chosen from paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, nintedanib, avapritinib, abemaciclib, erdafitinib, fedratinib, nilotinib, nintendanib, palbociclib, pemigatinib, xanthines, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl) adenine), (2-[(3,4-dimethoxyphenyl)methyl]-7-[(1R)-1-hydroxyethyl]-4-phenylbutyl]-5-methyl-imidazo[5,1-f][1,2,4]triazin-4 (1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, theophylline, resveratrol, quercetin, curcumin, chrysin, myricetin, luteolin, apigenin, anthrocyanin, genistein, epigallocatechin gallate, fisetin, astxanthin, tetrahydrocurcumin, imatinib, nintedanib, sorafenib, sunitinib, pazopanib, ROCK inhibitor (Y27632), YAP/TAZ inhibitor (CA3 and verteporfin), YAP/TAZ-TEAD interaction inhibitor (verteporfin, VGLL4 peptide), SRC inhibitor (dasatinib), and combinations thereof.

In some aspects, the therapeutic is a compound or molecule that renders cells sensitive to radiation, such that when the cells receive a dose of radiation, they are more susceptible to respond to the treatment. In some aspect, the hydrogel is embedded with one or more radiosensitizing agents that due to the site-specific delivery render cells in the vicinity of the hydrogel radiosensitive. Examples of such agents may include gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, zinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

In aspects, the embedded component is a compound or molecule that renders cells sensitive to radiation by creating an oxygen rich environments, such that when the targeted tissue receives a dose of radiation, the oxygen creates organic peroxide free radicals targeted tissue is more susceptible to respond to the treatment, while the non-targeted tissue is not affected. In some aspects, the hydrogel includes one or more reactive oxygen species that, due to the site-specific delivery, render the targeted tissue in the vicinity of the hydrogel more sensitive to radiation. In some aspects, the non-targeted tissue does not experience negative effects from the reactive oxygen species. Examples of suitable reactive oxygen species include, but are not limited to peroxides, superoxides, hydroxyl radicals, singlet oxygen, and/or alpha-oxygen, and combinations thereof.

In aspects, the hydrogel may include one or more peroxides. Suitable peroxides include, but are not limited to, peroxy acids, metal peroxides, main group peroxides, and/or organic peroxides, and the like. For example, the aqueous solution and/or the dried components may include formamide peroxide, dicumyl peroxide, dibenzoyl peroxide, diacyl peroxide, Caro's acid, peroxynitric acid, peroxydisulfates, hydrogen peroxide, peroxynitrite, alkyl peroxide, tert-butyl peroxide, combinations thereof, and the like.

In some aspects, the presence of gas nanoparticles or gas nanobubbles within the hydrogel may render adjacent tissue sensitive to radiation treatment. In some aspects, nanobubbles may include a lipid monolayer or bilayer membrane encasing a center of a gas, such as oxygen or perfluorocarbons. In some aspects, the nanobubbles may be released and congregate around and in diseased tissue due to increased permeability. Application of energy, such as with ultrasound can then cause ultracavitation and ultimately shear stress in tumor cells.

In some aspects, the therapeutic agent(s) is a compound or molecule that provides protection to tissue from radiation or is a radioprotective agent. In some aspects, the hydrogels of the present disclosure can be formed or placed in a tissue or in contact with a tissue that is susceptible to exposure to radiation treatment of a neighboring or proximal tissue. The inclusion of a radioprotective agent within the hydrogel offers a site specific delivery to administer radioprotection to tissues that may be sensitive to radiation and thereby reduce the potential for negative impact to normal or healthy tissue. The radioprotective agent(s) may be selected or chosen from amifostine, palifermin, super oxide dismutase, tetracycline, genistein, captopril, Lisinopril, 3,3′-diindolylmethane, rapamycin, CBLB502, ON01210, γ-tocotrienol, δ-tocotrienol, R-spondin 1, transforming growth factor β3, mesenchymal stem cells, bone marrow stromal cells, myeloid progenitor cells, antioxidants, or combinations thereof.

In aspects, the embedded component is a compound or molecule that provides protection to tissue from radiation or is a radioprotective agent. In some aspects, the hydrogels of the present disclosure can be formed or placed in a tissue or in contact with a tissue that is susceptible to exposure to radiation treatment of a neighboring or proximal tissue. The inclusion of a radioprotective agent within the hydrogel offers a site-specific delivery to administer radioprotection to tissues that may be sensitive to radiation and thereby reduce the potential for negative impact to normal or healthy tissue. In aspects, the radioprotective agent is an antioxidant. Examples of suitable antioxidants include, but are not limited to, butylated hydroxytoluene, N-acetyl-DL-tryptophan, butylated hydroxytoluene, propyl gallate d-alpha tocopheryl, sodium metabisulfite, ascorbic acid, vitamin E, polyphenols, carotenoids, coenzyme Q10, combinations thereof, and the like.

As described above, the hydrogels of the present disclosure may be layered, such as two different hydrogels or two similar hydrogels stacked together. It will be apparent that different layers of the hydrogel may have different embedded materials therein. For example, in the scenario where a hydrogel is placed between the prostate and the bowels/rectum of a subject, radiosensitizing agents may be embedded within a hydrogel layer in contacted with the treated tissue, while radioprotective agents may be embedded in layer(s) touching non-treated or healthy tissue.

### Example 1

In some aspects, one or both solutions include a suspension of one or more materials therein, such that the materials become embedded throughout the hydrogel as it forms. Such materials are typically not soluble or only partially soluble in the aqueous solutions. It is an aspect that the diameter or width at the largest cross sectional view of each particle be sufficiently small to avoid blocking any delivery means, such as the needle of a syringe. It will also be apparent that the density of each particle be maintained such that not every particle will slide through the mixed liquids before the hydrogel is sufficiently formed to suspend them therein.

In some aspects of the present disclosure, the particles have a diameter of about 300 μm or less, including about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 2309, 240, 250, 260, 270, 280, 290, and 300 μm. In some aspects, the particles are of about 0.1 μm to about 10 μm, about 0.1 μm to about 100 μm, about 0.1 to about 300 μm, about 0.5 μm to about 1 μm, about 0.5 μm to about 5 μm, about 0.5 μm to about 10 μm, about 0.5 μm to about 50 μm, about 0.5 μm to about 100 μm, about 0.5 μm to about 200 μm, about 0.5 μm to about 300 μm, about 1 μm to about 5 μm, about 1 μm to about 10 μm, about 1 μm to about 50 μm, about 1 μm to about 100 μm, about 1 μm to about 200 μm, about 1 μm to about 300 μm, 5 μm to about 10 μm, about 5 μm to about 50 μm, about 5 μm to about 100 μm, about 5 μm to about 200 μm, about 5 μm to about 300 μm, about 10 μm to about 50 μm, about 10 μm to about 100 μm, about 10 μm to about 200 μm, about 10 μm to about 300 μm, about 50 μm to about 100 μm, about 50 μm to about 200 μm, about 50 μm to about 300 μm, about 100 μm to about 200 μm, about 100 μm to about 300 μm, or about 200 μm to about 300 μm.

In some aspects, the particles may be considered microspheres or microparticles or nanoparticles. It is to be understood that reference to such still includes all aspects of “particles” as used herein.

In some aspects, the particles are crystalline therapeutic particles and/or amorphous therapeutic particles. In other aspects, the particles are a biodegradable polymer with one or more therapeutics and/or radiopaque markers embedded therein. In other aspects, the particles are a biocompatible two- or three-phase microparticles that includes a radioactive isotope or a compound including at least one radioactive element.

### Biodegradable Polymer Microparticles

In aspects of the present disclosure, the hydrogels may be embedded with microparticles or have microparticles suspended therein. In some aspects, the microparticles are of a biodegradable polymer.

In some aspects, one or more therapeutic agent(s) may be embedded within a biopolymer particle or microparticle. In some aspects, one or more radiopaque markers may be embedded within a biopolymer particle or microparticle. In some aspects, both a therapeutic agent and a radiopaque marker may be embedded within a microparticle. All of such may be achieved by dissolving a therapeutic in a solution with a polymer dissolved therein and the liquid evaporated, such as emulsion evaporation.

The therapeutic agent candidates of the microparticles are discussed herein. As may be needed the hydrophobicity can be adjusted for solubilization or a more hydrophilic biopolymer selected to allow for the microparticle to form. It will be appreciated that the therapeutic and/or radiopaque marker need to be soluble in the solvent. With hydrophilic molecules, such can be achieved through chemical modification.

The radiopaque markers of the present disclosure may include methylated triiodobenzoic acid (TIBA-Me). The radiopaque marker TIBA, or triiodo benzoic acid (or 2,3,5 triiodobenzoic acid) can be methylated to for TIBA-Me or methyl-2,3,5,triiodobenzoate. For example, as set forth in the examples, reacting TIBA with n,n′-dicyclohexylcarbodiimide (DCC) and methanol allows a methyl group to replace the hydroxyl in the appended carboxylic acid. It will be appreciated that the methylation of TIBA increases the hydrophobicity thereof.

In some aspects, the present disclosure concerns radiopaque markers and/or therapeutic agent(s) embedded in a biodegradable polymer microparticle, which itself is embedded in the hydrogels of the present disclosure. Degradation of the hydrogel allows for the sustained release of the biodegradable polymer microparticles from the hydrogel. The degradation of the biodegradable polymer thereof allows for the further sustained release of the radiopaque marker and/or therapeutic agent(s) therein.

Biodegradable polymer microparticles may be prepared the evaporation of a solvent with a bioabsorbable/biodegradable polymer and at least one therapeutic and/or radiopaque agent therein. In some aspects, the solvent is of dichloromethane (DCM) or ethyl acetate (EtOAc). Polymers may include a network of a poly-glycolic acid (PGA), poly-lactic acid (PLA) and a poly-L-lactic acid (PLLA). Other bioabsorbable polymers that can be utilized in combination or alone for the microparticles include polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), and co-polymers with various ratios thereof. In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, the polymer microparticle is also loaded or embedded with an antioxidant, such as BHT.

In some aspects, the hydrogel of the present disclosure may function as a spacer to separate and/or support one or more tissues or organs within a cavity of a subject. In some aspects, the present disclosure concerns a radiopaque hydrogel spacer using encapsulated biodegradable polymer microparticles. The encapsulation of the marker within the polymer microparticles retains the radiopaque marker (e.g. iodine) in the hydrogel without using covalent bonds to link the marker in the hydrogel network. Instead, the marker is encapsulated in a biodegradable polymer microparticles, such as PLGA, and physically trapped inside the hydrogel network. The biodegradation profile of the hydrogel system, its dimensional stability and the radiopaque molecule retention can be designed to provide desired use life suitable for patients with prostate cancer, pancreatic cancer and other cancer types that will benefit with a spacer being created to protect the adjacent healthy tissue or organs during radiation therapy. Such may also be used in other applications where the visibility of the hydrogel is important during or after the gel delivery to insure or confirm the hydrogel is applied on target site with precision.

In some aspects, the concentration density of the therapeutic agent in microparticle may be from 0.1 μg/mm² to 10 μg/mm², from 0.1 μg/mm² to 8 μg/mm², from 0.1 μg/mm² to 6 μg/mm², from 0.1 μg/mm² to 4 μg/mm², from 0.1 μg/mm² to 2 μg/mm², from 0.1 μg/mm² to 1 μg/mm², from 1 μg/mm² to 10 μg/mm², from 1 μg/mm² to 8 μg/mm², from 1 μg/mm² to 6 μg/mm², from 1 μg/mm² to 4 μg/mm², from 1 μg/mm² to 2 μg/mm², from 2 μg/mm² to 10 μg/mm², from 2 μg/mm² to 8 μg/mm², from 2 μg/mm² to 6 μg/mm², from 2 μg/mm² to 4 μg/mm², from 4 μg/mm² to 10 μg/mm², from 4 μg/mm² to 8 μg/mm², from 4 μg/mm² to 6 μg/mm², from 6 μg/mm² to 10 μg/mm², from 6 μg/mm² to 8 μg/mm², or from 8 μg/mm² to 10 μg/mm².

### Radioactive Microparticles

In aspects of the present disclosure, the hydrogels may be embedded with radioactive microspheres or have radioactive microparticles suspended therein to deliver therapeutic radiation at a target site. In some aspects, the microparticles include a radioactive isotope or a compound including at least one radioactive element. The radioactive isotope or compound may include a radioisotope such as a beta-gamma emitter that or a gamma emitter that emits sufficient gamma radiation to enable imaging. Examples of specific radioactive isotopes include, without limitation, bismuth-213, boron-10, cesium-131, cesium-137, cobalt-60, dysprosium-165, erbium-169, holmium-166, iodine-125, iodine-131, iridium-192, iron-59, lead-212, lutetium-177, molybdenum-99, palladium-103, phosphorus-32, potassium-42, radium-223, rhenium-186, rhenium-188, samarium-153, selenium-75, sodium-24, strontium-89, technetium-99m, thorium-227, xenon-133, ytterbium-169, ytterbium-177, and yttrium-90. Some other examples include actinium-225, astatine-211, bismuth-213, carbon-11, nitrogen-13, oxygen-15, fluorine-18, cobalt-57, copper-64, copper-67, fluorine-18, gallium-67, gallium-68, germanium-68, indium-111, iodine-123, iodine-124, krypton-81m, rubidium-82, strontium-82, and thallium-201. The microparticles may include a compound including any of the foregoing radioactive isotopes. In some aspects, the microparticles may include yttrium-90 or a compound including a yttrium-90 atom such as yttrium phosphate (⁹⁰YPO₄), yttrium sulfate (⁹⁰Y₂ (SO₄)₃) or (⁸⁹Y₉₀Y(SO₄)₃), or yttrium carbonate (⁹⁰Y₂ (CO₃)₃) or (⁸⁹Y₉₀Y (CO₃)₃).

The two- or three-phase microparticles may include a cured first biocompatible resin with a radioisotope or compound thereof therein that is internal to a second and/or third cured resin. The second and third resins may be of the same materials as the first. The two or three phase microparticles may further include one or more therapeutic agents as set forth herein. In some aspects, the radioactive microparticles are as set forth in WO 2019/222700, which is hereby incorporated by reference in its entirety.

### Tissue Marker Microparticles

In aspects of the present disclosure, the hydrogels may be embedded with a permanent or semi-permanent material that is detectable so as to inform of the location thereof in situ within a subject. While the presence of the hydrogel itself need not be permanent, it is of value to the subject for a physician or health care provider to be able to determine the former site that the hydrogel occupied. While radiopaque markers as described herein can locate the hydrogel, additional materials may be embedded to demark the site on a more permanent basis. As such, embedding materials such as titanium particles or nitinol particles or PVA particles in the hydrogel provide materials that can be detected over a prolonged period of time. In some aspects, it may be of interest to insert a tissue marker as the gel forms in situ in order to increase the surface area available and enhance the ability of a user to detect and locate the tissue marker.

### Kits and Methods of Use

In aspects, the present disclosure also concerns kits to provide the hydrogels or allow a user to prepare the hydrogels as set forth herein. In some aspects, the kit may include a first aqueous solution and a dried monomeric unit. As described herein, it is of benefit to reconstitute the monomeric unit prior to forming the hydrogel. Similarly, a second aqueous solution can be provided to reconstitute dried complexing molecule. The first and second aqueous solutions may be the same or may be different. As such, the kit may include one or two containers for reconstituting the complex molecule and the monomeric unit.

As also described herein, the hydrogels may include additional features, such as embedded components. In some aspects, the kits may include such as well.

As also described herein, in some aspects, the hydrogel includes varying osmotic properties. In some aspects, the kits may include the required salts or similar components to allow a user to prepare the hydrogel properly.

In some aspects, the kits may include means to mix the two solutions. As described herein, the hydrogels of the present disclosure may form rapidly. As such, mixing may be performed at or near the cavity to be filled with the hydrogel. In some aspects, such may include two separate syringe barrels operably connected to a single needle point. Dispensing both solutions from the barrels toward the single or shared needle point can allow the two to contact each other as the fluid is dispensed from the needle point and into the cavity.

It will also be understood that kits may optionally include appropriate packing and/or directions or links thereto that allow a user to prepare the hydrogel.

In some aspects, the present disclosure concerns methods of using the hydrogels as set forth herein. Such may include contact the monomeric unit solution with the complexing molecule solution at/in/above a cavity within a subject and have the hydrogel form therein.

As described herein, the formation of the hydrogel within the cavity allows for the hydrogel to provide structural support and or forced spacing between tissues/organs surrounding the cavity. As also described herein, the hydrogel may include one or more particular additional features, such as an osmotic imbalance, a therapeutic, a microparticle, a radiosensitizing agent, a radiopaque marker, a radioprotective agent, and so on. The additional inclusion of such within the hydrogel offers additional aspects for treating or aiding the subject in addition to the benefits provided by filling the cavity space.

As also described herein, the hydrogels of the present disclosure offer a variety of degradation times. Accordingly the methods of use also include providing the hydrogel to the cavity for particular amounts of time, as well as not having to remove or excise the hydrogel when its presence is no longer needed.

### EXAMPLES

The two-solution hydrogel chemistry requires the two components to be packaged separately before use. It is worth considering that with respect to long-term storage, some materials are sensitive to moisture and the dry and wet components accordingly kept apart or isolated in separate packaging. Reconstitution of the dry components including PEG, PEI, rHSA and the PLGA particles is necessary prior to preparing the hydrogel. The hydrogel preparation at a clinical setting starts with hydration of the two components. Each component can be hydrated individually by connecting a first syringe with a wet component and a second syringe with dry components and pushing the wet component pack and forth through the connector. A parallel design with dual chamber syringes can be used to hydrate component one and component two at the same time through a connector using similar manual process. Alternatively, other hydration techniques can be used as well. For example, a magnetic disc or bar can be preloaded in the syringe and once the wet component is introduced and capped, it can be hydrated by position the syringes on the magnetic stirring base and allow the magnetic disc or bar to stir and mix or hydrate. Alternatively, hydration process can be achieved by placing the syringes in an ultrasonic and/or mechanical vibrating system such as a vortex mixer. A fixture can be used to hold the syringes in place during vortex mixing/hydration.

After the two components are fully hydrated and PLGA particle are dispersed, the mixing and delivery of the hydrogel follows. The mixing process is critical due to the system's fast crosslinking speed (short gel time). Premature mixing before delivery could result in clogged needle while not enough mixing could lead to failed integrity of the gel structure and shape during its service life.

The mixing of the two components can be achieved during the fast flow through a long needle and further mixing by turbulence created after exiting the delivery needle tip (picture in the word document in the original IDR filing). The mixing can be also assisted with BD Progel delivery system where the mixing is achieved by spray at the delivery tip. Alternatively, the mixing can be further assisted with a torturous path mixing section which can be positioned immediately at the end of the Y-joint. The delivery needle can be designed to its proper length suited for targeted application.

Once the mixing and delivery process starts, it needs to proceed continuously until completion. The delivery process cannot be stopped or paused because the quick reaction gel will clog the system.

Hydrogels were prepared with PEI and PEG as variables for the linker and the complexing molecule, as well as the presence of a core or a central PEG for 2 arms only. Table 1 sets forth the MW of the PEG and PEI, the presumed number of amines for cross-linking, the time for gel formation and the time for gel degradation.

TABLE 1

| | | | | PEG | | | PEI | | | | | |
|---|---|---|---|---|---|---|---|---|---|---|---|---|
| | | PEG | NHS: | mass | PEG | PEI | mass | Gel | | Degradation | | # |
| ARMS | CORE | MW | amine | (g) | pH | MW | (g) | Time | | time | | amines |
| 8 | Tripentaerythritol | 15000 | 2.21 | 0.4815 | N/A | 2k | 0.0130 | <1 | s | 2 | days | 18 |
| 4 | Tripentaerythritol | 40000 | 0.22 | 0.2600 | N/A | 2k | 0.0130 | 5 | s | Hours | | 18 |
| 2 | N/A | 2000 | 2.21 | 0.1300 | N/A | 2k | 0.0130 | 30 | s | N/A | | 18 |
| 2/4 | N/A/ | 2000/40000 | 2.21 | 0.2554/0.0505 | N/A | 2k | 0.0133 | 13 | s | N/A | | 18 |
| | Tripentaerythritol | | | | | | | | | | | |
| 2 | N/A | 2000 | 1.11 | 0.1300 | N/A | 2k | 0.0130 | Not | | >4 | days | 18 |
| | | | | | | | | recorded | | | | |
| 2 | N/A | 2000 | 0.05 | 0.1300 | N/A | 2k | 0.5400 | Not | | >4 | days | 18 |
| | | | | | | | | recorded | | | | |
| 2 | N/A | 2000 | 0.05 | 0.1300 | N/A | 2k | 0.5400 | Not | | >4 | days | 18 |
| | | | | | | | | recorded | | | | |
| 2 | N/A | 2000 | 0.21 | 0.1300 | N/A | 2k | 0.1350 | Not | | >4 | days | 18 |
| | | | | | | | | recorded | | | | |
| 2 | N/A | 2000 | 0.11 | 0.1300 | N/A | 2k | 0.2700 | Not | | >4 | days | 18 |
| | | | | | | | | recorded | | | | |
| 2 | N/A | 2000 | 1.11 | 0.1298 | N/A | 2k | 0.0133 | Not | | N/A | | 18 |
| | | | | | | | | recorded | | | | |
| 2/8 | N/A/ | 2000/15000 | 2.21 | 0.2076/0.1023 | N/A | 2k | 0.0134 | 1 | s | N/A | | 18 |
| | Tripentaerythritol | | | | | | | | | | | |
| 2 | N/A | 2000 | 1.11 | 0.1316 | N/A | 2k | 0.0136 | Not | | N/A | | 18 |
| | | | | | | | | recorded | | | | |
| 2 | N/A | 3000 | 2.21 | 0.3886 | N/A | 2k | 0.0131 | Not | | N/A | | 18 |
| | | | | | | | | recorded | | | | |
| 2 | N/A | 5000 | 2.21 | 0.6445 | 3.86 | 2k | 0.0131 | <1 | s | Hours | | 18 |
| 2 | N/A | 5000 | 2.21 | 0.4011 | 3.9 | 2k | 0.0081 | 3-5 | s | Hours | | 18 |
| 2 | N/A | 5000 | 2.21 | 0.2959 | 3.99 | 2k | 0.0058 | >30 | s | Hours | | 18 |
| 2 | N/A | 3400 | 2.21 | 0.3993 | 5.96 | 2k | 0.0123 | 1 | s | Hours | | 18 |
| 2 | N/A | 3400 | 2.21 | 0.3004 | 6 | 2k | 0.0091 | 15-30 | s | Hours | | 18 |
| 8 | Tripentaerythritol | 10000 | 2.21 | 0.1700 | 4.21 | 2k | 0.0068 | 2-3 | s | N/A | | 18 |
| 4 | Tripentaerythritol | 10000 | 2.21 | 0.3996 | 3.65 | 2k | 0.0079 | <1 | s | N/A | | 18 |
| 4 | Tripentaerythritol | 10000 | 2.21 | 0.2007 | 3.61 | 2k | 0.0040 | 40 | s | N/A | | 18 |
| 2 | N/A | 5000 | 2.21 | 0.3999 | N/A | 2k | 0.0087 | 3 | s | Hours | | 18 |
| 2 | N/A | 5000 | 1.75 | 0.3993 | N/A | 2k | 0.0103 | 2 | s | Hours | | 18 |
| 2 | N/A | 5000 | 1.00 | 0.3996 | N/A | 2k | 0.0182 | <1 | s | Hours | | 18 |
| 2 | N/A | 5000 | 1.75 | 0.4005 | N/A | 2k | 0.0101 | 5-10 | s | <20 | hours | 18 |
| 2 | N/A | 5000 | 1.00 | 0.3992 | N/A | 2k | 0.0180 | Immediate | | <20 | hours | 18 |
| 2 | N/A | 5000 | 0.50 | 0.4006 | N/A | 2k | 0.0357 | Immediate | | 2 | hours | 18 |
| 2 | N/A | 3000 | 1.75 | 0.3104 | N/A | 2k | 0.0132 | 2-3 | s | | | 18 |
| 2 | N/A | 2000 | 1.75 | 0.2000 | N/A | 2k | 0.0125 | 5-10 | s | | | 18 |
| 2 | N/A | 2000 | 1.00 | 0.2008 | N/A | 2k | 0.0223 | Immediate | | | | 18 |
| 2 | N/A | 2000 | 2.00 | 0.2008 | N/A | 25k | 0.0120 | 3-5 | s | | | 192 |
| 2 | N/A | 2000 | 2.00 | 0.1009 | N/A | 25k | 0.0057 | No gel | | | | 192 |
| 2 | N/A | 2000 | 3.00 | 0.1994 | N/A | 25k | 0.0078 | No gel | | | | 192 |
| 2 | N/A | 2000 | 2.50 | 0.2008 | N/A | 25k | 0.0091 | 5-10 | m | | | 192 |
| 2 | N/A | 3510 | 1.75 | 0.3308 | N/A | 2k | 0.0118 | 8-10 | s | <24 | hours | 18 |
| 2 | N/A | 3554 | 2.00 | 0.3304 | 2.48 | 2k | 0.0113 | 10-15 | s | ~18 | months | 17 |
| 2 | N/A | 3554 | 1.50 | 0.3303 | N/A | 2k | 0.0145 | 2 | s | ~18 | months | 17 |
| 2 | N/A | 3554 | 1.75 | 0.3301 | N/A | 2k | 0.0124 | 2 | s | ~18 | months | 17 |
| 2 | N/A | 3554 | 1.75 | 0.3794 | N/A | 2k | 0.0142 | 2 | s | ~18 | months | 17 |
| 2 | N/A | 3554 | 1.85 | 0.3810 | N/A | 2k | 0.0135 | 3 | s | ~18 | months | 17 |

| 2 | N/A | 3554 | 1.75 | 0.3605 | N/A | 2k | 0.0137 | Immediate | | N/A | 17 |
|---|---|---|---|---|---|---|---|---|---|---|---|
| 2 | N/A | 3554 | 1.40 | 0.2884 | N/A | 2k | 0.0137 | 2 | sl | N/A | 17 |
| 2 | N/A | 3554 | 1.12 | 0.2307 | N/A | 2k | 0.0137 | 8 | s | N/A | 17 |
| 2 | N/A | 5147 | 1.75 | 0.3598 | N/A | 2k | 0.0094 | Immediate | | | 17 |
| 2 | N/A | 3554 | 1.75 | 0.2002 | N/A | 2k | 0.0081 | 1 | s | N/A | 17 |
| 2 | N/A | 3554 | 2.21 | 0.2001 | N/A | 2k | 0.0061 | 3 | s | N/A | 17 |
| 2 | N/A | 3500 | 2.21 | 0.1305 | N/A | 2k | 0.0039 | 3 | s | N/A | 17 |
| 2 | N/A | 3500 | 2.21 | 0.1794 | N/A | 2k | 0.0057 | 2 | s | N/A | 17 |
| 2 | N/A | 3500 | 2.21 | 0.2002 | N/A | 2k | 0.0060 | 2 | s | N/A | 17 |
| 2 | N/A | 5000 | 2.21 | 0.2506 | N/A | 2k | 0.0053 | 2 | s | N/A | 17 |
| 2 | N/A | 3500 | Not | 0.2008 | N/A | N/A | N/A | 2 | s | N/A | 17 |
| | | | calculated | | | | | | | | |
| 2 | N/A | 5000 | Not | 0.2501 | N/A | N/A | N/A | 2 | s | N/A | 17 |
| | | | calculated | | | | | | | | |
| 2 | N/A | 2000 | 2.00 | 0.2007 | N/A | 25k | 0.0124 | No gel | | N/A | 192 |
| 2 | N/A | 2000 | 2.00 | 0.2008 | N/A | 25k | 0.0124 | No gel | | N/A | 192 |
| 2 | N/A | 2000 | 2.00 | 0.2503 | N/A | 25k | 0.0160 | No gel | | N/A | 192 |
| 2 | N/A | 2000 | 2.00 | 0.2256 | N/A | 2k | 0.0131 | 3 | s | N/A | 17 |
| 2 | N/A | 2000 | 2.00 | 0.2244 | N/A | 2k | 0.0137 | 4 | s | N/A | 17 |
| 2 | N/A | 2000 | 2.00 | 0.2254 | N/A | 2k | 0.0135 | 4 | s | N/A | 17 |
| 2 | N/A | 4000 | 2.21 | 0.2506 | N/A | 2k | 0.0068 | 6 | s | N/A | 17 |
| 2 | N/A | 3400 | 2.21 | 0.2490 | N/A | 2k | 0.0078 | 2 | s | N/A | 17 |
| 2 | N/A | 3400 | 2.21 | 0.2250 | N/A | 2k | 0.0069 | 2 | s | N/A | 17 |
| 2 | N/A | 3400 | 2.21 | 0.1297 | N/A | 2k | 0.0040 | 80 | s | N/A | 17 |
| 2 | N/A | 3400 | 2.21 | 0.1810 | N/A | 2k | 0.0058 | 4 | s | N/A | 17 |
| 2 | N/A | 2000 | 2.00 | 0.2248 | N/A | 2k | 0.0130 | 3 | s | N/A | 17 |
| 2 | N/A | 3400 | 2.21 | 0.2252 | N/A | 2k | 0.0072 | 3 | s | N/A | 17 |
| 2 | N/A | 3400 | 2.21 | 0.2253 | N/A | 2k | 0.0069 | 3 | s | N/A | 17 |
| 2 | N/A | 3400 | 2.21 | 0.2253 | N/A | 2k | 0.0071 | 3 | s | N/A | 17 |

### Example 2

PEG-SG hydrogels with differing arms, MW, and amounts of rHA were prepared and assessed for degradation. It was observed that higher MW of SG₈ increased degradation time that SG₈ degraded faster than SG₄, and that higher rHA allowed for slower degradation.

TABLE 2

| PEG Type | rHA Solution | Degradation Time |
|---|---|---|
| PEG-(SG)₈ 10k MW | 10% | 122 days |
| PEG-(SG)₈ 10k MW | 20% | 176 days |
| PEG-(SG)₈ 20k MW | 10% | 166 days |
| PEG-(SG)₈ 20k MW | 20% | 224 days |
| PEG-(SG)₄ 10k MW | 10% | 211 days |

### Example 3

It was endeavored to make a radiopaque hydrogel spacer using encapsulated biodegradable microparticles. This allows the hydrogel to retain the iodine molecule (radiopaque marker) without using covalent bonds to link iodine molecule in the hydrogel network. Instead, the iodine molecule was encapsulated in a biodegradable polymer PLGA microparticles and physically trapped inside the hydrogel network.

2,3,5-triiodobenzoic acid (TIBA) is a common radiopaque molecule which can be used as a radiopaque marker for applications such as implanted spacers. Chemical modification on TIBA molecule is necessary to enable consistent and stable encapsulated microparticles. Several TIBA derivatives have been synthesized and TIBA-Me is selected to be encapsulated into PLGA microparticles for its solubility in particle manufacturing process, its good retention in the particles and low toxicity.

Microparticle encapsulation of TIBA-Me in polylactic glycolic acid copolymer (PLGA) is achieved by creating emulsion of PLGA/TIBA-Me (Oil phase) in PVA solution in water (aqueous phase). The stable emulsion is produced by high-speed homogenization. The emulsion is then allowed to evaporate the solvent in the oil phase by low-speed agitation. After the solvent is evaporated, the particles are collected after centrifugation, washing and drying process. This scheme illustrates the overview of the process. Alternatively, the emulsion can be produced using a micropore membrane process where the oil phase is pushed through a membrane with uniform pores into the aqueous phase while a continuous shear force is applied to create uniform droplets which result in narrowly distributed dry particles/beads following the same evaporation, purification and collection process as described in homogenization process. The particle size can be controlled by selecting different pore size in the membrane. Other parameters such as oil phase solid content, oil phase flow rate and agitation speed can also be used to control the particles size.

Radiopaque hydrogel can be made in situ with a two-component reactive system where component one contains multifunctional polyethylene glycol-SG which is dissolved in water, component two contains albumin, PLGA/TIBA-me particles and water with optional surfactants to control excess foam formation during reconstitution. Small amount of PEG can also be added to component two to help dispersion and the stability of the ingredients. The component one and component two are then introduced and mixed during delivery process to the target site, it then crosslink into a three-dimensional network and forms a flexible but structurally stable radiopaque hydrogel. The radiopaque microparticles do not participate or alter the crosslink reaction, and are physically trapped inside the gel network. The incorporation of the microparticle will not negatively impact the mechanical properties and stability of the hydrogel as compared with chemical coupling approach.

The radiopaque hydrogel is designed to be bioresorbable after its use. Both PEG hydrogel and PLGA particles can be designed to have a matched degradation profile, e.g., up to three month in the situation of a spacer application in prostate cancer radiation therapy. The molecular weight of PEG and PLGA, the lactic acid to glycolic acid ratio in PLGA, the crosslinking density/PEG functional groups are the primary parameters that can be adjusted to achieve the target use life. The following table shows an example of the two-component system radiopaque hydrogel.

Hydrogel properties are studied at 37° C. for extended time. Both hydrogel structure and the radiopaque property can be maintained for its use life supported by the data from modulus measurement and iodine molecule retention/leaching test. The loss of radiopaque molecule is limited to be less than 6% of the original loading.

Three hydrogel samples at different PLGA particle loading containing TIBA-Me iodine molecule were tested. The samples were sandwiched between 1 inch thick pork meat with skin. It is demonstrated the hydrogel is detectable using a clinical CT. At iodine level of 0.5% and 2.5% based on hydrogel weight, both samples showed much brighter appearance while the hydrogel with no PLGA particles appear dark and not very distinguishable from the tissue. Hounsfield Units (HU) values are used to assess the radiopacity and the results showed higher value as iodine content increase in the hydrogel

It is also demonstrated that the gel time (crosslinking reaction time) can be adjusted to meet mixing and delivery needs. The sodium bicarbonate level is effective to adjust the gel time. This is important when the end application needs the optimum gel time for consistent delivery performance and dimensional control. Too short gel time may cause delivery problems such as prematurely clog the delivery needle. While too long gel time may cause inconsistent hydrogel shape and integrity.

For the application in prostate cancer radiation therapy, a target range of the gelation time is about 5 to 10 seconds. It can be optimized considering other factors such as mixing delivery technique used.

Aspects may be further described with respect to the following embodiments:

In a first embodiment, alone or in combination with any other embodiment herein, the present disclosure relates to a hydrogel composition for in situ formation within a cavity, comprising a polymerized complex of at least one monomeric unit, at least one complexing molecule and an aqueous solution, wherein the monomeric unit comprises a terminal structure, a linker and a core and wherein the complexing molecule is chosen from recombinant albumin, polyethyleneimine (PEI), and poly-lysine.

In a second embodiment, alone or in combination with any other embodiment herein, the present disclosure relates to a hydrogel composition wherein the linker comprises polyethylene glycol (PEG).

In a third embodiment, alone or in combination with any other embodiment herein, the present disclosure relates to a hydrogel composition wherein the linker is of sufficient length to provide the hydrogel with a molecular weight (MW) of between 1 kDa and 100 kDa.

In a fourth embodiment, alone or in combination with any other embodiment herein, the present disclosure relates to a hydrogel composition, wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a fifth embodiment, alone or in combination with any other embodiment herein, the present disclosure relates to a hydrogel composition wherein the core is chosen from pentaerythritol, hexaglycerol, tripentaerythritol, or glycerol.

In a sixth embodiment, alone or in combination with any other embodiment herein, the present disclosure relates to a hydrogel composition wherein the monomeric unit has 2, 4, or 8 linker arms extending from the core.

In a seventh embodiment, alone or in combination with any other embodiment herein, the present disclosure relates to a hydrogel composition wherein an ester of the monomeric unit complexes with an amine or imine of the complexing molecule.

In an eighth embodiment, alone or in combination with any other embodiment herein, the present disclosure relates to a hydrogel composition wherein a molar ratio of ester of the monomeric unit to amine or imine of the complexing molecule is of from 0.05 to 3.

In a ninth embodiment, alone or in combination with any other embodiment herein, the present disclosure relates to a hydrogel composition wherein the molar ratio of ester of the monomeric unit to amine or imine of the complexing molecule is of 1.5 to 2.

In a tenth embodiment, alone or in combination with any other embodiment herein, the present disclosure relates to a hydrogel composition wherein the complexing molecule comprises from 2 to 60% weight/volume (w/v) of the hydrogel.

In an eleventh embodiment, alone or in combination with any other embodiment herein, the present disclosure relates to a hydrogel composition wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a twelfth embodiment, alone or in combination with any other embodiment herein, the present disclosure relates to a hydrogel composition wherein the hydrogel has a pH of between 7.5 and 11.0.

In a thirteenth embodiment, alone or in combination with any other embodiment herein, the present disclosure relates to a hydrogel composition wherein the monomeric unit is NHS-PEG-SG-8, NHS-PEG-SG-4, or NHS-PEG-SG-2.

In a fourteenth embodiment, alone or in combination with any other embodiment herein, the present disclosure relates to a hydrogel composition wherein the monomeric unit is NHS-PEG-SS-8, NHS-PEG-SS-4, or NHS-PEG-SS-2.

In a fifteenth embodiment, alone or in combination with any other embodiment herein, the present disclosure relates to a hydrogel composition wherein the monomeric unit is NHS-PEG-SCM-8, NHS-PEG-SCM-4, or NHS-PEG-SCM-2.

In a sixteenth embodiment, alone or in combination with any other embodiment herein, the present disclosure relates to a hydrogel composition wherein the complexing molecule is rHA.

In a seventeenth embodiment, alone or in combination with any other embodiment herein, the present disclosure relates to a hydrogel composition wherein the complexing molecule is PEI.

In an eighteenth embodiment, alone or in combination with any other embodiment herein, the present disclosure relates to a hydrogel composition further comprising an embedded component within the hydrogel.

In a nineteenth embodiment, alone or in combination with any other embodiment herein, the present disclosure relates to a hydrogel composition wherein the embedded component comprises a therapeutic agent.

In a twentieth embodiment, alone or in combination with any other embodiment herein, the present disclosure relates to a hydrogel composition wherein the therapeutic agent is dissolved within the hydrogel.

In a twenty-first embodiment, alone or in combination with any other embodiment herein, the present disclosure relates to a hydrogel composition wherein the therapeutic agent is in a crystalline or amorphous solid form suspended throughout the hydrogel.

In a twenty-second embodiment, alone or in combination with any other embodiment herein, the present disclosure relates to a hydrogel composition wherein the therapeutic agent is a chemotherapeutic agent chosen from docetaxel, degarelix, abiraterone, apalutamide, bicalutamide, cabazitaxel, darolutamide, leuprolide, enzalutamide, flutamide, goserelin, lutetium Lu177 vipivotide, tetraxetan, olaparib, mitoxantrone, nilutamide, relugolix, sipuleucel-T, radium 223, rucaparib camsylate, or combinations thereof.

In a twenty-third embodiment, alone or in combination with any other embodiment herein, the present disclosure relates to a hydrogel composition wherein the therapeutic agent is a chemotherapeutic agent chosen from raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

In a twenty-fourth embodiment, alone or in combination with any other embodiment herein, the present disclosure relates to a hydrogel composition wherein the embedded component is a radiosensitizing agent chosen from gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, xinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

In a twenty-fifth embodiment, alone or in combination with any other embodiment herein, the present disclosure relates to a hydrogel composition wherein the embedded component is a radioprotective agent chosen from amifostine, palifermin, super oxide dismutase, tetracycline, genistein, captopril, Lisinopril, 3,3′-diindolylmethane, rapamycin, CBLB502, ON01210, γ-tocotrienol, δ-tocotrienol, R-spondin 1, transforming growth factor β3, mesenchymal stem cells, bone marrow stromal cells, myeloid progenitor cells, antioxidants, or combinations thereof.

In a twenty-sixth embodiment, alone or in combination with any other embodiment herein, the present disclosure relates to a hydrogel composition wherein the embedded component comprises gas nanobubbles.

In a twenty-seventh embodiment, alone or in combination with any other embodiment herein, the present disclosure relates to a hydrogel composition wherein the embedded component comprises a peroxide.

In a twenty-eighth embodiment, alone or in combination with any other embodiment herein, the present disclosure relates to a hydrogel composition wherein the embedded component comprises a compound that allows for the production of reactive oxygen species from radiotherapy.

In a twenty-ninth embodiment, alone or in combination with any other embodiment herein, the present disclosure relates to a hydrogel composition wherein the embedded component comprises a microparticle.

In a thirtieth embodiment, alone or in combination with any other embodiment herein, the present disclosure relates to a hydrogel composition wherein the microparticle comprises a biodegradable polymer.

In a thirty-first embodiment, alone or in combination with any other embodiment herein, the present disclosure relates to a hydrogel composition wherein the biodegradable polymer is chosen from poly-glycolic acid (PGA), poly-lactic acid (PLA), poly-L-lactic acid (PLLA), polycaprolactone (PCL), poly-DL-lactic acid (PDLLA), poly(lactic-co-glycolic) acid (PLGA), poly(trimethylene carbonate) (PTMC), poly (ester amine)s (PEA), poly(para-dioxanone) (PPDO), poly-2-hydroxy butyrate (PHB), and co-polymers thereof.

In a thirty-second embodiment, alone or in combination with any other embodiment herein, the present disclosure relates to a hydrogel composition wherein the microparticle further comprises a therapeutic agent.

In a thirty-third embodiment, alone or in combination with any other embodiment herein, the present disclosure relates to a hydrogel composition wherein the microparticle further comprises a radioactive isotope or a compound including at least one radioactive element.

In a thirty-fourth embodiment, alone or in combination with any other embodiment herein, the present disclosure relates to a hydrogel composition wherein the radioactive isotope comprises yttrium-90.

In a thirty-fifth embodiment, alone or in combination with any other embodiment herein, the present disclosure relates to a hydrogel composition wherein the compound is chosen from yttrium phosphate (90YPO4), yttrium sulfate (90Y2 (SO4)3) or (89Y90Y(SO4)3), or yttrium carbonate (90Y2 (CO3)3) or (89Y90Y (CO3)3).

In a thirty-sixth embodiment, alone or in combination with any other embodiment herein, the present disclosure relates to a hydrogel composition wherein the microparticle further comprises a radiopaque marker.

In a thirty-seventh embodiment, alone or in combination with any other embodiment herein, the present disclosure relates to a hydrogel composition wherein the radiopaque marker comprises methylated TIBA (TIBA-Me).

In a thirty-eighth embodiment, alone or in combination with any other embodiment herein, the present disclosure relates to a hydrogel composition wherein the embedded component comprises a tissue marker.

In a thirty-ninth embodiment, alone or in combination with any other embodiment herein, the present disclosure relates to a hydrogel composition wherein the tissue marker is chosen from titanium particles, nitinol particles, PVA particles, or combinations thereof.

In a fortieth embodiment, alone or in combination with any other embodiment herein, the present disclosure relates to a hydrogel composition wherein the hydrogel is comprised of a first layer and a second layer.

In a forty-first embodiment, alone or in combination with any other embodiment herein, the present disclosure relates to a method to prepare a hydrogel comprising: preparing a first solution of a monomeric unit reconstituted in a first aqueous solution; preparing a second solution of a complexing molecule reconstituted in a second aqueous solution; and contacting the first solution with the second solution as both are poured into a cavity space in a subject to form a polymerized complex therein.

In a forty-second embodiment, alone or in combination with any other embodiment herein, the present disclosure relates to a method to prepare a hydrogel wherein the cavity space is between the subject's rectum and prostate.

In a forty-third embodiment, alone or in combination with any other embodiment herein, the present disclosure relates to a method to prepare a hydrogel wherein the cavity space is located in the subject's breast tissue.

In a forty-fourth embodiment, alone or in combination with any other embodiment herein, the present disclosure relates to a method to prepare a hydrogel wherein the complexing molecule is rHA or PEI.

In a forty-fifth embodiment, alone or in combination with any other embodiment herein, the present disclosure relates to a method to prepare a hydrogel wherein the monomeric unit comprises a terminal structure, a linker and a core and wherein the complexing molecule is chosen from recombinant albumin, polyethyleneimine (PEI), and poly-lysine.

In a forty-sixth embodiment, alone or in combination with any other embodiment herein, the present disclosure relates to a method to prepare a hydrogel wherein the linker comprises polyethylene glycol (PEG).

In a forty-seventh embodiment, alone or in combination with any other embodiment herein, the present disclosure relates to a method to prepare a hydrogel wherein the linker is of sufficient length to provide the hydrogel with a molecular weight (MW) of between 1 kDa and 100 kDa.

In a forty-eighth embodiment, alone or in combination with any other embodiment herein, the present disclosure relates to a method to prepare a hydrogel wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a forty-ninth embodiment, alone or in combination with any other embodiment herein, the present disclosure relates to a method to prepare a hydrogel wherein the core is chosen from pentaerythritol, hexaglycerol, tripentaerythritol, or glycerol.

In a fiftieth embodiment, alone or in combination with any other embodiment herein, the present disclosure relates to a method to prepare a hydrogel wherein the monomeric unit has 2, 4, or 8 linker arms extending from the core.

In a fifty-first embodiment, alone or in combination with any other embodiment herein, the present disclosure relates to a method to prepare a hydrogel wherein an ester of the monomeric unit complexes with an amine or imine of the complexing molecule.

In a fifty-second embodiment, alone or in combination with any other embodiment herein, the present disclosure relates to a method to prepare a hydrogel wherein a molar ratio of ester of the monomeric unit to amine or imine of the complexing molecule is of from 0.05 to 3.

In a fifty-third embodiment, alone or in combination with any other embodiment herein, the present disclosure relates to a method to prepare a hydrogel wherein the molar ratio is of 1.5 to 2.

In a fifty-fourth embodiment, alone or in combination with any other embodiment herein, the present disclosure relates to a method to prepare a hydrogel wherein the complexing molecule comprises from 2 to 60% weight/volume (w/v) of the hydrogel.

In a fifty-fifth embodiment, alone or in combination with any other embodiment herein, the present disclosure relates to a method to prepare a hydrogel wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a fifty-sixth embodiment, alone or in combination with any other embodiment herein, the present disclosure relates to a method to prepare a hydrogel wherein the monomeric unit is NHS-PEG-SG-8, NHS-PEG-SG-4, or NHS-PEG-SG-2.

In a fifty-seventh embodiment, alone or in combination with any other embodiment herein, the present disclosure relates to a method to prepare a hydrogel wherein the monomeric unit is NHS-PEG-SS-8, NHS-PEG-SS-4, or NHS-PEG-SS-2.

In a fifty-eighth embodiment, alone or in combination with any other embodiment herein, the present disclosure relates to a method to prepare a hydrogel wherein the monomeric unit is NHS-PEG-SCM-8, NHS-PEG-SCM-4, or NHS-PEG-SCM-2.

In a fifty-ninth embodiment, alone or in combination with any other embodiment herein, the present disclosure relates to a method to prepare a hydrogel wherein the complexing molecule is rHA.

In a sixtieth embodiment, alone or in combination with any other embodiment herein, the present disclosure relates to a method to prepare a hydrogel wherein the complexing molecule is PEI.

In a sixty-first embodiment, alone or in combination with any other embodiment herein, the present disclosure relates to a method to prepare a hydrogel further comprising a component to be embedded within the hydrogel within the first solution and/or the second solution.

In a sixty-second embodiment, alone or in combination with any other embodiment herein, the present disclosure relates to a method to prepare a hydrogel wherein the hydrogel has a pH of between 7.5 and 11.0.

In a sixty-third embodiment, alone or in combination with any other embodiment herein, the present disclosure relates to use of a hydrogel composition to separate rectal tissue from prostate tissue in a subject.

In a sixty-fourth embodiment, alone or in combination with any other embodiment herein, the present disclosure relates to use of a hydrogel composition wherein the cavity space is located between a targeted tissue and non-targeted tissue.

In a sixty-fifth embodiment, alone or in combination with any other embodiment herein, the present disclosure relates to use of a hydrogel composition wherein the cavity space is located between the rectum and the prostate.

In a sixty-sixth embodiment, alone or in combination with any other embodiment herein, the present disclosure relates to a kit comprising a first aqueous solution, a second aqueous solution, a dried monomeric unit, and a dried complexing molecule, wherein the first aqueous solution reconstitutes the monomeric unit and the second aqueous solution reconstitutes the complexing molecule.

In a sixty-seventh embodiment, alone or in combination with any other embodiment herein, the present disclosure relates to a kit further comprising a means to mix the first aqueous solution and the second aqueous solution as prior to application into a cavity space within a subject.

In a sixty-eighth embodiment, alone or in combination with any other embodiment herein, the present disclosure relates to a kit wherein the means comprises a first syringe barrel and a second syringe barrel both operably connected to a single needle point.

In a sixty-ninth embodiment, alone or in combination with any other embodiment herein, the present disclosure relates to a hydrogel composition for in situ formation within a cavity, comprising a polymerized complex of at least one monomeric unit, at least one complexing molecule, at least one osmotic component, and an aqueous solution, wherein the monomeric unit comprises a terminal structure, a linker and a core and wherein the complexing molecule is chosen from recombinant albumin, polyethyleneimine (PEI), and poly-lysine.

In a seventieth embodiment, alone or in combination with any other embodiment herein, the present disclosure relates to a hydrogel composition wherein the linker comprises polyethylene glycol (PEG).

In a seventy-first embodiment, alone or in combination with any other embodiment herein, the present disclosure relates to a hydrogel composition wherein the linker is of sufficient length to provide the hydrogel with a molecular weight (MW) of between 1 kDa and 100 kDa.

In a seventy-second embodiment, alone or in combination with any other embodiment herein, the present disclosure relates to a hydrogel composition wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a seventy-third embodiment, alone or in combination with any other embodiment herein, the present disclosure relates to a hydrogel composition wherein the core is chosen from pentaerythritol, hexaglycerol, tripentaerythritol, or glycerol.

In a seventy-fourth embodiment, alone or in combination with any other embodiment herein, the present disclosure relates to a hydrogel composition wherein the monomeric unit has 2, 4, or 8 linker arms extending from the core.

In a seventy-fifth embodiment, alone or in combination with any other embodiment herein, the present disclosure relates to a hydrogel composition wherein an ester of the monomeric unit complexes with an amine or imine of the complexing molecule.

In a seventy-sixth embodiment, alone or in combination with any other embodiment herein, the present disclosure relates to a hydrogel composition wherein a molar ratio of ester of the monomeric unit to amine or imine of the complexing molecule is of from 0.05 to 3.

In a seventy-seventh embodiment, alone or in combination with any other embodiment herein, the present disclosure relates to a hydrogel composition wherein the molar ratio is of 1.5 to 2.

In a seventy-eighth embodiment, alone or in combination with any other embodiment herein, the present disclosure relates to a hydrogel composition wherein the complexing molecule comprises from 2 to 60% weight/volume (w/v) of the hydrogel.

In a seventy-ninth embodiment, alone or in combination with any other embodiment herein, the present disclosure relates to a hydrogel composition wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In an eightieth embodiment, alone or in combination with any other embodiment herein, the present disclosure relates to a hydrogel composition wherein the hydrogel has a pH of between 7.5 and 11.0.

In an eighty-first embodiment, alone or in combination with any other embodiment herein, the present disclosure relates to a hydrogel composition wherein the monomeric unit is NHS-PEG-SG-8, NHS-PEG-SG-4, or NHS-PEG-SG-2.

In an eighty-second embodiment, alone or in combination with any other embodiment herein, the present disclosure relates to a hydrogel composition wherein the monomeric unit is NHS-PEG-SS-8, NHS-PEG-SS-4, or NHS-PEG-SS-2.

In an eighty-third embodiment, alone or in combination with any other embodiment herein, the present disclosure relates to a hydrogel composition wherein the monomeric unit is NHS-PEG-SCM-8, NHS-PEG-SCM-4, or NHS-PEG-SCM-2.

In an eighty-fourth embodiment, alone or in combination with any other embodiment herein, the present disclosure relates to a hydrogel composition wherein the complexing molecule is rHA.

In an eighty-fifth embodiment, alone or in combination with any other embodiment herein, the present disclosure relates to a hydrogel composition wherein the complexing molecule is PEI.

In an eighty-sixth embodiment, alone or in combination with any other embodiment herein, the present disclosure relates to a hydrogel composition wherein the osmotic component comprises one or more components chosen from: reverse osmosis water, salts, organic acids and/or acidic salts thereof, carbohydrates, monohydric and/or polyhydric alcohols, amino acids and/or peptides and/or proteins, and/or biodegradable polymers.

In an eighty-seventh embodiment, alone or in combination with any other embodiment herein, the present disclosure relates to a hydrogel composition wherein the salt is chosen from sodium chloride, calcium chloride, potassium chloride, magnesium chloride, sodium bicarbonate, sodium phosphate, sodium sulfate, potassium phosphate, potassium sulfate, calcium phosphate, ammonium sulfate, barium chloride, copper sulfate, ferric chloride, ferrous sulfate, lithium chloride, magnesium sulfate, manganese sulfate, nickel sulfate, potassium carbonate, potassium bromide, potassium chloride, potassium iodide, silver nitrate, sodium bromide, sodium carbonate, sodium chlorate, sodium nitrate, sodium pyrophosphate, zinc sulfate, and combinations thereof.

In an eighty-eighth embodiment, alone or in combination with any other embodiment herein, the present disclosure relates to a hydrogel composition wherein the carbohydrates are chosen dextrose, fructose, sucrose, lactose, maltose, trehalose, galactose, dextran, xylose, mannose, ribose, isomaltase, inulin, cyclodextrin, hydroxyethyl starch (HES), pullulan, pectin, xanthan gum, agarose, cellulose, hyaluronic acid, maltodextrin, methylcellulose, alginate, chitosan, heparin, heparan sulfate, and combinations thereof.

In an eighty-ninth embodiment, alone or in combination with any other embodiment herein, the present disclosure relates to a hydrogel composition wherein the organic acid and/or acidic salts thereof are chosen from fumaric acid, formic acid, gluconic acid, lactic acid, glycolic acid, pyruvic acid, oxalic acid, benzoic acid, cinnamic acid, ferulic acid, butyric acid, propionic acid, gallic acid, itaconic acid, maleic acid, mandelic acid, nicotinic acid, phthalic acid, salicylic acid, shikimic acid, uric acid, valeric acid, and combinations thereof.

In a ninetieth embodiment, alone or in combination with any other embodiment herein, the present disclosure relates to a hydrogel composition is a hypertonic hydrogel and has an osmotic pressure greater than 300 mOsm/L.

In a ninety-first embodiment, alone or in combination with any other embodiment herein, the present disclosure relates to a hydrogel composition wherein the hydrogel is a hypotonic hydrogel and has an osmotic pressure less than 280 mOsm/L.

In a ninety-second embodiment, alone or in combination with any other embodiment herein, the present disclosure relates to a hydrogel composition wherein the hydrogel is an isotonic hydrogel and has an osmotic pressure of between 280 mOsm/L and 300 mOsm/L.

In a ninety-third embodiment, alone or in combination with any other embodiment herein, the present disclosure relates to a hydrogel composition wherein the hydrogel further comprises a radiopaque marker.

In a ninety-fourth embodiment, alone or in combination with any other embodiment herein, the present disclosure relates to a hydrogel composition wherein the radiopaque marker comprises methylated TIBA (TIBA-Me).

In a ninety-fifth embodiment, alone or in combination with any other embodiment herein, the present disclosure relates to a hydrogel composition wherein the hydrogel is comprised of a first layer and a second layer.

In a ninety-sixth embodiment, alone or in combination with any other embodiment herein, the present disclosure relates to a hydrogel composition wherein the first layer has a first osmotic pressure and the second layer has a second osmotic pressure, different from the first osmotic pressure.

In a ninety-seventh embodiment, alone or in combination with any other embodiment herein, the present disclosure relates to a hydrogel composition wherein the first layer is hypertonic and the second layer is isotonic.

In a ninety-eighth embodiment, alone or in combination with any other embodiment herein, the present disclosure relates to a hydrogel composition wherein the first layer is first layer is hypotonic and the second layer is isotonic.

In a ninety-ninth embodiment, alone or in combination with any other embodiment herein, the present disclosure relates to a hydrogel composition wherein the first layer and/or the second layer comprises a therapeutic element.

In a one hundredth embodiment, alone or in combination with any other embodiment herein, the present disclosure relates to a method to prepare a hydrogel comprising: preparing a first solution of a monomeric unit reconstituted in a first aqueous solution; preparing a second solution of a complexing molecule reconstituted in a second aqueous solution; wherein an osmotic component is present in the first solution and/or the second solution; and contacting the first solution with the second solution as both are poured into a cavity space in a subject to form a polymerized complex therein.

In a one hundred and first embodiment, alone or in combination with any other embodiment herein, the present disclosure relates to a method to prepare a hydrogel wherein the cavity space is between the subject's rectum and prostate.

In a one hundred and second embodiment, alone or in combination with any other embodiment herein, the present disclosure relates to a method to prepare a hydrogel wherein the cavity space is located in the subject's breast tissue.

In a one hundred and third embodiment, alone or in combination with any other embodiment herein, the present disclosure relates to a method to prepare a hydrogel wherein the complexing molecule is rHA or PEI.

In a one hundred and fourth embodiment, alone or in combination with any other embodiment herein, the present disclosure relates to a method to prepare a hydrogel wherein the monomeric unit comprises a terminal structure, a linker and a core and wherein the complexing molecule is chosen from recombinant albumin, polyethyleneimine (PEI), and poly-lysine.

In a one hundred and fifth embodiment, alone or in combination with any other embodiment herein, the present disclosure relates to a method to prepare a hydrogel wherein the linker comprises polyethylene glycol (PEG).

In a one hundred and sixth embodiment, alone or in combination with any other embodiment herein, the present disclosure relates to a method to prepare a hydrogel wherein the linker is of sufficient length to provide the hydrogel with a molecular weight (MW) of between 1 kDa and 100 kDa.

In a one hundred and seventh embodiment, alone or in combination with any other embodiment herein, the present disclosure relates to a method to prepare a hydrogel wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a one hundred and eighth embodiment, alone or in combination with any other embodiment herein, the present disclosure relates to a method to prepare a hydrogel wherein the core is chosen from pentaerythritol, hexaglycerol, tripentaerythritol, or glycerol.

In a one hundred and ninth embodiment, alone or in combination with any other embodiment herein, the present disclosure relates to a method to prepare a hydrogel wherein the monomeric unit has 2, 4, or 8 linker arms extending from the core.

In a one hundred and tenth embodiment, alone or in combination with any other embodiment herein, the present disclosure relates to a method to prepare a hydrogel wherein an ester of the monomeric unit complexes with an amine or imine of the complexing molecule.

In a one hundred and eleventh embodiment, alone or in combination with any other embodiment herein, the present disclosure relates to a method to prepare a hydrogel wherein a molar ratio of ester of the monomeric unit to amine or imine of the complexing molecule is of from 0.05 to 3.

In a one hundred and twelfth embodiment, alone or in combination with any other embodiment herein, the present disclosure relates to a method to prepare a hydrogel wherein the molar ratio is of 1.5 to 2.

In a one hundred and thirteenth embodiment, alone or in combination with any other embodiment herein, the present disclosure relates to a method to prepare a hydrogel wherein the complexing molecule comprises from 2 to 60% weight/volume (w/v) of the hydrogel.

In a one hundred and fourteenth embodiment, alone or in combination with any other embodiment herein, the present disclosure relates to a method to prepare a hydrogel wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a one hundred and fifteenth embodiment, alone or in combination with any other embodiment herein, the present disclosure relates to a method to prepare a hydrogel wherein the hydrogel has a pH of between 7.5 and 11.0.

In a one hundred and sixteenth embodiment, alone or in combination with any other embodiment herein, the present disclosure relates to a method to prepare a hydrogel wherein the monomeric unit is NHS-PEG-SG-8, NHS-PEG-SG-4, or NHS-PEG-SG-2.

In a one hundred and seventeenth embodiment, alone or in combination with any other embodiment herein, the present disclosure relates to a method to prepare a hydrogel wherein the monomeric unit is NHS-PEG-SS-8, NHS-PEG-SS-4, or NHS-PEG-SS-2.

In a one hundred and eighteenth embodiment, alone or in combination with any other embodiment herein, the present disclosure relates to a method to prepare a hydrogel wherein the monomeric unit is NHS-PEG-SCM-8, NHS-PEG-SCM-4, or NHS-PEG-SCM-2.

In a one hundred and nineteenth embodiment, alone or in combination with any other embodiment herein, the present disclosure relates to a method to prepare a hydrogel wherein the complexing molecule is rHA.

In a one hundred and twentieth embodiment, alone or in combination with any other embodiment herein, the present disclosure relates to a method to prepare a hydrogel wherein the complexing molecule is PEI.

In a one hundred and twenty-first embodiment, alone or in combination with any other embodiment herein, the present disclosure relates to a method to prepare a hydrogel wherein the osmotic component comprises one or more components chosen from: reverse osmosis water, salts, organic acids and/or acidic salts thereof, carbohydrates, monohydric and/or polyhydric alcohols, amino acids and/or peptides and/or proteins, and/or biodegradable polymers.

In a one hundred and twenty-second embodiment, alone or in combination with any other embodiment herein, the present disclosure relates to a method to prepare a hydrogel wherein the hydrogel is comprised of a first layer and a second layer.

In a one hundred and twenty-third embodiment, alone or in combination with any other embodiment herein, the present disclosure relates to a method to prepare a hydrogel wherein the first layer has a first osmotic pressure and the second layer has a second osmotic pressure, different from the first osmotic pressure.

In a one hundred and twenty-fourth embodiment, alone or in combination with any other embodiment herein, the present disclosure relates to a method to prepare a hydrogel wherein the first layer is hypertonic and the second layer is isotonic.

In a one hundred and twenty-fifth embodiment, alone or in combination with any other embodiment herein, the present disclosure relates to a method to prepare a hydrogel wherein the first layer is first layer is hypotonic and the second layer is isotonic.

In a one hundred and twenty-sixth embodiment, alone or in combination with any other embodiment herein, the present disclosure relates to use of a hydrogel composition to separate a targeted tissue from a non-targeted tissue in a subject, wherein the osmotic component of the hydrogel generates an osmotic imbalance with the targeted tissue.

In a one hundred and twenty-seventh embodiment, alone or in combination with any other embodiment herein, the present disclosure relates to use of a hydrogel composition wherein the hypertonic hydrogel causes cytotoxic effects on the targeted tissue.

In a one hundred and twenty-eighth embodiment, alone or in combination with any other embodiment herein, the present disclosure relates to use of a hydrogel composition wherein the hypertonic first layer causes cytotoxic effects on the targeted tissue, but the isotonic second layer protects the non-targeted tissue.

In a one hundred and twenty-ninth embodiment, alone or in combination with any other embodiment herein, the present disclosure relates to use of a hydrogel composition wherein the hypotonic hydrogel causes lysis of the targeted tissue.

In a one hundred and thirtieth embodiment, alone or in combination with any other embodiment herein, the present disclosure relates to use of a hydrogel composition wherein the hypotonic first layer causes lysis of the targeted tissue, but the isotonic second layer protects the non-targeted tissue.

In a one hundred and thirty-first embodiment, alone or in combination with any other embodiment herein, the present disclosure relates to use of a hydrogel composition wherein the targeted tissue is prostate tissue.

In a one hundred and thirty-second embodiment, alone or in combination with any other embodiment herein, the present disclosure relates to use of a hydrogel composition wherein the non-targeted tissue is rectal tissue.

In a one hundred and thirty-third embodiment, alone or in combination with any other embodiment herein, the present disclosure relates to a kit comprising a first aqueous solution, a second aqueous solution, a dried monomeric unit, a dried complexing molecule, and one or more osmotic components wherein the first aqueous solution reconstitutes the monomeric unit and the second aqueous solution reconstitutes the complexing molecule.

In a one hundred and thirty-fourth embodiment, alone or in combination with any other embodiment herein, the present disclosure relates to a kit further comprising a means to mix the first aqueous solution and the second aqueous solution as prior to application into a cavity space within a subject.

In a one hundred and thirty-fifth embodiment, alone or in combination with any other embodiment herein, the present disclosure relates to a kit wherein the means comprises a first syringe barrel and a second syringe barrel both operably connected to a single needle point.

In a one hundred and thirty-sixth embodiment, alone or in combination with any other embodiment herein, the present disclosure relates to a hydrogel composition for in situ formation within a cavity, comprising a polymerized complex of at least one monomeric unit, at least one complexing molecule, a microparticle, and an aqueous solution, wherein the monomeric unit comprises a terminal structure, a linker and a core and wherein the complexing molecule is chosen from recombinant albumin, polyethyleneimine (PEI), and poly-lysine.

In a one hundred and thirty-seventh embodiment, alone or in combination with any other embodiment herein, the present disclosure relates to a hydrogel composition wherein the linker comprises polyethylene glycol (PEG).

In a one hundred and thirty-eighth embodiment, alone or in combination with any other embodiment herein, the present disclosure relates to a hydrogel composition wherein the linker is of sufficient length to provide the hydrogel with a molecular weight (MW) of between 1 kDa and 100 kDa.

In a one hundred and thirty-ninth embodiment, alone or in combination with any other embodiment herein, the present disclosure relates to a hydrogel composition wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a one hundred and fortieth embodiment, alone or in combination with any other embodiment herein, the present disclosure relates to a hydrogel composition wherein the core is chosen from pentaerythritol, hexaglycerol, tripentaerythritol, or glycerol.

In a one hundred and forty-first embodiment, alone or in combination with any other embodiment herein, the present disclosure relates to a hydrogel composition wherein the monomeric unit has 2, 4, or 8 linker arms extending from the core.

In a one hundred and forty-second embodiment, alone or in combination with any other embodiment herein, the present disclosure relates to a hydrogel composition wherein an ester of the monomeric unit complexes with an amine or imine of the complexing molecule.

In a one hundred and forty-third embodiment, alone or in combination with any other embodiment herein, the present disclosure relates to a hydrogel composition wherein a molar ratio of ester of the monomeric unit to amine or imine of the complexing molecule is of from 0.05 to 3.

In a one hundred and forty-fourth embodiment, alone or in combination with any other embodiment herein, the present disclosure relates to a hydrogel composition wherein the complexing molecule comprises from 2 to 60% weight/volume (w/v) of the hydrogel.

In a one hundred and forty-fifth embodiment, alone or in combination with any other embodiment herein, the present disclosure relates to a hydrogel composition wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a one hundred and forty-sixth embodiment, alone or in combination with any other embodiment herein, the present disclosure relates to a hydrogel composition wherein the hydrogel has a pH of between 7.5 and 11.0.

In a one hundred and forty-seventh embodiment, alone or in combination with any other embodiment herein, the present disclosure relates to a hydrogel composition wherein the monomeric unit is NHS-PEG-SG-8, NHS-PEG-SG-4, or NHS-PEG-SG-2.

In a one hundred and forty-eighth embodiment, alone or in combination with any other embodiment herein, the present disclosure relates to a hydrogel composition wherein the monomeric unit is NHS-PEG-SS-8, NHS-PEG-SS-4, or NHS-PEG-SS-2.

In a one hundred and forty-ninth embodiment, alone or in combination with any other embodiment herein, the present disclosure relates to a hydrogel composition wherein the monomeric unit is NHS-PEG-SCM-8, NHS-PEG-SCM-4, or NHS-PEG-SCM-2.

In a one hundred and fiftieth embodiment, alone or in combination with any other embodiment herein, the present disclosure relates to a hydrogel composition wherein the complexing molecule is rHA.

In a one hundred and fifty-first embodiment, alone or in combination with any other embodiment herein, the present disclosure relates to a hydrogel composition wherein the complexing molecule is PEI.

In a one hundred and fifty-second embodiment, alone or in combination with any other embodiment herein, the present disclosure relates to a hydrogel composition wherein the microparticle comprises a biodegradable polymer.

In a one hundred and fifty-third embodiment, alone or in combination with any other embodiment herein, the present disclosure relates to a hydrogel composition wherein the biodegradable polymer is chosen from poly-glycolic acid (PGA), poly-lactic acid (PLA), poly-L-lactic acid (PLLA), polycaprolactone (PCL), poly-DL-lactic acid (PDLLA), poly(lactic-co-glycolic) acid (PLGA), poly(trimethylene carbonate) (PTMC), poly (ester amine)s (PEA), poly(para-dioxanone) (PPDO), poly-2-hydroxy butyrate (PHB), and co-polymers thereof.

In a one hundred and fifty-fourth embodiment, alone or in combination with any other embodiment herein, the present disclosure relates to a hydrogel composition wherein the microparticle further comprises a therapeutic agent.

In a one hundred and fifty-fifth embodiment, alone or in combination with any other embodiment herein, the present disclosure relates to a hydrogel composition wherein the therapeutic agent is a chemotherapeutic agent chosen from docetaxel, degarelix, abiraterone, apalutamide, bicalutamide, cabazitaxel, darolutamide, leuprolide, enzalutamide, flutamide, goserelin, lutetium Lu177 vipivotide, tetraxetan, olaparib, mitoxantrone, nilutamide, relugolix, sipuleucel-T, radium 223, rucaparib camsylate, or combinations thereof.

In a one hundred and fifty-sixth embodiment, alone or in combination with any other embodiment herein, the present disclosure relates to a hydrogel composition wherein the therapeutic agent is a chemotherapeutic agent chosen from raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

In a one hundred and fifty-seventh embodiment, alone or in combination with any other embodiment herein, the present disclosure relates to a hydrogel composition wherein the therapeutic agent comprises a radiosensitizing agent chosen from gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, xinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

In a one hundred and fifty-eighth embodiment, alone or in combination with any other embodiment herein, the present disclosure relates to a hydrogel composition wherein the therapeutic agent comprises a radioprotective agent chosen from amifostine, palifermin, super oxide dismutase, tetracycline, genistein, captopril, Lisinopril, 3,3′-diindolylmethane, rapamycin, CBLB502, ON01210, γ-tocotrienol, δ-tocotrienol, R-spondin 1, transforming growth factor β3, mesenchymal stem cells, bone marrow stromal cells, myeloid progenitor cells, antioxidants, or combinations thereof.

In a one hundred and fifty-ninth embodiment, alone or in combination with any other embodiment herein, the present disclosure relates to a hydrogel composition wherein the microparticle further comprises a radioactive isotope or a compound including at least one radioactive element.

In a one hundred and sixtieth embodiment, alone or in combination with any other embodiment herein, the present disclosure relates to a hydrogel composition wherein the radioactive isotope comprises yttrium-90.

In a one hundred and sixty-first embodiment, alone or in combination with any other embodiment herein, the present disclosure relates to a hydrogel composition wherein the compound is chosen from yttrium phosphate (90YPO4), yttrium sulfate (90Y2 (SO4)3) or (89Y90Y(SO4)3), or yttrium carbonate (90Y2 (CO3)3) or (89Y90Y(CO3)3).

In a one hundred and sixty-second embodiment, alone or in combination with any other embodiment herein, the present disclosure relates to a hydrogel composition wherein the microparticle further comprises a radiopaque marker.

In a one hundred and sixty-third embodiment, alone or in combination with any other embodiment herein, the present disclosure relates to a hydrogel composition wherein the radiopaque marker comprises methylated TIBA (TIBA-Me).

In a one hundred and sixty-fourth embodiment, alone or in combination with any other embodiment herein, the present disclosure relates to a hydrogel composition wherein the hydrogel is comprised of a first layer and a second layer.

In a one hundred and sixty-fifth embodiment, alone or in combination with any other embodiment herein, the present disclosure relates to a hydrogel composition wherein the microparticle is absent from the second layer.

In a one hundred and sixty-sixth embodiment, alone or in combination with any other embodiment herein, the present disclosure relates to a hydrogel composition wherein the second layer comprises a second microparticle and further wherein the second microparticle differs in composition from the microparticle.

In a one hundred and sixty-seventh embodiment, alone or in combination with any other embodiment herein, the present disclosure relates to a method to prepare a hydrogel comprising: preparing a first solution of a monomeric unit reconstituted in a first aqueous solution; preparing a second solution of a complexing molecule reconstituted in a second aqueous solution; suspending a microparticle in the first solution and/or second solution; and contacting the first solution with the second solution as both are poured into a cavity space in a subject to form a polymerized complex therein.

In a one hundred and sixty-eighth embodiment, alone or in combination with any other embodiment herein, the present disclosure relates to a method to prepare a hydrogel wherein the cavity space is between the subject's rectum and prostate.

In a one hundred and sixty-ninth embodiment, alone or in combination with any other embodiment herein, the present disclosure relates to a method to prepare a hydrogel wherein the cavity space is located in the subject's breast tissue.

In a one hundred and seventieth embodiment, alone or in combination with any other embodiment herein, the present disclosure relates to a method to prepare a hydrogel wherein the complexing molecule is rHA or PEI.

In a one hundred and seventy-first embodiment, alone or in combination with any other embodiment herein, the present disclosure relates to a method to prepare a hydrogel wherein the monomeric unit comprises a terminal structure, a linker and a core and wherein the complexing molecule is chosen from recombinant albumin, polyethyleneimine (PEI), and poly-lysine.

In a one hundred and seventy-second embodiment, alone or in combination with any other embodiment herein, the present disclosure relates to a method to prepare a hydrogel wherein the linker comprises polyethylene glycol (PEG).

In a one hundred and seventy-third embodiment, alone or in combination with any other embodiment herein, the present disclosure relates to a method to prepare a hydrogel wherein the linker is of sufficient length to provide the hydrogel with a molecular weight (MW) of between 1 kDa and 100 kDa.

In a one hundred and seventy-fourth embodiment, alone or in combination with any other embodiment herein, the present disclosure relates to a method to prepare a hydrogel wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a one hundred and seventy-fifth embodiment, alone or in combination with any other embodiment herein, the present disclosure relates to a method to prepare a hydrogel wherein the core is chosen from pentaerythritol, hexaglycerol, tripentaerythritol, or glycerol.

In a one hundred and seventy-sixth embodiment, alone or in combination with any other embodiment herein, the present disclosure relates to a method to prepare a hydrogel wherein the monomeric unit has 2, 4, or 8 linker arms extending from the core.

In a one hundred and seventy-seventh embodiment, alone or in combination with any other embodiment herein, the present disclosure relates to a method to prepare a hydrogel wherein an ester of the monomeric unit complexes with an amine or imine of the complexing molecule.

In a one hundred and seventy-eighth embodiment, alone or in combination with any other embodiment herein, the present disclosure relates to a method to prepare a hydrogel wherein a molar ratio of ester of the monomeric unit to amine or imine of the complexing molecule is of from 0.05 to 3.

In a one hundred and seventy-ninth embodiment, alone or in combination with any other embodiment herein, the present disclosure relates to a method to prepare a hydrogel wherein the molar ratio is of 1.5 to 2.

In a one hundred and eightieth embodiment, alone or in combination with any other embodiment herein, the present disclosure relates to a method to prepare a hydrogel wherein the complexing molecule comprises from 2 to 60% weight/volume (w/v) of the hydrogel.

In a one hundred and eighty-first embodiment, alone or in combination with any other embodiment herein, the present disclosure relates to a method to prepare a hydrogel wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a one hundred and eighty-second embodiment, alone or in combination with any other embodiment herein, the present disclosure relates to a method to prepare a hydrogel wherein the hydrogel has a pH of between 7.5 and 11.0.

In a one hundred and eighty-third embodiment, alone or in combination with any other embodiment herein, the present disclosure relates to a method to prepare a hydrogel wherein the monomeric unit is NHS-PEG-SG-8, NHS-PEG-SG-4, or NHS-PEG-SG-2.

In a one hundred and eighty-fourth embodiment, alone or in combination with any other embodiment herein, the present disclosure relates to a method to prepare a hydrogel wherein the monomeric unit is NHS-PEG-SS-8, NHS-PEG-SS-4, or NHS-PEG-SS-2.

In a one hundred and eighty-fifth embodiment, alone or in combination with any other embodiment herein, the present disclosure relates to a method to prepare a hydrogel wherein the monomeric unit is NHS-PEG-SCM-8, NHS-PEG-SCM-4, or NHS-PEG-SCM-2.

In a one hundred and eighty-sixth embodiment, alone or in combination with any other embodiment herein, the present disclosure relates to a method to prepare a hydrogel wherein the complexing molecule is rHA.

In a one hundred and eighty-seventh embodiment, alone or in combination with any other embodiment herein, the present disclosure relates to a method to prepare a hydrogel wherein the complexing molecule is PEI.

In a one hundred and eighty-eighth embodiment, alone or in combination with any other embodiment herein, the present disclosure relates to a method to prepare a hydrogel wherein the microparticle comprises a biodegradable polymer.

In a one hundred and eighty-ninth embodiment, alone or in combination with any other embodiment herein, the present disclosure relates to a method to prepare a hydrogel wherein the biodegradable polymer is chosen from poly-glycolic acid (PGA), poly-lactic acid (PLA), poly-L-lactic acid (PLLA), polycaprolactone (PCL), poly-DL-lactic acid (PDLLA), poly(lactic-co-glycolic) acid (PLGA), poly(trimethylene carbonate) (PTMC), poly (ester amine)s (PEA), poly(para-dioxanone) (PPDO), poly-2-hydroxy butyrate (PHB), and co-polymers thereof.

In a one hundred and ninetieth embodiment, alone or in combination with any other embodiment herein, the present disclosure relates to a method to prepare a hydrogel wherein the microparticle further comprises a therapeutic agent.

In a one hundred and ninety-first embodiment, alone or in combination with any other embodiment herein, the present disclosure relates to a method to prepare a hydrogel wherein the therapeutic agent is a chemotherapeutic agent chosen from docetaxel, degarelix, abiraterone, apalutamide, bicalutamide, cabazitaxel, darolutamide, leuprolide, enzalutamide, flutamide, goserelin, lutetium Lu177 vipivotide, tetraxetan, olaparib, mitoxantrone, nilutamide, relugolix, sipuleucel-T, radium 223, rucaparib camsylate, or combinations thereof.

In a one hundred and ninety-second embodiment, alone or in combination with any other embodiment herein, the present disclosure relates to a method to prepare a hydrogel wherein the therapeutic agent is a chemotherapeutic agent chosen from raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

In a one hundred and ninety-third embodiment, alone or in combination with any other embodiment herein, the present disclosure relates to a method to prepare a hydrogel wherein the therapeutic agent comprises a radiosensitizing agent chosen from gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, xinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

In a one hundred and ninety-fourth embodiment, alone or in combination with any other embodiment herein, the present disclosure relates to a method to prepare a hydrogel wherein the therapeutic agent comprises a radioprotective agent chosen from amifostine, palifermin, super oxide dismutase, tetracycline, genistein, captopril, Lisinopril, 3,3′-diindolylmethane, rapamycin, CBLB502, ON01210, γ-tocotrienol, 8-tocotrienol, R-spondin 1, transforming growth factor β3, mesenchymal stem cells, bone marrow stromal cells, myeloid progenitor cells, antioxidants, or combinations thereof.

In a one hundred and ninety-fifth embodiment, alone or in combination with any other embodiment herein, the present disclosure relates to a method to prepare a hydrogel wherein the microparticle further comprises a radioactive isotope or a compound including at least one radioactive element.

In a one hundred and ninety-sixth embodiment, alone or in combination with any other embodiment herein, the present disclosure relates to a method to prepare a hydrogel wherein the radioactive isotope comprises yttrium-90.

In a one hundred and ninety-seventh embodiment, alone or in combination with any other embodiment herein, the present disclosure relates to a method to prepare a hydrogel wherein the compound is chosen from yttrium phosphate (90YPO4), yttrium sulfate (90Y2 (SO4)3) or (89Y90Y(SO4)3), or yttrium carbonate (90Y2 (CO3)3) or (89Y90Y(CO3)3).

In a one hundred and ninety-eighth embodiment, alone or in combination with any other embodiment herein, the present disclosure relates to a method to prepare a hydrogel wherein the microparticle further comprises a radiopaque marker.

In a one hundred and ninety-ninth embodiment, alone or in combination with any other embodiment herein, the present disclosure relates to a method to prepare a hydrogel wherein the radiopaque marker comprises methylated TIBA (TIBA-Me).

In a two hundredth embodiment, alone or in combination with any other embodiment herein, the present disclosure relates to a method to prepare a hydrogel wherein the hydrogel is comprised of a first layer and a second layer.

In a two hundred and first embodiment, alone or in combination with any other embodiment herein, the present disclosure relates to use of a hydrogel composition to separate a targeted tissue from a non-targeted tissue in a subject, wherein the embedded component exerts a therapeutic effect on and/or enhances a treatment of the targeted tissue.

In a two hundred and second embodiment, alone or in combination with any other embodiment herein, the present disclosure relates to use of a hydrogel composition wherein the treatment of the targeted issue is radiation therapy.

In a two hundred and third embodiment, alone or in combination with any other embodiment herein, the present disclosure relates to use of a hydrogel composition wherein the targeted tissue is prostate tissue.

In a two hundred and fourth embodiment, alone or in combination with any other embodiment herein, the present disclosure relates to use of a hydrogel composition wherein the non-targeted tissue is rectal tissue.

In a two hundred and fifth embodiment, alone or in combination with any other embodiment herein, the present disclosure relates to a kit comprising a first aqueous solution, a second aqueous solution, a dried monomeric unit, and a dried complexing molecule, wherein the first aqueous solution reconstitutes the monomeric unit and the second aqueous solution reconstitutes the complexing molecule and one or more microparticles.

In a two hundred and sixth embodiment, alone or in combination with any other embodiment herein, the present disclosure relates to a kit comprising a means to mix the first aqueous solution and the second aqueous solution as prior to application into a cavity space within a subject.

In a two hundred and seventh embodiment, alone or in combination with any other embodiment herein, the present disclosure relates to a kit wherein the means comprises a first syringe barrel and a second syringe barrel both operably connected to a single needle point.

In a two hundred and eighth embodiment, alone or in combination with any other embodiment herein, the present disclosure relates to a hydrogel composition for in situ formation within a cavity, comprising a polymerized complex of at least one monomeric unit, at least one complexing molecule, an embedded component, and an aqueous solution, wherein the monomeric unit comprises a terminal structure, a linker and a core and wherein the complexing molecule is chosen from recombinant albumin, polyethyleneimine (PEI), and poly-lysine.

In a two hundred and ninth embodiment, alone or in combination with any other embodiment herein, the present disclosure relates to a hydrogel composition wherein the linker comprises polyethylene glycol (PEG).

In a two hundred and tenth embodiment, alone or in combination with any other embodiment herein, the present disclosure relates to a hydrogel composition wherein the linker is of sufficient length to provide the hydrogel with a molecular weight (MW) of between 1 kDa and 100 kDa.

In a two hundred and eleventh embodiment, alone or in combination with any other embodiment herein, the present disclosure relates to a hydrogel composition wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a two hundred and twelfth embodiment, alone or in combination with any other embodiment herein, the present disclosure relates to a hydrogel composition wherein the core is chosen from pentaerythritol, hexaglycerol, tripentaerythritol, or glycerol.

In a two hundred and thirteenth embodiment, alone or in combination with any other embodiment herein, the present disclosure relates to a hydrogel composition wherein the monomeric unit has 2, 4, or 8 linker arms extending from the core.

In a two hundred and fourteenth embodiment, alone or in combination with any other embodiment herein, the present disclosure relates to a hydrogel composition wherein an ester of the monomeric unit complexes with an amine or imine of the complexing molecule.

In a two hundred and fifteenth embodiment, alone or in combination with any other embodiment herein, the present disclosure relates to a hydrogel composition wherein a molar ratio of ester of the monomeric unit to amine or imine of the complexing molecule is of from 0.05 to 3.

In a two hundred and sixteenth embodiment, alone or in combination with any other embodiment herein, the present disclosure relates to a hydrogel composition wherein the molar ratio is of 1.5 to 2.

In a two hundred and seventeenth embodiment, alone or in combination with any other embodiment herein, the present disclosure relates to a hydrogel composition wherein the complexing molecule comprises from 2 to 60% weight/volume (w/v) of the hydrogel.

In a two hundred and eighteenth embodiment, alone or in combination with any other embodiment herein, the present disclosure relates to a hydrogel composition wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a two hundred and nineteenth embodiment, alone or in combination with any other embodiment herein, the present disclosure relates to a hydrogel composition wherein the hydrogel has a pH of between 7.5 and 11.0.

In a two hundred and twentieth embodiment, alone or in combination with any other embodiment herein, the present disclosure relates to a hydrogel composition wherein the monomeric unit is NHS-PEG-SG-8, NHS-PEG-SG-4, or NHS-PEG-SG-2.

In a two hundred and twenty-first embodiment, alone or in combination with any other embodiment herein, the present disclosure relates to a hydrogel composition wherein the monomeric unit is NHS-PEG-SS-8, NHS-PEG-SS-4, or NHS-PEG-SS-2.

In a two hundred and twenty-second embodiment, alone or in combination with any other embodiment herein, the present disclosure relates to a hydrogel composition wherein the monomeric unit is NHS-PEG-SCM-8, NHS-PEG-SCM-4, or NHS-PEG-SCM-2.

In a two hundred and twenty-third embodiment, alone or in combination with any other embodiment herein, the present disclosure relates to a hydrogel composition wherein the complexing molecule is rHA.

In a two hundred and twenty-fourth embodiment, alone or in combination with any other embodiment herein, the present disclosure relates to a hydrogel composition wherein the complexing molecule is PEI.

In a two hundred and twenty-fifth embodiment, alone or in combination with any other embodiment herein, the present disclosure relates to a hydrogel composition wherein the embedded component is dissolved within the hydrogel.

In a two hundred and twenty-sixth embodiment, alone or in combination with any other embodiment herein, the present disclosure relates to a hydrogel composition wherein the embedded component agent is a therapeutic agent.

In a two hundred and twenty-seventh embodiment, alone or in combination with any other embodiment herein, the present disclosure relates to a hydrogel composition wherein the therapeutic agent is a chemotherapeutic agent chosen from docetaxel, degarelix, abiraterone, apalutamide, bicalutamide, cabazitaxel, darolutamide, leuprolide, enzalutamide, flutamide, goserelin, lutetium Lu177 vipivotide, tetraxetan, olaparib, mitoxantrone, nilutamide, relugolix, sipuleucel-T, radium 223, rucaparib camsylate, or combinations thereof.

In a two hundred and twenty-eighth embodiment, alone or in combination with any other embodiment herein, the present disclosure relates to a hydrogel composition wherein the therapeutic agent is a chemotherapeutic agent chosen from raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

In a two hundred and twenty-ninth embodiment, alone or in combination with any other embodiment herein, the present disclosure relates to a hydrogel composition wherein the embedded component is a radiosensitizing agent chosen from gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, xinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

In a two hundred and thirtieth embodiment, alone or in combination with any other embodiment herein, the present disclosure relates to a hydrogel composition wherein the embedded component is a radioprotective agent chosen from amifostine, palifermin, super oxide dismutase, tetracycline, genistein, captopril, Lisinopril, 3,3′-diindolylmethane, rapamycin, CBLB502, ON01210, γ-tocotrienol, 8-tocotrienol, R-spondin 1, transforming growth factor β3, mesenchymal stem cells, bone marrow stromal cells, myeloid progenitor cells, antioxidants, or combinations thereof.

In a two hundred and thirty-first embodiment, alone or in combination with any other embodiment herein, the present disclosure relates to a hydrogel composition wherein the embedded component comprises a compound that allows for the production of reactive oxygen species from radiotherapy.

In a two hundred and thirty-second embodiment, alone or in combination with any other embodiment herein, the present disclosure relates to a hydrogel composition wherein the embedded component comprises a peroxide.

In a two hundred and thirty-third embodiment, alone or in combination with any other embodiment herein, the present disclosure relates to a hydrogel composition wherein the peroxide comprises formamide peroxide.

In a two hundred and thirty-fourth embodiment, alone or in combination with any other embodiment herein, the present disclosure relates to a hydrogel composition wherein the embedded component comprises a radiopaque compound.

In a two hundred and thirty-fifth embodiment, alone or in combination with any other embodiment herein, the present disclosure relates to a hydrogel composition wherein the radiopaque compound comprises methylated TIBA (TIBA-Me).

In a two hundred and thirty-sixth embodiment, alone or in combination with any other embodiment herein, the present disclosure relates to a hydrogel composition wherein the embedded component comprises a radioactive isotope or a compound including at least one radioactive element.

In a two hundred and thirty-seventh embodiment, alone or in combination with any other embodiment herein, the present disclosure relates to a hydrogel composition wherein the radioactive isotope comprises yttrium-90.

In a two hundred and thirty-eighth embodiment, alone or in combination with any other embodiment herein, the present disclosure relates to a hydrogel composition wherein the compound is chosen from yttrium phosphate (90YPO4), yttrium sulfate (90Y2 (SO4)3) or (89Y90Y(SO4)3), or yttrium carbonate (90Y2 (CO3)3) or (89Y90Y(CO3)3).

In a two hundred and thirty-ninth embodiment, alone or in combination with any other embodiment herein, the present disclosure relates to a hydrogel composition wherein the hydrogel is comprised of a first layer and a second layer.

In a two hundred and fortieth embodiment, alone or in combination with any other embodiment herein, the present disclosure relates to a hydrogel composition wherein the embedded component is absent from the second layer.

In a two hundred and forty-first embodiment, alone or in combination with any other embodiment herein, the present disclosure relates to a hydrogel composition wherein the first layer comprises a first embedded component and the second layer comprises a second embedded component.

In a two hundred and forty-second embodiment, alone or in combination with any other embodiment herein, the present disclosure relates to a hydrogel composition wherein the first layer comprises a first polymerized complex and the second layer comprises a second polymerized complex differing from the first polymerized complex by one or more of: the monomeric unit, the complexing molecule, the embedded component or the aqueous solution.

In a two hundred and forty-third embodiment, alone or in combination with any other embodiment herein, the present disclosure relates to a method to prepare a hydrogel comprising: preparing a first solution of a monomeric unit reconstituted in a first aqueous solution; preparing a second solution of a complexing molecule reconstituted in a second aqueous solution; wherein an embedded component is present in the first solution and/or the second solution; and contacting the first solution with the second solution as both are poured into a cavity space in a subject to form a polymerized complex therein.

In a two hundred and forty-fourth embodiment, alone or in combination with any other embodiment herein, the present disclosure relates to a method to prepare a hydrogel wherein the cavity space is between the subject's rectum and prostate.

In a two hundred and forty-fifth embodiment, alone or in combination with any other embodiment herein, the present disclosure relates to a method to prepare a hydrogel wherein the cavity space is located in the subject's breast tissue.

In a two hundred and forty-sixth embodiment, alone or in combination with any other embodiment herein, the present disclosure relates to a method to prepare a hydrogel wherein the complexing molecule is rHA or PEI.

In a two hundred and forty-seventh embodiment, alone or in combination with any other embodiment herein, the present disclosure relates to a method to prepare a hydrogel wherein the monomeric unit comprises a terminal structure, a linker and a core and wherein the complexing molecule is chosen from recombinant albumin, polyethyleneimine (PEI), and poly-lysine.

In a two hundred and forty-eighth embodiment, alone or in combination with any other embodiment herein, the present disclosure relates to a method to prepare a hydrogel wherein the linker comprises polyethylene glycol (PEG).

In a two hundred and forty-ninth embodiment, alone or in combination with any other embodiment herein, the present disclosure relates to a method to prepare a hydrogel wherein the linker is of sufficient length to provide the hydrogel with a molecular weight (MW) of between 1 kDa and 100 kDa.

In a two hundred and fiftieth embodiment, alone or in combination with any other embodiment herein, the present disclosure relates to a method to prepare a hydrogel wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a two hundred and fifty-first embodiment, alone or in combination with any other embodiment herein, the present disclosure relates to a method to prepare a hydrogel wherein the core is chosen from pentaerythritol, hexaglycerol, tripentaerythritol, or glycerol.

In a two hundred and fifty-second embodiment, alone or in combination with any other embodiment herein, the present disclosure relates to a method to prepare a hydrogel wherein the monomeric unit has 2, 4, or 8 linker arms extending from the core.

In a two hundred and fifty-third embodiment, alone or in combination with any other embodiment herein, the present disclosure relates to a method to prepare a hydrogel wherein an ester of the monomeric unit complexes with an amine or imine of the complexing molecule.

In a two hundred and fifty-fourth embodiment, alone or in combination with any other embodiment herein, the present disclosure relates to a method to prepare a hydrogel wherein a molar ratio of ester of the monomeric unit to amine or imine of the complexing molecule is of from 0.05 to 3.

In a two hundred and fifty-fifth embodiment, alone or in combination with any other embodiment herein, the present disclosure relates to a method to prepare a hydrogel wherein the molar ratio is of 1.5 to 2.

In a two hundred and fifty-sixth embodiment, alone or in combination with any other embodiment herein, the present disclosure relates to a method to prepare a hydrogel wherein the complexing molecule comprises from 2 to 60% weight/volume (w/v) of the hydrogel.

In a two hundred and fifty-seventh embodiment, alone or in combination with any other embodiment herein, the present disclosure relates to a method to prepare a hydrogel wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a two hundred and fifty-eighth embodiment, alone or in combination with any other embodiment herein, the present disclosure relates to a method to prepare a hydrogel wherein the hydrogel has a pH of between 7.5 and 11.0.

In a two hundred and fifty-ninth embodiment, alone or in combination with any other embodiment herein, the present disclosure relates to a method to prepare a hydrogel

In a two hundred and sixtieth embodiment, alone or in combination with any other embodiment herein, the present disclosure relates to a method to prepare a hydrogel wherein the monomeric unit is NHS-PEG-SG-8, NHS-PEG-SG-4, or NHS-PEG-SG-2.

In a two hundred and sixty-first embodiment, alone or in combination with any other embodiment herein, the present disclosure relates to a method to prepare a hydrogel wherein the monomeric unit is NHS-PEG-SS-8, NHS-PEG-SS-4, or NHS-PEG-SS-2.

In a two hundred and sixty-second embodiment, alone or in combination with any other embodiment herein, the present disclosure relates to a method to prepare a hydrogel wherein the monomeric unit is NHS-PEG-SCM-8, NHS-PEG-SCM-4, or NHS-PEG-SCM-2.

In a two hundred and sixty-third embodiment, alone or in combination with any other embodiment herein, the present disclosure relates to a method to prepare a hydrogel wherein the complexing molecule is rHA.

In a two hundred and sixty-fourth embodiment, alone or in combination with any other embodiment herein, the present disclosure relates to use of a hydrogel composition to separate a targeted tissue from a non-targeted tissue in a subject, wherein the embedded component exerts a therapeutic effect on and/or enhances a treatment of the targeted tissue.

In a two hundred and sixty-fifth embodiment, alone or in combination with any other embodiment herein, the present disclosure relates to use of a hydrogel composition wherein the treatment of the targeted tissue is radiation therapy.

In a two hundred and sixty-sixth embodiment, alone or in combination with any other embodiment herein, the present disclosure relates to use of a hydrogel composition wherein the targeted tissue is prostate tissue.

In a two hundred and sixty-seventh embodiment, alone or in combination with any other embodiment herein, the present disclosure relates to relates to use of a hydrogel composition wherein the non-targeted tissue is rectal tissue.

In a two hundred and sixty-eighth embodiment, alone or in combination with any other embodiment herein, the present disclosure relates to a kit comprising a first aqueous solution, a second aqueous solution, a dried monomeric unit, a dried complexing molecule, and one or more embedded components, wherein the first aqueous solution reconstitutes the monomeric unit and the second aqueous solution reconstitutes the complexing molecule.

In a two hundred and sixty-ninth embodiment, alone or in combination with any other embodiment herein, the present disclosure relates to a kit comprising a means to mix the first aqueous solution and the second aqueous solution as prior to application into a cavity space within a subject.

In a two hundred and seventieth embodiment, alone or in combination with any other embodiment herein, the present disclosure relates to a kit wherein the means comprises a first syringe barrel and a second syringe barrel both operably connected to a single needle point.

In a two hundred and seventy-first embodiment, alone or in combination with any other embodiment herein, the present disclosure relates to a hydrogel composition for in situ formation within a cavity, comprising a polymerized complex of at least one monomeric unit chosen from NHS-PEG-SCM-8, NHS-PEG-SCM-4, or NHS-PEG-SCM-2, at least one complexing molecule and an aqueous solution, wherein the complexing molecule is polyethyleneimine (PEI).

In a two hundred and seventy-second embodiment, alone or in combination with any other embodiment herein, the present disclosure relates to a hydrogel composition wherein an ester of the monomeric unit complexes with an amine or imine of the complexing molecule.

In a two hundred and seventy-third embodiment, alone or in combination with any other embodiment herein, the present disclosure relates to a hydrogel composition wherein a molar ratio of ester of the monomeric unit to amine or imine of the complexing molecule is of from 0.05 to 3.

In a two hundred and seventy-fourth embodiment, alone or in combination with any other embodiment herein, the present disclosure relates to a hydrogel composition wherein the molar ratio is of 1.5 to 2.

In a two hundred and seventy-fifth embodiment, alone or in combination with any other embodiment herein, the present disclosure relates to a hydrogel composition wherein the molar ratio is of from 1.75-2.

In a two hundred and seventy-sixth embodiment, alone or in combination with any other embodiment herein, the present disclosure relates to a hydrogel composition wherein the complexing molecule comprises from 2 to 60% weight/volume (w/v) of the hydrogel.

In a two hundred and seventy-seventh embodiment, alone or in combination with any other embodiment herein, the present disclosure relates to a hydrogel composition wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a two hundred and seventy-eighth embodiment, alone or in combination with any other embodiment herein, the present disclosure relates to a hydrogel composition wherein the hydrogel has a pH of between 7.5 and 11.0.

In a two hundred and seventy-ninth embodiment, alone or in combination with any other embodiment herein, the present disclosure relates to a hydrogel composition further comprising an embedded component within the hydrogel.

In a two hundred and eightieth embodiment, alone or in combination with any other embodiment herein, the present disclosure relates to a hydrogel composition wherein the embedded component is dissolved within the hydrogel.

In a two hundred and eighty-first embodiment, alone or in combination with any other embodiment herein, the present disclosure relates to a hydrogel composition wherein the embedded component is in a crystalline or amorphous solid form suspended throughout the hydrogel.

In a two hundred and eighty-second embodiment, alone or in combination with any other embodiment herein, the present disclosure relates to a hydrogel composition wherein the embedded component comprises a therapeutic agent.

In a two hundred and eighty-third embodiment, alone or in combination with any other embodiment herein, the present disclosure relates to a hydrogel composition wherein the therapeutic agent is a chemotherapeutic agent chosen from docetaxel, degarelix, abiraterone, apalutamide, bicalutamide, cabazitaxel, darolutamide, leuprolide, enzalutamide, flutamide, goserelin, lutetium Lu177 vipivotide, tetraxetan, olaparib, mitoxantrone, nilutamide, relugolix, sipuleucel-T, radium 223, rucaparib camsylate, or combinations thereof.

In a two hundred and eighty-fourth embodiment, alone or in combination with any other embodiment herein, the present disclosure relates to a hydrogel composition, wherein the therapeutic agent is a chemotherapeutic agent chosen from raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

In a two hundred and eighty-fifth embodiment, alone or in combination with any other embodiment herein, the present disclosure relates to a hydrogel composition wherein the embedded component is a radiosensitizing agent chosen from gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, xinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

In a two hundred and eighty-sixth embodiment, alone or in combination with any other embodiment herein, the present disclosure relates to a hydrogel composition wherein the embedded component is a radioprotective agent chosen from amifostine, palifermin, super oxide dismutase, tetracycline, genistein, captopril, Lisinopril, 3,3′-diindolylmethane, rapamycin, CBLB502, ON01210, γ-tocotrienol, δ-tocotrienol, R-spondin 1, transforming growth factor β3, mesenchymal stem cells, bone marrow stromal cells, myeloid progenitor cells, antioxidants, or combinations thereof.

In a two hundred and eighty-seventh embodiment, alone or in combination with any other embodiment herein, the present disclosure relates to a hydrogel composition wherein the embedded component comprises gas nanobubbles.

In a two hundred and eighty-eighth embodiment, alone or in combination with any other embodiment herein, the present disclosure relates to a hydrogel composition wherein the embedded component comprises a compound that allows for the production of reactive oxygen species from radiotherapy.

In a two hundred and eighty-ninth embodiment, alone or in combination with any other embodiment herein, the present disclosure relates to a hydrogel composition wherein the embedded component comprises a peroxide.

In a two hundred and ninetieth embodiment, alone or in combination with any other embodiment herein, the present disclosure relates to a hydrogel composition wherein the peroxide comprises formamide peroxide.

In a two hundred and ninety-first embodiment, alone or in combination with any other embodiment herein, the present disclosure relates to a hydrogel composition wherein the embedded component comprises a microparticle.

In a two hundred and ninety-second embodiment, alone or in combination with any other embodiment herein, the present disclosure relates to a hydrogel composition wherein the microparticle comprises a biodegradable polymer.

In a two hundred and ninety-third embodiment, alone or in combination with any other embodiment herein, the present disclosure relates to a hydrogel composition wherein the biodegradable polymer is chosen from poly-glycolic acid (PGA), poly-lactic acid (PLA), poly-L-lactic acid (PLLA), polycaprolactone (PCL), poly-DL-lactic acid (PDLLA), poly(lactic-co-glycolic) acid (PLGA), poly(trimethylene carbonate) (PTMC), poly (ester amine)s (PEA), poly(para-dioxanone) (PPDO), poly-2-hydroxy butyrate (PHB), and co-polymers thereof.

In a two hundred and ninety-fourth embodiment, alone or in combination with any other embodiment herein, the present disclosure relates to a hydrogel composition wherein the microparticle further comprises a therapeutic agent.

In a two hundred and ninety-fifth embodiment, alone or in combination with any other embodiment herein, the present disclosure relates to a hydrogel composition wherein the microparticle further comprises a radioactive isotope or a compound including at least one radioactive element.

In a two hundred and ninety-sixth embodiment, alone or in combination with any other embodiment herein, the present disclosure relates to a hydrogel composition wherein the radioactive isotope comprises yttrium-90.

In a two hundred and ninety-seventh embodiment, alone or in combination with any other embodiment herein, the present disclosure relates to a hydrogel composition wherein the compound is chosen from yttrium phosphate (90YPO4), yttrium sulfate (90Y2 (SO4)3) or (89Y90Y(SO4)3), or yttrium carbonate (90Y2 (CO3)3) or (89Y90Y(CO3)3).

In a two hundred and ninety-eighth embodiment, alone or in combination with any other embodiment herein, the present disclosure relates to a hydrogel composition

In a two hundred and ninety-ninth embodiment, alone or in combination with any other embodiment herein, the present disclosure relates to a hydrogel composition

In a three hundredth embodiment, alone or in combination with any other embodiment herein, the present disclosure relates to a hydrogel composition wherein the microparticle further comprises a radiopaque marker.

In a three hundred and first embodiment, alone or in combination with any other embodiment herein, the present disclosure relates to a hydrogel composition wherein the radiopaque marker comprises methylated TIBA (TIBA-Me).

In a three hundred and second embodiment, alone or in combination with any other embodiment herein, the present disclosure relates to a hydrogel composition wherein the embedded component comprises a tissue marker.

In a three hundred and third embodiment, alone or in combination with any other embodiment herein, the present disclosure relates to a hydrogel composition

In a three hundred and fourth embodiment, alone or in combination with any other embodiment herein, the present disclosure relates to a hydrogel composition wherein the tissue marker is chosen from titanium particles, nitinol particles, PVA particles, or combinations thereof.

In a three hundred and fifth embodiment, alone or in combination with any other embodiment herein, the present disclosure relates to a hydrogel composition wherein the embedded component comprises a radiopaque compound.

In a three hundred and sixth embodiment, alone or in combination with any other embodiment herein, the present disclosure relates to a hydrogel composition wherein the radiopaque compound comprises methylated TIBA (TIBA-Me).

In a three hundred and seventh embodiment, alone or in combination with any other embodiment herein, the present disclosure relates to a method to prepare a hydrogel comprising: preparing a first solution of a monomeric unit, chosen from NHS-PEG-SCM-8, NHS-PEG-SCM-4, or NHS-PEG-SCM-2, reconstituted in a first aqueous solution; preparing a second solution of a complexing molecule reconstituted in a second aqueous solution, wherein the complexing molecule is polyethyleneimine (PEI); and contacting the first solution with the second solution as both are poured into a cavity space in a subject to form a polymerized complex therein.

In a three hundred and eighth embodiment, alone or in combination with any other embodiment herein, the present disclosure relates to a method to prepare a hydrogel wherein the cavity space is between the subject's rectum and prostate.

In a three hundred and ninth embodiment, alone or in combination with any other embodiment herein, the present disclosure relates to a method to prepare a hydrogel wherein the cavity space is located in the subject's breast tissue.

In a three hundred and tenth embodiment, alone or in combination with any other embodiment herein, the present disclosure relates to a method to prepare a hydrogel wherein the complexing molecule is rHA or PEI.

In a three hundred and eleventh embodiment, alone or in combination with any other embodiment herein, the present disclosure relates to a method to prepare a hydrogel wherein the monomeric unit comprises a terminal structure, a linker and a core and wherein the complexing molecule is chosen from recombinant albumin, polyethyleneimine (PEI), and poly-lysine.

In a three hundred and twelfth embodiment, alone or in combination with any other embodiment herein, the present disclosure relates to a method to prepare a hydrogel wherein the linker comprises polyethylene glycol (PEG).

In a three hundred and thirteenth embodiment, alone or in combination with any other embodiment herein, the present disclosure relates to a method to prepare a hydrogel wherein the linker provides a molecular weight (MW) of between 1 kDa and 100 kDa.

In a three hundred and fourteenth embodiment, alone or in combination with any other embodiment herein, the present disclosure relates to a method to prepare a hydrogel wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

In a three hundred and fifteenth embodiment, alone or in combination with any other embodiment herein, the present disclosure relates to a method to prepare a hydrogel wherein the core is chosen from pentaerythritol, hexaglycerol, tripentaerythritol, or glycerol.

In a three hundred and sixteenth embodiment, alone or in combination with any other embodiment herein, the present disclosure relates to a method to prepare a hydrogel wherein the monomeric unit has 2, 4, or 8 linker arms extending from the core.

In a three hundred and seventeenth embodiment, alone or in combination with any other embodiment herein, the present disclosure relates to a method to prepare a hydrogel wherein an ester of the monomeric unit complexes with an amine or imine of the complexing molecule.

In a three hundred and eighteenth embodiment, alone or in combination with any other embodiment herein, the present disclosure relates to a method to prepare a hydrogel wherein a molar ratio of ester of the monomeric unit to amine or imine of the complexing molecule is of from 0.05 to 3.

In a three hundred and nineteenth embodiment, alone or in combination with any other embodiment herein, the present disclosure relates to a method to prepare a hydrogel wherein the molar ratio is of 1.5 to 2.

In a three hundred and twentieth embodiment, alone or in combination with any other embodiment herein, the present disclosure relates to a method to prepare a hydrogel wherein the complexing molecule comprises from 2 to 60% weight/volume (w/v) of the hydrogel.

In a three hundred and twenty-first embodiment, alone or in combination with any other embodiment herein, the present disclosure relates to a method to prepare a hydrogel wherein the monomeric unit:complexing molecule mass ratio is provided at 48:1.3, 26:1.3, 25:1.3, 13:1.3, 13:54, 1:1, 13:13.5, 13:27, 21:1.3, 10:1.3, 39:1.3, 64:1.3, 40:0.8, 30:0.6, 40:0.8, 30:0.9, 17:0.6, 40:0.8, 20:0.4, 40:0.8, 40:1, 40:0.2, 40:0.5, 31:1.3, 20:1.2, 20:0.2, 10:0.5, 20:0.9, 33:1.2, 33:1.1, 33:1.4, 33:1.5, 33:1.3, 33:1.6, 33:1.7, 33:1.8, 33:1.9, 33:2, 36:1.4, 29:1.4, 23:1.4, 36:0.9, 20:0.8, 20:0.6, 13:0.4, 18:0.6, 25:0.5, 20:1.2, 25:1.6, 23:1.3, 22:1.6, 22:1.4, 25:0.7, 25:0.8, 23:0.7, 13:0.4, 18:0.6, 22:1.3, or 23:0.7.

In a three hundred and twenty-second embodiment, alone or in combination with any other embodiment herein, the present disclosure relates to a method to prepare a hydrogel wherein the hydrogel has a pH of between 7.5 and 11.0.

In a three hundred and twenty-third embodiment, alone or in combination with any other embodiment herein, the present disclosure relates to a method to prepare a hydrogel wherein the monomeric unit is NHS-PEG-SG-8, NHS-PEG-SG-4, or NHS-PEG-SG-2.

In a three hundred and twenty-fourth embodiment, alone or in combination with any other embodiment herein, the present disclosure relates to a method to prepare a hydrogel wherein the monomeric unit is NHS-PEG-SS-8, NHS-PEG-SS-4, or NHS-PEG-SS-2.

In a three hundred and twenty-fifth embodiment, alone or in combination with any other embodiment herein, the present disclosure relates to a method to prepare a hydrogel wherein the monomeric unit is NHS-PEG-SCM-8, NHS-PEG-SCM-4, or NHS-PEG-SCM-2.

In a three hundred and twenty-sixth embodiment, alone or in combination with any other embodiment herein, the present disclosure relates to a method to prepare a hydrogel wherein the complexing molecule is rHA.

In a three hundred and twenty-seventh embodiment, alone or in combination with any other embodiment herein, the present disclosure relates to a method to prepare a hydrogel wherein the complexing molecule is PEI.

In a three hundred and twenty-eighth embodiment, alone or in combination with any other embodiment herein, the present disclosure relates to a method to prepare a hydrogel further comprising a component to be embedded within the hydrogel within the first solution and/or the second solution.

In a three hundred and twenty-ninth embodiment, alone or in combination with any other embodiment herein, the present disclosure relates to use of a hydrogel composition to separate a targeted tissue from a non-targeted tissue in a subject, wherein said use comprises implanting the hydrogel in a cavity space between the targeted tissue and non-targeted tissue.

In a three hundred and thirtieth embodiment, alone or in combination with any other embodiment herein, the present disclosure relates to use of a hydrogel composition wherein the cavity space is located between the rectum and the prostate.

In a three hundred and thirty-first embodiment, alone or in combination with any other embodiment herein, the present disclosure relates to use of a hydrogel composition wherein

In a three hundred and thirty-second embodiment, alone or in combination with any other embodiment herein, the present disclosure relates to the cavity is located in the breast tissue.

In a three hundred and thirty-third embodiment, alone or in combination with any other embodiment herein, the present disclosure relates to use of a hydrogel composition wherein the hydrogel exerts a therapeutic effect on and/or enhances a treatment of the targeted tissue.

In a three hundred and thirty-fourth embodiment, alone or in combination with any other embodiment herein, the present disclosure relates to a kit comprising a first aqueous solution, a second aqueous solution, a dried monomeric unit chosen from NHS-PEG-SCM-8, NHS-PEG-SCM-4, or NHS-PEG-SCM-2, and a dried complexing molecule comprising PEI, wherein the first aqueous solution reconstitutes the monomeric unit and the second aqueous solution reconstitutes the complexing molecule.

In a three hundred and thirty-fifth embodiment, alone or in combination with any other embodiment herein, the present disclosure relates to a kit comprising a means to mix the first aqueous solution and the second aqueous solution as prior to application into a cavity space within a subject.

In a three hundred and thirty-sixth embodiment, alone or in combination with any other embodiment herein, the present disclosure relates to a kit wherein the means comprises a first syringe barrel and a second syringe barrel both operably connected to a single needle point.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1-32. (canceled)

33. A method to prepare a hydrogel composition comprising:
preparing a first solution of a monomeric unit reconstituted in a first aqueous solution;
preparing a second solution of a complexing molecule reconstituted in a second aqueous solution;
suspending a microparticle in the first solution and/or second solution; and
contacting the first solution with the second solution as both are poured into a cavity space in a subject to form a polymerized complex therein.

34. The method of claim 33, wherein the cavity space is between the subject's rectum and prostate.

35. The method of claim 33, wherein the cavity space is located in the subject's breast tissue.

36. The method of claim 33, wherein the complexing molecule is rHA or PEI.

37. The method of claim 33, wherein the monomeric unit comprises a terminal structure, a linker and a core and wherein the complexing molecule is chosen from recombinant albumin, polyethyleneimine (PEI), and poly-lysine.

38. The method of claim 37, wherein the linker comprises polyethylene glycol (PEG).

39. (canceled)

40. The method of claim 33, wherein the terminal structure is selected from N-hydroxysuccinimide (NHS) carboxymethyl ester (NHS-SCM), NHS succinate ester (NHS-SS), NHS glutarate (NHS-SG), or triiodobenzoic acid (TIBA).

41. The method of claim 33, wherein the core is chosen from pentaerythritol, hexaglycerol, tripentaerythritol, or glycerol.

42-53. (canceled)

54. The method of claim 33, wherein the microparticle comprises a biodegradable polymer.

55. The method of claim 54, wherein the biodegradable polymer is chosen from poly-glycolic acid (PGA), poly-lactic acid (PLA), poly-L-lactic acid (PLLA), polycaprolactone (PCL), poly-DL-lactic acid (PDLLA), poly(lactic-co-glycolic) acid (PLGA), poly(trimethylene carbonate) (PTMC), poly (ester amine)s (PEA), poly(para-dioxanone) (PPDO), poly-2-hydroxy butyrate (PHB), and co-polymers thereof.

56. The method of claim 33, wherein the microparticle further comprises a therapeutic agent.

57. The method of claim 56, wherein the therapeutic agent is a chemotherapeutic agent chosen from docetaxel, degarelix, abiraterone, apalutamide, bicalutamide, cabazitaxel, darolutamide, leuprolide, enzalutamide, flutamide, goserelin, lutetium Lu¹⁷⁷ vipivotide, tetraxetan, olaparib, mitoxantrone, nilutamide, relugolix, sipuleucel-T, radium 223, rucaparib camsylate, or combinations thereof.

58. The method of claim 56, wherein the therapeutic agent is a chemotherapeutic agent chosen from raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, or combinations thereof.

59. The method of claim 56, wherein the therapeutic agent comprises a radiosensitizing agent chosen from gemcitabine, fluorouracil or 5-fluorouracil, interferon-α, 13-cis-retinoic acid, doxorubicin, docetaxel, carboplatin, cisplatin, dactinomycin, methotrexate, bleomycin, hydroxyurea, cetuximab, nimotuzumab, AMG102, paraoxonase-2 C-reactive peptide, gold, GSH-modified gold, silver, bismuth, palladium, gadolinium, xinc, curcumin, misonidazole, tirapazamine, paclitaxel, resveratrol, mitomycin C, etanidazole, AQ4N, lidocaine, procaine, chloropromazine, fludarabine, motexafin, nicotinamide, and combinations thereof.

60. The method of claim 56, wherein the therapeutic agent comprises a radioprotective agent chosen from amifostine, palifermin, super oxide dismutase, tetracycline, genistein, captopril, Lisinopril, 3,3′-diindolylmethane, rapamycin, CBLB502, ON01210, Y-tocotrienol, 8-tocotrienol, R-spondin 1, transforming growth factor β3, mesenchymal stem cells, bone marrow stromal cells, myeloid progenitor cells, antioxidants, or combinations thereof.

61. The method of claim 33, wherein the microparticle further comprises a radioactive isotope or a compound including at least one radioactive element.

62. The method of claim 61, wherein the radioactive isotope comprises yttrium-90.

63. The method of claim 61, wherein the compound is chosen from yttrium phosphate (⁹⁰YPO₄), yttrium sulfate (⁹⁰Y₂ (SO₄)₃) or (⁸⁹Y₉₀Y(SO₄)₃), or yttrium carbonate (⁹⁰Y₂ (CO₃)₃) or (⁸⁹Y₉₀Y(CO₃)₃).

64-73. (canceled)
